(19) United States
(12) Patent Application Publication
(10) Pub. No.: US 2004/0175719 A1
(43) Pub. Date: Sep. 9, 2004

(54) Synthetic tag genes

(75) Inventor: Frederick C. Christians, Los Altos, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/619,739

(22) Filed: Jul. 14, 2003

Related U.S. Application Data
(60) Provisional application No. 60/395,530, filed on Jul. 12, 2002.

(51) Int. Cl. C12Q 1/68; C07H 21/04; C12P 19/34; C12N 9/22

(52) U.S. Cl. 435/6; 435/91.2; 435/69.1; 435/199; 435/320.1; 435/325; 536/23.2

(57) ABSTRACT

In one aspect of the invention, a method to construct a synthetic “gene” composed of linked synthetic Tag gene sequences is provided. In one embodiment, the genes, about 500 to 4000 base pairs long, are made by annealing and extending overlapping 60mer oligonucleotides followed by cloning into a plasmid vector. Both poly(A)-tailed sense (Tag) RNA and antisense (Tag Probe) RNA can be produced from the clones by in-vitro transcription. In another embodiment, the genes can be used as exogenous spikes for any sample. In another aspect of the invention, these synthetic gene spikes can serve as normalization controls in gene expression monitoring experiments and can also be used to assess system specificity, sensitivity, and dynamic range. These synthetic Tag genes are thus useful in assay development, in product development and validation, and for quality control.

This application claims the benefit of U.S. provisional application 60/395,530, filed Jul. 12, 2002, the disclosures of which are incorporated here by reference in their entirety for all purposes.

### FIELD OF INVENTION

This invention relates in general to methods for nucleic acid analysis, and, in particular to, synthetic Tag genes useful as assay controls, in assay development, product development and validation, and for quality control.

### BACKGROUND OF THE INVENTION

New technology has enabled the production of microarrays smaller than a thumbnail that contain hundreds of thousands or more of different molecular probes. These techniques are described in U.S. Pat. No. 5,143,854, PCT WO 92/10092, and PCT WO 90/15070. Microarrays have probes arranged in arrays, each probe ensemble assigned a specific location. Microarrays have been produced in which each location has a scale of, for example, ten microns. The microarrays can be used to determine whether target molecules interact with any of the probes on the microarrays. After exposing the array to target molecules under selected test conditions, scanning devices can examine each location in the array and determine whether a target molecule has interacted with the probe at that location.

Microarrays wherein the probes are oligonucleotides (“oligonucleotide arrays”) show particular promise. Arrays of nucleic acid probes can be used to extract sequence information from nucleic acid samples. The samples are exposed to the probes under conditions that allow hybridization. The arrays are then scanned to determine to which probes the sample molecules have hybridized. One can obtain sequence information by selective tiling of the probes with particular sequences on the arrays, and using algorithms to compare patterns of hybridization and non-hybridization. This method is useful for sequencing nucleic acids. It is also useful in gene expression monitoring, i.e., monitoring the expression of a multiplicity of preselected genes.

There is a need for exogenous nucleic acid controls (“spikes”) for microarray analysis. While genotyping applications will benefit from the use of spikes, the need is especially acute for gene expression monitoring, in which the goal is to determine the quantity of each transcript species in a sample. Variations in sample preparation, hybridization conditions, and array quality are just some of the factors that influence the values determined for the transcript levels of different samples. Constructing large databases of samples prepared differently and hybridized to different array types becomes especially challenging. The use of quality-assured control polynucleotides during sample preparation and during hybridization to microarrays greatly enhances the ability to normalize data and to compare experiments, as well as to monitor each step of the assay. Many other applications can also benefit from control spikes. One advantage comes from starting with defined quantities of spiked polynucleotides of known sequences.

### SUMMARY OF THE INVENTION

In one aspect of the invention, a method to construct a synthetic “gene” composed of linked synthetic Tag gene sequences is provided. In one embodiment, the genes, about 500 to 4000 base pairs long, are made by annealing and extending overlapping 60mer oligonucleotides followed by cloning into a plasmid vector. Both poly(A)-tailed sense (Tag) RNA and antisense (Tag Probe) RNA can be produced from the clones by in-vitro transcription. In another embodiment, the genes can be used as exogenous spikes for any sample. In another aspect of the invention, these synthetic gene spikes can serve as normalization controls in gene expression monitoring experiments and can also be used to assess system specificity, sensitivity, and dynamic range. These synthetic Tag genes are thus useful in assay development, in product development and validation, and for quality control.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1. Synthesizing genes from oligonucleotides. A) Each 60-mer oligonucleotide is designed to overlap by 20 bases two different oligonucleotides encoding the opposite strand. In this case the left-most antisense oligonucleotide circularizes the assembly by annealing to the 5′ end of the leftmost sense oligonucleotide and to the 3′ end of the rightmost sense oligonucleotide. B) Extension of the annealed oligonucleotides by DNA polymerase results in a spiral concatamer. C) Multiple rounds of extension, with replenishment of nucleotides and polymerase each round, can yield products over 50 kb in length (the largest marker band is 12 kb). Assembly of five different genes is shown here. D) PCR or restriction endonuclease digestion of a concatamer can yield a single monomer, which can then be cloned into a vector.

FIG. 2. Tag clone arrangement in a plasmid vector. Each Tag gene consists of linked GenFlex™ (Affymetrix, Inc., Santa Clara, Calif.) Tag sequences, arranged so that transcription from the T3 promoter makes poly(A)-tailed sense (Tag) RNA, and T7 transcription makes antisense (Tag probe) RNA.

FIG. 3. BigTag clone arrangement in a plasmid vector.

FIG. 4. Using TagI-Q plasmid a control for long-range PCR. The PstI-linearized plasmid is depicted in panel A. Three primer-binding sites and two PCR amplicons are indicated. Panel B gives the sequences of the primers that are used to produce the PCR products shown in panel C (the two PCRs were performed in triplicate). Plasmid TagI-Q and the primers can be used as quality-assured reagents to control for the long-range PCRs, fragmentation, labeling, and/or hybridization steps in genotyping assays.

FIG. 5. Site-directed mutagenesis added restriction endonculease recognition sites for XbaI (“X”) and for EcoRI (“E”) to pTagIQ to create plasmid pTagIQ.EX (panel A). Panel B is an agarose gel demonstrating the presence the expected products following XbaI/EcoRI double digests.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example hereinbelow. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, Biochemistry, (WH Freeman), Gait, “Oligonucleotide Synthesis: A Practical Approach” 1984, IRL Press, London, all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Applications Nos. PCT/US99/00730 (International Publication Number WO 99/36760) and PCT/US 01/04285, and in U.S. patent applications Ser. Nos. 09/501,099 and 09/122,216 which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping, and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefor are shown in U.S. Ser. No. 10/013,598, and U.S. Pat. Nos. 5,856,092, 6,300,063, 5,858,659, 6,284,460 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. For example, see the patents in the gene expression, profiling, genotyping and other use patents above, as well as U.S. Ser. No. 09/854,317, Wu and Wallace, Genomics 4, 560 (1989), Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), Guatelli et al., Proc. Nat. Acad. Sci. USA, 87, 1874 (1990), WO 88/10315, WO 90/06995, and U.S. Pat. No. 6,361,947.

The present invention also contemplates detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201 639; 6,218,803; and 6,225,625 and in PCT Application PCT/US99/06097 (published as WO99/47964), each of which also is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over the internet. See provisional application 60/349,546.

I. Synthetic Tag Genes

In accordance with one aspect of the present invention, synthetic genes are made using Affymetrix GenFlex™ (Affymetrix, Inc., Santa Clara, Calif.) Tag sequences. Tag sequences are 20mer probes which were selected from all possible 20mers to have similar hybridization characteristics and minimal homology to sequences in the public databases. See, e.g., U.S. Pat. No. 6,458,530 (incorporated here by reference). The list of the reverse complements corresponding to the Tag sequences (also sometimes called the Tag probes) used to construct the Tag genes is set forth below in Seq. Id. Nos. 1-2050

| | |
|---|---|
| Seq. Id | 3′ to 5′ sequence |

| | |
|---|---|
| +TA,1 | TAAACTAGCATTGAGCCCAC |
| 2 | AAATCAGCAAACGGGCTCCG |
| 3 | GAATTGATAATCGCAGCCAC |
| 4 | GATATAGGAATGGCGCATAC |
| 5 | CTCATCGGAAGGGCTCGTAA |
| 6 | ACAGATGGAAAGGCAGTTCT |
| 7 | TTTGGTAGCTGAGTGCCCTA |
| 8 | TAACTGGTTTGACGCCACGC |
| 9 | TAATTGAGCTGACGGCGCAC |
| 10 | TTGTTGCTACTCTGGCCCGA |
| 11 | TTCCGTGCATAGTATAGGGA |
| 12 | TTATGCGACTTATCTCGGGA |
| 13 | TGTATAGGATTATGTCCGCG |
| 14 | CTGCTAGGAATATGAGCTAC |
| 15 | CTTCTGTCAATATGGGTACG |
| 16 | TATTTCGAGATATGAGGCGC |
| 17 | TTGATCGTAGATTCGTGAGC |
| 18 | CGAGATTACAATTCACGAGC |
| 19 | TGGTGTCTAGCTTCCAGCCT |
| 20 | TCAGGTCACGGTTCATGCTA |
| 21 | TGGTTACTGGTATATGCCGC |
| 22 | CCGAGTGCAGAATAAACCCG |
| 23 | GCGGTCTCAATACAAACTCA |
| 24 | GAAGCTACCATACGCGAGCA |
| 25 | ACGGGATAACAACGCAGCCT |
| 26 | AGAAGATCAACAGCTCGTCC |
| 27 | ATAAGATCAAGACCTGTGCC |
| 28 | ATTAGATTAAGACCAGCGCC |
| 29 | ATATAATCAAGACTGGCGCG |
| 30 | AGCATATAACCACTGATCCG |
| 31 | ACACTATTAAAGCTGCTCCG |
| 32 | CAATGTATAAGACTCTCGCC |
| 33 | CACTAATTCAGACGAAGCCG |
| 34 | GACCCTATCAGACAGATGCA |
| 35 | CACGCATCAAGACAGTATCG |
| 36 | CAGCTCCTAAGACTTGGACA |
| 37 | GGTATCATAGGACATTCGCA |
| 38 | GGTTACATGGATATAGCACC |
| 39 | TGTGTTTCAGCTATGCAGGC |
| 40 | TAATTCGCTGCAACCAGATC |
| 41 | ATAATTCCAACATGGGAGCC |
| 42 | CATTGCTTAATATGGGAGCC |
| 43 | CAATGCTTAATACCGACACG |
| 44 | GATTGCTTAGACCCTGCACG |
| 45 | GATTCATTAGACCAGGCGCT |
| 46 | GATTCTACATGCCACTAGCA |
| 47 | CCTGCGAACTGGCCTGAATA |
| 48 | CGCAGCGGAAGGCTCAATAA |
| 49 | CCTACCGCAAGGCAGGATAA |
| 50 | CCTATGATAAGGCACGCACA |
| 51 | CGCTGTGCAAGGCTCGTATA |
| 52 | CGATTGTCAAGGCAGTGATA |
| 53 | CATTGCGAACTGCATCTAAC |
| 54 | GATAGTCCAATGCTACTGAC |
| 55 | GATTCGGTAATGCGCTGTAA |
| 56 | GACGTTTCAATGCAGCGTAA |
| 57 | GAGAGTGCAATGCCGACTAA |
| 58 | GAGATCCGAATGCGCGTACT |
| 59 | CGAGATCCAAGGCCCATGAT |
| 60 | AGCTTGCACAGTAACCATGA |
| 61 | AGAGTTGAACAGCATACCCT |
| 62 | TATCTGATCGGACGGCCAGT |
| 63 | TATTGACTACTGCGCCTCAG |
| 64 | TTGGACTATTGGGTATCGCC |
| 65 | TTGTCAGATTGGATGCGCTC |
| 66 | TATGCAGAATGGCGTGTATC |
| 67 | CATTGGATAAGCACTGATCG |
| 68 | CCCGGAATAAGGCCACGATA |
| 69 | CTCATAGAATGGACCAGATC |
| 70 | CATAGATTAAGCACTCAGCC |
| 71 | CATGATGTAAGCACGCTACC |
| 72 | CAGGAGCGAAGCAGATACTC |
| 73 | CAGAGCAGAAGCACTCACGT |
| 74 | TACATAGGCTTCAGCATCAC |
| 75 | TATTATACCTTGATCCGCGC |
| 76 | TAAACTGCTTGCATACGGCG |
| 77 | TATAAGCCTTGCAGCGGACC |
| 78 | TTTAAGCGGTGGATCTAGCT |
| 79 | TTAATAGCCTTGAGCAGCGA |
| 80 | ATAAATGCTTGGAACCCTCG |
| 81 | GAAAGTTCATGGAATCGAGC |
| 82 | GCAAGGATTTCGACTCAGAC |
| 83 | CAAAGAATAATCGCTCCTCG |
| 84 | TAAAGCACTTATGACTCGGC |
| 85 | TTATAGCATTCTGTAGGCGC |
| 86 | TCGCTGACATTTGATTAGCC |
| 87 | CCTTGAATAATATCTCGGCC |
| 88 | AGGTCCAGAAATTGCTGCAC |
| 89 | AGCTCAGGAAATTCTAGCGA |
| 90 | AGCTATGCAAATTAGAGGCC |
| 91 | GGTAGGCTAATTTATGGCAC |
| 92 | GTAATGCAATTCAATGCCGC |
| 93 | CAACTGGCAATCAATACGCT |
| 94 | CCAAGCGAATGCAACGTATC |
| 95 | GCATAGCGAATTGGAGATAC |
| 96 | GCATGTCGAATGGATGATAC |
| 97 | GCACGTTCAATGGCTCGACT |
| 98 | GCAGCGCAATCTGTCGAGTA |
| 99 | AGCAGTGCAAATCCTGATAC |
| 100 | AGCTTCGCAAATCTGGTACA |
| 101 | AGCCTGCGAAATCTACTGAA |
| 102 | GCAGATCGAATTATGGAGAC |
| 103 | GCAGAGTCAATTATCATGCC |
| 104 | CGTTAGGCAATACATTTCCC |
| 105 | ACTGGTGCAAAGTCTTCGAC |
| 106 | GGTATATGAATGTGTCGTCC |
| 107 | GATAGTGCAATCTAGGTGAC |
| 108 | GCAGTGCAATGGATGTACTA |
| 109 | GCTAGGCTAATGTCCGGCTA |
| 110 | GGTAGCCTAATGTGTGCTCA |
| 111 | GGACGTGCAATCTTGTGACC |
| 112 | GAGCGCCGAATCTAGTCGAA |
| 113 | GGGAGCGACCTCTAGCTTAT |
| 114 | GCGGGTCGAATCTCGCTTAA |
| 115 | CGCCGCGCAAGCTGTATTAA |
| 116 | CGGCTGCGAAGCTGTCTTAA |
| 117 | CATCCGCTAAGATCGGTTAA |
| 118 | CGTGCAGCATAATCCATCAG |
| 119 | TGAGAGCTGGATCGCATTCC |
| 120 | TAGGTGCTAGGATCTCAGCC |
| 121 | TAGGTATCAGGATTCAGGCC |
| 122 | TGCGCCAGTGAGTGGTATAT |
| 123 | CAGCAACGTGGATCAACTAT |
| 124 | CAGCGGCTAAGATCAATACC |
| 125 | GCAGCCTAATCTGGCCTAGT |
| 126 | GGGCCTGTACCTGCAATTCA |
| 127 | TAGGCCGGACCTGCTGTTAT |
| 128 | TAAGCCGCCACGGAGTGTTA |
| 129 | TAAGGCTCTTGAGACGTAGT |
| 130 | TAAGCCCGATCAGCATGGAC |
| 131 | TTGCCCGTAGTCAGCTTAGA |
| 132 | GAAGCACCGATCAGACACTG |
| 133 | CAGGCACCAAGTAGCACAGT |
| 134 | GGTGCGCCATGTACTCAGTT |
| 135 | TCAGGCTTATCGAGCGCGTT |
| 136 | GCAGGCAGATCGACCTAGTT |
| 137 | GGATAGGGACTCAGATATAC |
| 138 | GCATGGTTACCTACGCCAGA |
| 139 | GGAGGCTGACTCATACGCAA |
| 140 | GGAGCCTGACCTAGTCGATA |
| 141 | GCGGCCAATTCGGCGATAAT |
| 142 | GGTGCTCGACATTAGGCCAT |
| 143 | GATCCCACATAGCGGACAAT |
| 144 | GATCCAATCTGTCAGCACAT |
| 145 | GAGCCAATCTGACTACCAGT |
| 146 | TGCTGGATATGACTGTCGTA |
| 147 | TGCTCTGCACTGCTGACGTA |
| 148 | TCACCAGCCAGACTGTGTAG |
| 149 | AGGAGCAACCATCATGCACG |
| 150 | GGGCATACCTATCCCGAGAT |
| 151 | CGGGCGATACCACTCAGATT |
| 152 | AGCGGCAACCAGACATACGT |
| 153 | CACGCCATACCAAGGAGAGT |
| 154 | CAGTGCATACCAAGCGACGA |
| 155 | CAGGCAGTACACAATCTACG |
| 156 | TACGTCGCATCCATAGCTGA |
| 157 | GAGTGACACCTCAGCAGATA |
| 158 | CTACAGCACCTCAGGAGAGT |
| 159 | CTCACGACATCCAGGAGTAT |
| 160 | CCAGCACGACAGAGAGATGT |
| 161 | CGCACACACCTGAGAGAGAT |
| 162 | GCGCACGCACTCAGATGTAA |
| 163 | AGACGCTCAACCACGAGAGT |
| 164 | GACGCCACAGTCACTAGAGA |
| 165 | GGCGCACACTGTACTCAGAT |
| 166 | CGAAGCGCCAGTACCAGATA |
| 167 | GGGTCGCTACCTACTCTGAT |
| 168 | GAGACATGATCTACCAGTAC |
| 169 | GGACGCTTACTCAGCAGTCA |
| 170 | CGGGTGTTACAGAGCTATCA |
| 171 | CGCGGCTTACACAGACATTA |
| 172 | CGGAGCTTACACATTAGCAG |
| 173 | CTGAGCATACACTTCACGAT |
| 174 | CCGATCATAACTGTAGATGC |
| 175 | CCGCCGATAACTGCTTGAGA |
| 176 | GGCCATATACGAGATGTAGA |
| 177 | CGTCCCTTAACGGCTGGTAT |
| 178 | ATACCCAGAACGACTATGCG |
| 179 | ATCCCACGAACGATGAATCT |
| 180 | ATCCGCAGAACCGGCGATAA |
| 181 | CCTCGCCGAAGCGTGTTTAA |
| 182 | GCGCCGCACAGAGTCTTATA |
| 183 | CGCGCTGCACAGAGCATATA |
| 184 | CCGCTGACACAGGCAGATAT |
| 185 | GCGTATGACCAGGTGTATAT |
| 186 | CTGTATGAAGGTGCTGTACT |
| 187 | GTTTCGCACGAGGATGTATC |
| 188 | GTGCTCGCAGAGGATTTATC |
| 189 | TAGGCCAGAGTAGCGACTTA |
| 190 | CAGATCCTAAGAGCAGTTAC |
| 191 | TAGATGCTAGGAGCGATTCA |
| 192 | TAAGTCGGTGGAGCATATCA |
| 193 | TAAGCGCGTGGACTCCTAAA |
| 194 | TAAGTGGACTGAGCGCATAT |
| 195 | TATACGGCAGTGGATCAGAT |
| 196 | CTATACGCAATGCACTCAGA |
| 197 | CTATCGTCAAGTGATGGACC |
| 198 | TATAGACTAGGTGATCGAGC |
| 199 | TAGTACGAGTGGGCATCAAA |
| 200 | TAGACGTAGTGAGCATGACT |
| 201 | TGACGAGTTAGGATCTATGC |
| 202 | TTACGAGTGTAGCGTCCATG |
| 203 | TCGTCGTAGCATCTCGCAGT |
| 204 | TCGACGTAGGATCGCAGTAC |
| 205 | TCAGTATCATGGAGTACGAG |
| 206 | TGCACTAGATGGGATCGACT |
| 207 | TGCGATTACTGCCGTCACGT |
| 208 | TGGACTCTATGGCAGCCGTA |
| 209 | TGACAGCAGTTGCAGTCCGT |
| 210 | TACACAGGCTTGCAGCTCGA |
| 211 | TGCAGCGGAGTGCCTCATTA |
| 212 | GCGCAGGGAGATCCATATCA |
| 213 | CGGCAGCCAAGTCCAGTATA |
| 214 | CAGCGCCCAAGACGTGTATA |
| 215 | GTGCCTGCATAGCGATAGTC |
| 216 | TGCCTGCGAGAGCCTGTATT |
| 217 | TGGCATCGAGAGCCGTTCTA |
| 218 | GCAGGAGCAGAGCTTATATC |
| 219 | GCGGGATCACGACGTTTACA |
| 220 | GTGGCGATAGAGCATTCTCC |
| 221 | AACGCGAGAAACCATTTGCC |
| 222 | AGGCAGACAACTCAATCCGG |
| 223 | AGGAGAGCAACCTACACTCG |
| 224 | AGCCAACGAACCTACATGGG |
| 225 | CCGCAAGCACGTCGAATGAA |
| 226 | GCGCATGGACGACAAACGTA |
| 227 | GCCAGGAGACGTAGATATTA |
| 228 | GCGCATAGAGAGAGATCATC |
| 229 | TGGTATATCGGTAGATTCGC |
| 230 | GAGCTATAAGGTGGATTCAC |
| 231 | CGCGGATAACTTGATTCACC |
| 232 | GTCGGCTTACCTGATAGCGA |
| 233 | GGAGCTATACATGCCTATCC |
| 234 | GGTGCCGTACATGCTCGTAT |
| 235 | TCGGCTTGACGTGCTCGTAT |
| 236 | GGGCTGTGACTAGACTCTCA |
| 237 | GCGAATTTAGTAGACGCACA |
| 238 | GAATCTCGAATAGCGGTACA |
| 239 | GACAGTTGACATGACAGTAG |
| 240 | GACATTGACATCGCATACAC |
| 241 | GAGTTTAGAATCGTGAGCAC |
| 242 | CTATTCGCAAGTGTCGAGCC |
| 243 | GTTATGGACACTGCTCGACG |
| 244 | AGCGTTCTAAATGCGTCACA |
| 245 | CCGATATGAACTGTCACTAC |
| 246 | CGCGAATGAAGTCTACATAC |
| 247 | CCACTATGAAGCGATATACC |
| 248 | CACCAGTGAAGAGATACCGC |
| 249 | GCACTGTTACATGATACCTC |
| 250 | GCCAGTTACAGTCATGCCTA |
| 251 | GCGCAGCTAGATCCACTGAT |
| 252 | GCGTGCGGAGACCTCATTTA |
| 253 | GCTCACGAGGCACGCTTTAT |
| 254 | GCGCCAGTAGCACGCTTATT |
| 255 | GGCTCAGTAGCACTCATCAT |
| 256 | ACTTGCACAGCACAATACGT |
| 257 | CGCCATACAGCACGATATTA |
| 258 | CCGCAGACAGCACGAGTATT |
| 259 | CCAAGGAGACTACACGATCT |
| 260 | GCACAGGTAGCTCGACGTAT |
| 261 | GTCAAGATGCTACCGTTCAG |
| 262 | CGATATGAAGCTCAGTGAAC |
| 263 | CCTATGAAGCTATCGCAACA |
| 264 | CTTATCACAGCATCCGAGAG |
| 265 | CCCGTGCAACGATTTGACAA |
| 266 | CGGCGGTTAAGTTCTAATCA |
| 267 | GGTCGAGCATGATAGCTTAT |
| 268 | GTGGTAGCAGCATAGCTTAT |
| 269 | TAGCGTGGAGCATCCTCAGT |
| 270 | CAACGGTGAGCAACTATCAG |
| 271 | CTGGTTCGAGCAATCTATCA |
| 272 | TCGGGTCTAGGATGCTCTAC |
| 273 | TCGATGCACTGATGTCACTA |
| 274 | TCGTATATCCCATGCGATCT |
| 275 | TACGGTCCAGCATCAGCTTA |
| 276 | ATCAGTCCAACCTACAGATG |
| 277 | ATCAACTGAACCTCATACGG |
| 278 | TACTTCTGAGCAGGGAGCTA |
| 279 | TAGTTATGAGCAGGCGTCCA |
| 280 | CTTGTGACATCAGCCACGAT |
| 281 | CACGGAGCAAGAGCACATCT |
| 282 | CACGGGTGAAGAGCCATACA |
| 283 | CAGGAGTTAATAGCTCATCC |
| 284 | TAAGATTAGTTAGCAGCGCC |
| 285 | GAGTGATTAGCAGACGCCAC |
| 286 | CGATGATTACCAATGCCACG |
| 287 | GACTGATTAGCACATCCACA |
| 288 | GATTATGTAGCACTATGCCC |
| 289 | GCTATATTACGAGCTATGCC |
| 290 | GTTTATATCGAGGCAGGCCA |
| 291 | GTTACTATCCGATCAGAGCG |
| 292 | CGTCATGTACCATCAAGTCG |
| 293 | GTTATCTACGGATCATGCGA |
| 294 | CTGCCGTAAGTCTCATGCGA |
| 295 | CTAGCCGAATACTGCATACA |
| 296 | CTGCGTCGAGAATCGCGTTA |
| 297 | CATACACGACAATAGCTTCG |
| 298 | GATACCGACTCATACATTGC |
| 299 | GATACCGCACGATCAGCAGA |
| 300 | GTATATGCAGACTACTGGAG |
| 301 | TATAGTCGATTATCCCAGCC |
| 302 | CATAGTACAATATCCCGACG |
| 303 | CTTGACAGCTACTACCAGTG |
| 304 | CTGAGACAGCTATCGACACA |
| 305 | CTGAGTAAGTCTTCCACACG |
| 306 | TCGGATATACTATGCGTCAG |
| 307 | CGTAGGATAGAATGCACAGT |
| 308 | CATGATACACACTCACGAGG |
| 309 | CGGAATCACGACTACATACG |
| 310 | GGGTATCACGAGTCACCTCA |
| 311 | GAGAGAATCGTATCACAGCC |
| 312 | GAGTATGTAATCTACCTGCC |
| 313 | GAGTAATCATAGTAGCAGCC |
| 314 | GACTATATCCAGCACCGAGG |
| 315 | GACATATAGCTCCACTCAGA |
| 316 | TAGACCTAGTTGCAGCGCGA |
| 317 | TACTACACGTTTCACGGCAG |
| 318 | GTACATATCTGTCACGCGCA |
| 319 | TAGTATATCCTACGCCGCTA |
| 320 | GAGTATATCGCAATGCCAGC |
| 321 | GAGTTGTCACATAGGCCACC |
| 322 | GACGCATGACATATTCCTAC |
| 323 | GAGACACTTGACAGTAGCCA |
| 324 | GGCTAGTTACTCAGATCACA |
| 325 | CGCAATAAGTCTAGCTCACT |
| 326 | CATGTACTAAGCAGTCACAC |
| 327 | CTAGTTAATGTCAATCCGGC |
| 328 | GACTGTGTAATCATTGCAGC |
| 329 | CGTTCGTGAATCAGCACAGC |
| 330 | ATTCGGTCACACAGCACAGA |
| 331 | ATCTGCTGACACACACTAAG |
| 332 | AGCTCGCTAAATATGTAGGC |
| 333 | ACTGTCGCAAATATCACACG |
| 334 | ACTGTCTGACCAACCAATAG |
| 335 | GTTACTAGCTGGACCTCAGA |
| 336 | TTATAGACTGGTGCGGAACA |
| 337 | TTAGCATACTGTGCGCGAAC |
| 338 | TGTGCTGACTTAGGTGGAAT |
| 339 | TCTCGGGACGTTGCGCTATA |
| 340 | TGTCCGCGACGTTGGCTATA |
| 341 | TGTTCGTGACTGTGCGCTAC |
| 342 | TGTCAGGTACTGGTCGCTAC |
| 343 | TTCATGTACTGTGGCTACCG |
| 344 | TTTACTAGAGTGGCGCATGA |
| 345 | TTAGATAGATGTTCGGCCAG |
| 346 | CTCAATAGATTATAGGCGCG |
| 347 | TCGAATCGCTGTTACGGAAA |
| 348 | TCAGACTAGGGTAGCGCATA |
| 349 | TCAGCAGTATGTAGGCAGTA |
| 350 | TAAGCCGGGTCACGCTATTT |
| 351 | TATGACCGATGTGCAGGTAT |
| 352 | TTAGCACGCTCGGCGATGTT |
| 353 | TTCACACGGTCTGCGAGCTT |
| 354 | CTTCAGACAGGAGGAGATAT |
| 355 | TCCAGCCGACGTGCGATTTA |
| 356 | TCCAGCGTACCTGCTTGTAG |
| 357 | CTCCAGTCAAGTGCTTCGAG |
| 358 | CTCCAGCGAAGTGATGAGAA |
| 359 | TGTCAGCGGATCGCCATATA |
| 360 | TCCATGCGAGGATCAGGTAT |
| 361 | TGCAAGCAGTTCTCAGCGTA |
| 362 | TGTAGGACCTGTGCTCACTG |
| 363 | TTTATCGCAGTGCTCAGGCT |
| 364 | TATGTCAGCAGGCCCAGCTT |
| 365 | TTCTCGTAGCTGCGCCTAGT |
| 366 | TATTCGAGCTAGGGACGCAT |
| 367 | TATTTATACTGCGAGCGAGG |
| 368 | GACCTTACACTGGCACGAGA |
| 369 | TACTGATAGCATGGGACGTT |
| 370 | TCGGATAGCAGTGCGCTCTA |
| 371 | GCTGATGCACGAGGCCATTA |
| 372 | GCTGGATCACGAGGCTCATA |
| 373 | CGCTTTGTACCAGGCCATAG |
| 374 | CGTGATTGACCAGACCCAGT |
| 375 | TACGCTGGATCAGACGGTCA |
| 376 | ATCCTGAACGCAGAGACACG |
| 377 | ATCGTTGCACCAGAACTACA |
| 378 | CTCTCAGGACCAGCATGATA |
| 379 | TCTGAGCGATCTGCCAGTCA |
| 380 | GGTGAGACCTATGTATATCG |
| 381 | TTAGAGTCTTAGGCATGTCG |
| 382 | TTATAGCCGTAGGCAGGTAC |
| 383 | CTCTAAGTATTGGACACGCA |
| 384 | GCTAGGATATAGGACACTGA |
| 385 | GCTATCGAATGTGCAGTACG |
| 386 | TCTATCCACTGCGGACGAGT |
| 387 | TCATACTCATGTGCAGCTCT |
| 388 | TCATCGAGATCGGCCACTGT |
| 389 | CTTATGATACCAGTCAGCAC |
| 390 | TATTGGTACGGAGTTAGCCC |
| 391 | GTAGATGACCCAGTTCCAGC |
| 392 | GGCTGTTACCGAGTCTCAGA |
| 393 | TGCTAGTTAGGAGTATCGCA |
| 394 | GGCTTACTAGCAGTCACGCA |
| 395 | CAGCATATAAGAGTCGTACC |
| 396 | GGCATCATAGACGCTACGCT |
| 397 | GAGTCAGCAATCGCAGCTAA |
| 398 | GATCAGTAATGCGGAGCAAC |
| 399 | TATCATAGATGCGGACGGAT |
| 400 | CAGTCCACAAGCGCGAGTAA |
| 401 | CGTAGCCCAAGTGCCGATAT |
| 402 | GACGCACCACAGGCTAGTAT |
| 403 | CTAGCATACCAGGCGAGAGT |
| 404 | AGTGCATCACAAGAGACTCG |
| 405 | GCCATAGACGAGGCAGTATC |
| 406 | GGAATACGCTGAGATATACG |
| 407 | GTTAATCGCTCAGCAGCATT |
| 408 | CACAAGCGACCAGAAGCGTT |
| 409 | TCTTATCGACCAGGGCGGTT |
| 410 | GACACTATCCCAGACGGAGT |
| 411 | TTACTAGGTTCAGCGCGATC |
| 412 | TTCAGATCCTCAGCGTAGTC |
| 413 | TCTCAGATATTCGTAGCAGC |
| 414 | TGTCTATTAGTAGCTGCGAG |
| 415 | TAGATACTCTGAGCTAGGAG |
| 416 | TGTCTCCAGATCGTGCGAGT |
| 417 | TTCGGTCTAGCTGGTAGCAT |
| 418 | ATCTGGCGAACAGGTGCATA |
| 419 | AATGCGCGAAACGGCGATAC |
| 420 | TTTGTCGCAGTAGTCGCATC |
| 421 | TGTTGTGCAGTCTCCAGGCA |
| 422 | CATTGTGAACTCTACGTCAG |
| 423 | CGGATGTCAAGCTCTCACAG |
| 424 | CTGCGGCAATACTCTCAGGT |
| 425 | ATGCGGAGAACCTCTGACAA |
| 426 | GCGCGTGAATCCTGTGACTA |
| 427 | GCGCTCTGAATCTGTGAGAA |
| 428 | GCGCTATGAATGTCAGCTAA |
| 429 | GCCGAGGTAATGTGATATAC |
| 430 | GCCGCGTGAATATGAAGATA |
| 431 | GCGGCGAGAATCTTCCGATA |
| 432 | GATGGTAGAATCTCTCTCAC |
| 433 | GCTGCGGGAGACTATCATCT |
| 434 | GCTGGATTACGATGCCATAG |
| 435 | GTTGATTCACGATGGCAGAT |
| 436 | CTTCACGCAAGTTGTCCAGA |
| 437 | CTTACGCCAAGTTGTCAGAA |
| 438 | CTTGCGTCAATAGTCTGAGA |
| 439 | CCTGTGCGAACTGTCTTACA |
| 440 | CTCAGTCCAAGTGGCTCAGA |
| 441 | CCATAGCGAAGCGCACAGTA |
| 442 | CCAGCACTAAGCGCAGATAG |
| 443 | CTCCGCCTAAGTGGCAGTAA |
| 444 | TGCGCCTGACGTTCGGATTA |
| 445 | TGTCCAGTAGCTTGAGAGTC |
| 446 | GCTCACAGAGTTTGATAGAC |
| 447 | GCTACAGGAGTGGATATTAC |
| 448 | GTGACAGTGGCAGATATAAC |
| 449 | TCGCACTGAGCTGTAATCGA |
| 450 | TCTTATGAGATGTAGCTCGC |
| 451 | TCCATCTAGCTGTAGCCGAA |
| 452 | GTCATAGCAGCTTAGACCTA |
| 453 | TTATGCTGACTGTGCTCGAC |
| 454 | TTAGTGCAGTATTAGTCGCG |
| 455 | TGTCTGACCTTGTAGCCGAC |
| 456 | TGTTGACACTTGCGTACCGG |
| 457 | TCTTAGCATGTGCGACGACG |
| 458 | GCTAAGCTCTTGCACTGACG |
| 459 | CATAAGACTTTCCAATCGCG |
| 460 | CTGAAGCAGTTTCCACGAAG |
| 461 | CTGAACCCGTTGCAGAGAGA |
| 462 | CGGAACCGATGGCACAATAT |
| 463 | GGTGACCGATGGCTACTCAT |
| 464 | ATGGCGCGAACCCTGTACTA |
| 465 | CATCGCGGAAGCCACGTATA |
| 466 | GACGGCAGAATGCAGTATAT |
| 467 | CGCGGAAGAAAGCATATTTG |
| 468 | CTCAAGGGCACGCAATCTAG |
| 469 | TCACAGGAGGCTCGACTCTA |
| 470 | CGACAAGGCATTCACACTAG |
| 471 | ATAAAGGTCATGCCAACCGC |
| 472 | TATAATGCGTTTCACGTCCC |
| 473 | TCTAATGCCTGACACGAAAC |
| 474 | TGAATGCCGTGACTCGTAAA |
| 475 | GTGGAGGCACTGCATCATAA |
| 476 | GTGGTGTGACCTCGCCATTA |
| 477 | GGAGATGCACTACGGACTAT |
| 478 | GAGGATCGAATACTGTCGTA |
| 479 | CGGAGAGCAAGTCATACGAC |
| 480 | GCAGGAGACGGACTATACTA |
| 481 | GAGCGTGTAATCCGATCTAA |
| 482 | CGATACGGAAGGCGCACTAA |
| 483 | CGATAGGTAAGGCGACTCAA |
| 484 | GATGTGGCACGACGATCATA |
| 485 | TGAGTAGGCAGTCCGATCTA |
| 486 | TGATAGGCAGTGAGTTCATC |
| 487 | TTATGGCGAGAGTTGTCATC |
| 488 | GTTTAGGCACGATGCTGTAT |
| 489 | GCGTTAGGACCATAGTCTAC |
| 490 | CCGATGCGACAATACGTTAG |
| 491 | TCTAGCGTCCCATAGCGTAG |
| 492 | CTGTCTGGACCATAGCAGCA |
| 493 | CTGCTTGCACGATGAGCGAA |
| 494 | TAGCCCGGACGATGTAGTCA |
| 495 | CCGCTACAAGCATTGGGAAT |
| 496 | CGGCTAGAAGAATGAATGCT |
| 497 | CCGATGATAAGCTAGTATGC |
| 498 | GCGGATAGACCATTATTGAC |
| 499 | GCCACTAGACCATCGGTGAT |
| 500 | GCACGCGGACCATCGTTTAT |
| 501 | GCCGCTCGACCATAGTGATA |
| 502 | GCCGAGTCACCATGCTGTAT |
| 503 | CACGGGTCACCAAGCGTATT |
| 504 | GACGGCGACCCAGGTTATAT |
| 505 | TGTGCGTCAGCAGTTAGTAT |
| 506 | GCTCGGCTACCAGTCGTTAT |
| 507 | CGCTGGACACCACTGTGATA |
| 508 | CGGTGGAGACCAGATTATAT |
| 509 | CGCGGGACACCAGCATATTA |
| 510 | GCTCGCGCATTAGCATATAA |
| 511 | GCTGACATCCACGCATTGAG |
| 512 | CGCTGATCCACCGAGATTAG |
| 513 | ACGCAACCAACAGCGAGTGT |
| 514 | CACAGACCACAAGCTATGGG |
| 515 | CCTAGCCCAAGGCATTAGAA |
| 516 | CCGTAGCTCCAAGGCATGTA |
| 517 | CAGTGCGCCAGAGCAAGTAA |
| 518 | GAGCCACCACGAGTCATGTA |
| 519 | GGTCACCACTCAGCGATGTA |
| 520 | GTGTGCCACTAGGCCGATTT |
| 521 | GGAGACCCGTAGGCATAATT |
| 522 | CGCTGTAAGGATGCTGAATA |
| 523 | GTCGTGCAGGATGCCATATT |
| 524 | GTTCCGCACGATGCCAGATT |
| 525 | GCTGCGACCATCGTCAGATA |
| 526 | GTCTAGCGATCATGCTCAAT |
| 527 | CTCTACGAATCATGCGGAAG |
| 528 | CTTAGATACTACGAGCACGA |
| 529 | GTGACGCTACGTGAGCCTAA |
| 530 | TACCGTGTACGTGAGCGCAT |
| 531 | TACTGCGACGTAGCGAGTCA |
| 532 | TACTAGGTACTCGCGGCACT |
| 533 | TACTGCGTACTCGGAGCATA |
| 534 | GCTCACGTACTCGACAGAAA |
| 535 | GTGTACTATGTAGCGAGATC |
| 536 | TAGTAGTACGCTGTCAGAGC |
| 537 | TGTCGTCGAGTCGTAGATAC |
| 538 | GTAGTACACGGAGTGATCCT |
| 539 | GTAGTACGAGCTGAGACTCT |
| 540 | GTGACTAGCTCGTAATTCTG |
| 541 | GAGACACGGTACTAGAGACT |
| 542 | CAACAGCGTCACAGACATGG |
| 543 | CTATGAGACCACCTCGATAT |
| 544 | ATTCGGCGACAACGCATTTA |
| 545 | GTTGCCGTACTAGGGATACT |
| 546 | GGCGCAGTACGATTGACTAT |
| 547 | GTGCGACGAGCTTGTCACTA |
| 548 | TGCGTGTGACTATTGATACG |
| 549 | CGTCTGCGAACTTTGCTACG |
| 550 | CTGTAGCGAAGTTCTCATAC |
| 551 | TCGGCGTTACGTGCTGACTA |
| 552 | TGAGCTATACTCGTCGTCAG |
| 553 | CCGATACTAAGCGTTACGAA |
| 554 | CGTCATACATAGGACTAGCA |
| 555 | CGCACGCTACAGACTATTAT |
| 556 | GCGAGCGTACTATACATAAC |
| 557 | GCGAGTCTACGACCTCTATA |
| 558 | CGGTACGCACGACAGTCATA |
| 559 | CGGTACATACGACTATACAG |
| 560 | CGCTAGATACACCACTGATA |
| 561 | CTCTAGGTACACTACTGCAT |
| 562 | CGTCAGAGACACTGGAATAG |
| 563 | CTGCGCGTACACTCGGATAT |
| 564 | CTGTCGCTACACTCGTGAGA |
| 565 | GTAGACGCCTAGTCAGATAG |
| 566 | GAGCGACTACGAGCCACTAT |
| 567 | GTGCGACTACGTGCATCACT |
| 568 | CGTAGGACACGAGCGTATAT |
| 569 | GGCGACGACGTGACTATACT |
| 570 | CGGTCACGACGACGAGATAT |
| 571 | GCGTCACACGAGCCGATATT |
| 572 | GTCGCTCACGATGCGGATTT |
| 573 | GACCGACAGATCGTGACATC |
| 574 | GACCACGTACATGAGCTGAC |
| 575 | GGCGACGTAGATGATATTCT |
| 576 | GAGACTGTAATCGCATATCC |
| 577 | GACTATGTAATCGAGCCTAC |
| 578 | GATAGTCGAATCGCGGATAA |
| 579 | TATACGGACTGCGCCCTAGA |
| 580 | TAGTCTAGCTGAGCCATCGA |
| 581 | GTATATGACCTAGTGCCACG |
| 582 | GTGTTGTACGATGTGCTCCA |
| 583 | GAGTCTGACATAGGGCACCT |
| 584 | GAGTTGCACGTAGACGATAC |
| 585 | GACTCGCGCATAGACACATG |
| 586 | GACAGGCTACGAGACTAGAT |
| 587 | GTGACGGCACTAGCAATATA |
| 588 | CTGCTCTGACACGCGAGTAT |
| 589 | CGGCTGTGACACGAGCTATT |
| 590 | CTGGTGCGACACGCCTATAT |
| 591 | GTCAGTGGACTAGCCCTACA |
| 592 | ATCGAGTCAACCGGCCTAGA |
| 593 | TCGATAGCCTACGTGCCGTT |
| 594 | GGAGACCTCTACGCACTGTT |
| 595 | GCGTGACAGCTCGCACTATA |
| 596 | GCGTAGCTCAGCGACATTAA |
| 597 | GCTATACGCACCGTCATGTA |
| 598 | CGCATACACTCAGCAGAGAT |
| 599 | CTACTTACAGCAGCGACGAG |
| 600 | ATCTCGACACAAGCTAATCG |
| 601 | CATCGGATACACGCATACAG |
| 602 | ACATACAACACCGCTTAGGG |
| 603 | TACTGAGTCCACGCTCGGTA |
| 604 | GATACAGCCTACGACCGGAT |
| 605 | GATACATTACTCGACACGCG |
| 606 | CGCTACAGAGATGCACAGAG |
| 607 | CCGACTGTAACTGCGATGAA |
| 608 | GGTGTTATACGTGCATAGCC |
| 609 | CTCGTATTAAGTGCGCTACC |
| 610 | TATAGTATCGAGGAGCGACC |
| 611 | GTATAGTACGTGATAGGCTC |
| 612 | GTACGATACGTGACTAGAGC |
| 613 | GTAGGTCGAGCTGCATACTC |
| 614 | TTACAGTAGTCTGCATCCCT |
| 615 | CTAGTCAAGTCTGCATACAG |
| 616 | CTGTCTAATACGGCCACATA |
| 617 | CTCGCAATACGTGTACCGTG |
| 618 | TCCGATCTACGTGACGGTGA |
| 619 | TCTCGCCGACGTGGTCTTAA |
| 620 | TCTGTCCACGTCGCGGTTAT |
| 621 | TCGTCCTGACTCGCTGGTAA |
| 622 | GTCCCTAGACTCGCAGTGAT |
| 623 | GCGACAGTAGCTGCAATGAT |
| 624 | GACGTAATATCGCCACATCA |
| 625 | GACGAGGTACAGCGCATACA |
| 626 | GCAGGTCTACGACGCATGAT |
| 627 | GCAGAGTACGGACGCATATC |
| 628 | GAGTAGATACAGGTCACGAT |
| 629 | GAGCGATCACACGTCCGATT |
| 630 | GGTCGCATAGACGTATCAGT |
| 631 | GGTGTCTCACGAGTATCGAC |
| 632 | GTAGGCTAGACGGTCCACTA |
| 633 | GACGGACACTGAGCACATAG |
| 634 | GACACCTATGTAGCAATGAC |
| 635 | CACAGTACAATAGCACCTGG |
| 636 | CACCAGAACGTAGGCACAGT |
| 637 | CACTACTCAAGAGCCAGTTA |
| 638 | CGCCGACGAATAGCCAGATA |
| 639 | GCCGCACTACTAGCGATGAA |
| 640 | GACCAGTTACGAGCAGCGAA |
| 641 | GATCACGTAGGAGCACCGTA |
| 642 | GTACGCAGAGGAGTCATCCA |
| 643 | GTCGCTGACTAGGATCACGT |
| 644 | TACGCAGACTCGGACTCGAT |
| 645 | GTCGCTATATCGGACCTAAC |
| 646 | ACTCGCATAAACGACAGTCT |
| 647 | TGGAGTCGAGTAGTACATAC |
| 648 | TACGACATGGTAGGACGCTA |
| 649 | TGACTTCTACGTGGCGATAT |
| 650 | TACGCTCCGAGAGGCGATTT |
| 651 | CACCTTCGACGAGCAAGAGT |
| 652 | TACGCTCGCTCAGCTTAGGT |
| 653 | TACGGCATCGACGCTATTGC |
| 654 | TACGGCGACTGAGATGCCAT |
| 655 | TACGTGCTAGGAGATGTAAC |
| 656 | TATCGTCTATCAGATTGCCC |
| 657 | TATCGTATCCACGTTCCGAG |
| 658 | GATCGTACATCAGTGTCCAC |
| 659 | GAGTCTATATCAGTAGCGAC |
| 660 | GTTAGTCGATCAGTAGAGCA |
| 661 | GTCCTACGATGAGTGACGCA |
| 662 | CGTCTTCTAAGCGTGCTGAA |
| 663 | GTCTCCTACCGTGAGCAGTA |
| 664 | ATCTCACTACAAGAGCCTAG |
| 665 | CTGTGACGACCAGACGCTTA |
| 666 | CTGAGCGTAAGTGATTGTAC |
| 667 | CTCGTAGCAATAGATTTCCC |
| 668 | CTACGTGCAATAGCAGCTCA |
| 669 | CCGGCAGTACAGATAAGTCA |
| 670 | CGCCGGATACAGAGTAATCG |
| 671 | CTCAGCATACATAGTACAGC |
| 672 | CCGAGCTTACAACGTGTGCA |
| 673 | GACGCATTACCACTGGCGAT |
| 674 | CAGGGTGTACCACGAAGCAT |
| 675 | CGGTGTTTACAGCAATCCAT |
| 676 | CTGGCTGCAATAGCGCGATA |
| 677 | TGGGCTACAGTTGCGCTCAT |
| 678 | TCTGGCATAGCAGGTGTCAC |
| 679 | GGGATTCTACCAGTTCGCAC |
| 680 | GAGGATGCAATCGTAGTCAA |
| 681 | AGGGATAACCATGCACACCG |
| 682 | CATGAAGACTTTGCACTACC |
| 683 | CGCCGACCAATGGGCATATA |
| 684 | CCCGAGCCAACTGGAGATAA |
| 685 | CCCGCAGCAACTGGGATTAA |
| 686 | GCCATAGGAGCAGCGATTTA |
| 687 | CCGCTTGCAGCAGACGATAT |
| 688 | CCGTTTGCAGACAGCCAGTA |
| 689 | CCGTTTACAATGAGCACACA |
| 690 | CGTTCTTTAATGAGCGACAG |
| 691 | CGAGCCTTAATGACGCACAA |
| 692 | GGCAGCATACTCACGATCAT |
| 693 | CTGCGAGCAATCAGCCGATA |
| 694 | CCGCAGCAAGCTATCGAGAA |
| 695 | CGGCGTTCAAGCAAACCGAA |
| 696 | CAGTTTACAAGCATATCCCG |
| 697 | CATTGACGAAGCATAGTTCC |
| 698 | CATAGTGCAAGCAGCGACAC |
| 699 | ATCTGTGCAACCATAGTACC |
| 700 | ACTTGAAATGAGAAGCCCGT |
| 701 | CAGGAGAAGCGAATAGCCTC |
| 702 | CCAGAGAGAGCAATATCCGC |
| 703 | CAAGGAATATACAGGCCCGC |
| 704 | CAGAACTGAATTACAGCGCC |
| 705 | CATCAGACAATTACAGCTCG |
| 706 | CACCCGATAAGAGCATACGG |
| 707 | CACTCCAGAAGCACGATAGG |
| 708 | CAGCACCGAAGCAGAAGTCT |
| 709 | CAGATCAGAAGCAGGACGCT |
| 710 | CAGACCATAAGCACAGGCGT |
| 711 | ACAACACAAATGGCGCGGCT |
| 712 | ACGCAGATAAATCACCTCGG |
| 713 | CAAGACAGAATACTCTCCGG |
| 714 | CACAATACAATAGGCTCGCG |
| 715 | CAATAAGACATAGGCCGCCG |
| 716 | CACAACGGATTAGAAGCGCG |
| 717 | GACATGATATGAGAATGCGC |
| 718 | AGCAAACTAAGAGCCGGGTC |
| 719 | AACAATACAACCGTCGGCGG |
| 720 | AAATAACTAACCGCCTGCGT |
| 721 | CAAACACGAAGAGCCTGTCG |
| 722 | CACTAATCAAGCGACAGGCG |
| 723 | CATATACCAAGCTATCAGCG |
| 724 | CACATTCAAGACGATCACGT |
| 725 | CACCTATGAAGAGACTCACG |
| 726 | AACTATATCAAAGCCCTGGC |
| 727 | ACAATACCAAATGCGCCGGG |
| 728 | AGAAACGCAAATGCCTCTCG |
| 729 | CGAAAGCATAATAGCGGTGC |
| 730 | GGCAGAATCTCGTGTACTAG |
| 731 | GGTACATTATGCTAGAGAGC |
| 732 | GATACATGATGATAGCAGCG |
| 733 | AGAACAGGAACATCGCTGCC |
| 734 | AGATAAGCAACATCCTGTCC |
| 735 | CATAAGCTAAGATCCTGGAC |
| 736 | ATTTAGCGAAGAAGCATGGC |
| 737 | ATAGCTCAATCAACGATGCG |
| 738 | TATATCGCATCCACTCTGGG |
| 739 | CATCTCCGAAGCACATTGAG |
| 740 | CATTCGTCAAGCACTTCAGA |
| 741 | CATTATCGAAGCACGGTACA |
| 742 | GATTCGGACAGCACGGCATA |
| 743 | GCTCCGGCAGTCACGATTAA |
| 744 | GACTGTCGAGCACCCATTGA |
| 745 | GATCGTCGAGCACGCCTAAT |
| 746 | GAGGTCAGACGACGCCTATA |
| 747 | GCGCGTATAGCTCTCCATAG |
| 748 | TAGCGAGTAGCACTTCGATA |
| 749 | CTAAGTGTAGCACCACATCA |
| 750 | GTAGATCGAGCAGCCAGTCT |
| 751 | GACATAGACCATACCACGTT |
| 752 | CGTCTTCGAGCAAGTGCAGT |
| 753 | CTCTCCGGCAGCGATATGTA |
| 754 | CCCTCAGCACGAGATATAAG |
| 755 | CCCTTGCGAAGCATTGCGAA |
| 756 | CTCCAGGCAATGAGAGCACA |
| 757 | CCCAGATCAAGCGATGCAGA |
| 758 | CTGAATCCAATGTACGTGAC |
| 759 | CGGCATTCAAGGTAGCGACA |
| 760 | GCCCGATTAAGGTGTGTCAA |
| 761 | GCCCGATCAATGGCTGCATA |
| 762 | CGCCATCCAAGGGCTGTATA |
| 763 | CGGATGCCAAGGGCTTCATA |
| 764 | GGTTGCGCCAGGTCATCTTA |
| 765 | GGTCCGGCATGGATCACTAA |
| 766 | GGCTGGCACATGATCGTATA |
| 767 | TGGTTGCACTTGGATCGAAA |
| 768 | TGATTGCCACTGCTCATACG |
| 769 | TGTTGATCCATGTCCATAGC |
| 770 | TTAAGGCACTTGATCTCAGC |
| 771 | GTAATGCCCTGGACCGCAAT |
| 772 | GTTAAGCCTTCCACGGCAAT |
| 773 | GTTGCGCCATTGAGCCAGAT |
| 774 | GTTGCCCACCTGAGACGTTA |
| 775 | AATGCGCCACAAAGCGAGTG |
| 776 | CACCGGCCAAGAAGTACAGT |
| 777 | CATCCGCCAAGCAGAGTGAA |
| 778 | CGTTGCCAATGCACGAGCTA |
| 779 | GATGGCTGAATGACGTTTAC |
| 780 | GATTGCCTAATGAGTCTGAC |
| 781 | AATCAGCCAAAGATGTGGGC |
| 782 | AATCATGCACAAAGTTCGCC |
| 783 | ATTTAGGCAAGAAGCGCACC |
| 784 | AATTGGCTAAAGAGCGCACC |
| 785 | ACATTGGCAAAGCGAACTCC |
| 786 | AATGGGAGAAAGCCGACTCT |
| 787 | TGTGCTGGAGCTTCAGTCAC |
| 788 | GTTGTGCAGGATTATCGACA |
| 789 | GCTTGCAGACGAGTCATCAC |
| 790 | GGATGGATACTAGCGACTCC |
| 791 | GCTATGGCACAGGCATCTAC |
| 792 | GGACTGGCACATCCCGTATA |
| 793 | GGATCGGACCATTCTCACTA |
| 794 | GGATGGCGACATGCTCACTA |
| 795 | GAGCTGGCAATCGTCGTACT |
| 796 | GGATGGCTACATGATCTGAT |
| 797 | GGCAGCAATTCGGGCTAATA |
| 798 | GCCTAGCAATGTTCCCAGAG |
| 799 | GAGCGGCAATGATGATCCAT |
| 800 | TGGTGCATAGCTGCGATCCA |
| 801 | GGCTGCACAGGTGTATCCAA |
| 802 | GAGATGCCAATCGGCCATAA |
| 803 | TATATGGCACATCGTTGCGA |
| 804 | TGATGCCCACGTCGTCGTAT |
| 805 | ATTGATCCACACACAGTACG |
| 806 | AGCTGATCCAAGCAACGTAC |
| 807 | GTTGATGCAGATCGCGTATC |
| 808 | TCGTGGGCAGATCGCTTCAT |
| 809 | TGTGGCCGAGATGCCTTCTA |
| 810 | TTTGCGGACTTCGCTATCAA |
| 811 | TCCCATGCACCTGAGTGGAT |
| 812 | TTTCATGGAGCTGTCGCGTA |
| 813 | TTTACCTGTGGTGATAGCGA |
| 814 | TTGTCATGCTGCCCAGTCGA |
| 815 | CTTTCATGCAGGCAGAGCCA |
| 816 | CCTTTAAGCTGGCACACGAT |
| 817 | CCTATCAAGGATGCACACGA |
| 818 | CCGTTCAGAATATGACACAC |
| 819 | TAGGTCAGATCATGCGCGAC |
| 820 | ATGTGCATACAAGCTACGAC |
| 821 | CTGAGAATATGAGAGACGCC |
| 822 | ACTCACGCAAATGAACGGCG |
| 823 | CTTAGCGAATATGCGATACG |
| 824 | ACTCTGATAAATCCGACACG |
| 825 | ACTGTGCGAAATCCCAGACA |
| 826 | ACTGATGTAAATCCACACCG |
| 827 | ACGTGAACAATTCCACACTG |
| 828 | ACTGCACGAAATCGACATCG |
| 829 | ACTTCTGTAAATCGCAGCAC |
| 830 | CTGTCTTGAATAGCGATCAC |
| 831 | ATGCGGTTAAGCGGTAATAC |
| 832 | TACGCTGAGTCATCCGAATA |
| 833 | CTTGTGAGACACTCCGACAT |
| 834 | CTGGTGACATACTATCAGAC |
| 835 | CGTGCGTTAAGCTGTCGATA |
| 836 | CGGTATCGAAGCTGTGCTAA |
| 837 | CGCGTGTGAAGCTGCCTATA |
| 838 | CCTAGTAGAAGCTCCACAGA |
| 839 | TGTGTCGGAGTCGCCCATAT |
| 840 | TCTGTCGAGGTAGGCCATAT |
| 841 | GCTGTCGAGAGCGATCATCA |
| 842 | GCAGTCGGACGAGATTCTAC |
| 843 | GCGATGGTACTAGATCAGCA |
| 844 | GTGTAGGGACTCGTATCACT |
| 845 | GTACGAGCAGTTGAGCATAA |
| 846 | GTCAGTCGAGATTCAGCAGT |
| 847 | GTCGAGTCAGATGCACGTCA |
| 848 | GTGTATCTAGCTGCACGCAC |
| 849 | GTTGTCTTACGTGCAGTCAG |
| 850 | TATGTACTCGTATCGACGCA |
| 851 | TCGTGTCGAGTATCCGCAAA |
| 852 | GTACGTTGACAGTCTGCACA |
| 853 | TTCGTAGAGGTCTGCCAATT |
| 854 | ATTCTGAGAGACAAGCCTCC |
| 855 | ATTCTGACACAATCATCGCG |
| 856 | ATTCAGAACTAATGCACCGC |
| 857 | AGGTATGAACCATCGCACAC |
| 858 | ATTTGATGAACTCCGCAGAC |
| 859 | GTTTGCTGACCTCGCAGTCT |
| 860 | ATTGCCGGAACGCATTATAC |
| 861 | TGTGTGGGATCGCCCTATCT |
| 862 | TTGAGTGAGCTGCGCTTATA |
| 863 | TGCGTGCAGGTGCCACTAAA |
| 864 | GTGCTGCATGAGCCAGTTCA |
| 865 | GGCTCTACATGGCGATAGCA |
| 866 | GCTCTCTAATTGCGGACACA |
| 867 | GGATATAAGTTGCGGCACTA |
| 868 | GGATGTAATGGTAGCTCCTA |
| 869 | GGATGACGAGGTCTCACCAT |
| 870 | GGATGCGACGATCTCGACAT |
| 871 | CGTGATCGAAGGCTGCACAA |
| 872 | CTAGATGTAAGTAGCTGGAC |
| 873 | CGAATGAAGGATCGAGACCT |
| 874 | CGGCCTGGAAGTCACTCATA |
| 875 | GGCCTTGGACTACCGCTTAA |
| 876 | TGCTTCGAGGGTCCCACTTA |
| 877 | TGCCTGGTACTGTCCGACTA |
| 878 | TGCTTGTGAGAGTCGCTACT |
| 879 | ATGCTTGCAGAACCGTCAGC |
| 880 | TGACTGTAGGGAGCCTCAAC |
| 881 | TGCTTGGCAGGATGTCTTAA |
| 882 | GGCTCCGGCATGAGTATATC |
| 883 | TGCTTTGCAGTGAGGCTCTC |
| 884 | CAATTTGGAACTAGCCTTCG |
| 885 | TTTGCTGCATCCGGCCTGTA |
| 886 | TTGGGCCACTGCGCTCTTTA |
| 887 | TGTGAGCCCTTGGCACGTTA |
| 888 | GGTGGCCCGATCACATTCAA |
| 889 | GGCAGGGCACCTCAGTTTAT |
| 890 | GGGTGGCCCATGCTATCTAA |
| 891 | GTCTGGCCCTACCTATGGTT |
| 892 | GCGGGCACACCTCTGATTTA |
| 893 | GCGGGCGCACCATTCATTAT |
| 894 | GGAGCCCACCATGAGCTATA |
| 895 | GAATCTCCACCAGGCGGATA |
| 896 | GGATACGTCGCTACAGTGAT |
| 897 | TCGTATAGCTGTATCGACGG |
| 898 | CTAACTAGCTGTAAGCGACC |
| 899 | ACTAGATAACAGATGCGCCG |
| 900 | CAACTATCATCAAGACGGCG |
| 901 | CAACAGAGATGAAGCGCGTC |
| 902 | CAACATATCATAAGCGCGTC |
| 903 | GCAGATAGCATCATATACGC |
| 904 | GCAGACTGAATTAGCTCTAC |
| 905 | GTTAATTCATCTAGCGCGAC |
| 906 | AGGAATCTAACCACGCGCAG |
| 907 | AGACCAATAAGCACCCTGGG |
| 908 | AGACAAACATTCACGCCGGG |
| 909 | AGAATAAATTACTGCCCGGC |
| 910 | GAGCACATATTATTACGCCC |
| 911 | CAGAAGATAATATGCTCGCC |
| 912 | GAATAGCCGATAATCTCAGC |
| 913 | GAATAGCTTTACACTGCCCT |
| 914 | GAATCACTCTGAATGAGCAC |
| 915 | GGATCACACTGCCGGACTAT |
| 916 | GGACCCATAGCACTCTGATT |
| 917 | GAGGCATTAGCACCAGCTCT |
| 918 | GGATTATCAGCACTCAGTAC |
| 919 | GGGATCTCAGACGATGCTCT |
| 920 | GGGTATATCAGGGGATTCCA |
| 921 | GCAATTCGATCTAATGCTCC |
| 922 | ACCAATGCAAATAGGCGGCC |
| 923 | AGCAAATTAACACTTGGGCC |
| 924 | GAAACAAGCAGATTTGCGGC |
| 925 | TTAATTCCGTGATATGCGCG |
| 926 | GGATCTAATGGTTATGACCG |
| 927 | GCATGAAGTGGTGTCAACTC |
| 928 | GCTTTAATGGTCGTGACGCC |
| 929 | GCTTAGAATTTAGTGCAGGC |
| 930 | GCGTCAGAATTTATGCCACA |
| 931 | GCTAGATAATTTAGGCCACG |
| 932 | GCTGATAATGCTGAGGACTA |
| 933 | GCAGAATTGCATAGACGCAC |
| 934 | GCATGATTAGCATAGACGGA |
| 935 | CCAGCAATAGGAATCACGGG |
| 936 | ATTGCACATTCAACTGACGC |
| 937 | TGGCATTTACTTAGTGCGAC |
| 938 | GAAGCCATATCAATGCTCAC |
| 939 | GCGAGCAATTTCATGCCACT |
| 940 | GGCCCAAGTTTGTGACATGA |
| 941 | GGGCATAATGGTTGATACTC |
| 942 | TTGGTGCATGGATCTCTCCC |
| 943 | TTTAGGGCAGGTTAGCTTCC |
| 944 | TTATCCGGCTAGAGTGCGTC |
| 945 | TGATGACCTGTTAGCAGTAC |
| 946 | GGACCATGTGCTACGCAAAT |
| 947 | GTGAGCAGATTCAGCCAGAC |
| 948 | GAGAGACCATGCAGCCGATA |
| 949 | GCGTCGTCAATGTTGCCACT |
| 950 | GGGTTAATCCCTGCCACGTA |
| 951 | GTGCTGACATTCGCGCCATT |
| 952 | GCCTGTAATCGTGGGCACAT |
| 953 | AGCGCGTGAAATGCACATAC |
| 954 | AGCGTCTGAAATGCTATCAC |
| 955 | AGTGCGCGAAATGTTCTAGA |
| 956 | CGTCGCCAATATGATCGAAT |
| 957 | CGCCACAAGTTCGAGCGATA |
| 958 | GCCCTACAGCGTGAGCTATA |
| 959 | TGTCAGTGATCCGGGACTAT |
| 960 | GTTATCGCACCTGAGGCGTA |
| 961 | GTTGTGACCTCTGAGCACGT |
| 962 | GTTTCACGCTATGCGAGCCA |
| 963 | GTTTACCGCTCTCCAGGGAT |
| 964 | TGCGTACCTCCTGCATGGTT |
| 965 | TGACTACCGTGTCGCATACG |
| 966 | TGGACTACGTGTCTCGATAG |
| 967 | TAGTGATACTGACTCATGGC |
| 968 | CGTCTGATACAGCCCAGTGT |
| 969 | GCCGTATCACGACGCTAGAT |
| 970 | AGCTCGATACAACGCTAGAG |
| 971 | ATCTACTTAACGCGCTACAG |
| 972 | GACATCGTACCACTGCGTAG |
| 973 | GACTCGTGACCACTCTGTAG |
| 974 | GACTCGGACCATATCTACGG |
| 975 | CACTACGCAAGACTATGTAC |
| 976 | CGAGTCTCACAGCAATCTAG |
| 977 | CGATCTAGCACGCAATATAC |
| 978 | GACCAGCGACGACAGTAGAT |
| 979 | CGTAGACAGCCACGCAGTTA |
| 980 | CGTATGCTACCACCGATTAT |
| 981 | CGTGCGATACCAGCGTAGAT |
| 982 | CTCCGTACAGCAGGCAGTAT |
| 983 | CTCGTCGTACAGCGATCAGT |
| 984 | CTACAGATACGTCGAGAGAG |
| 985 | CTACGCGACACGCATGAGAT |
| 986 | TAGACGCTCGCACGGTAGTA |
| 987 | GCCGCTAGACGACGGTATAT |
| 988 | GTATCACTAGGACGAGGTAT |
| 989 | GTACTCACAGTGCGAGAGCT |
| 990 | CGACTACACAGCTCAGGATA |
| 991 | CACCGACAACTCGTAGAGAG |
| 992 | CGACCCACACTAGGAGAGAT |
| 993 | ACGCGCACAACAGGAGACTT |
| 994 | AGTACCACAACTCAGACGTG |
| 995 | AGTACAGCAACGCAGAGCCT |
| 996 | GTCAGCGACCGTCAGCTATT |
| 997 | GTCAGGCACTAGGAGCTATC |
| 998 | TGTCGGTCACTCCTGGACTA |
| 999 | TCGGTTCACGTCCGCATGTA |
| 1000 | TCGTTTACCTGTCGCGCTGA |
| 1001 | TGTGTCTCACTTCCGCGAGT |
| 1002 | TCTGAGCACTCTCTCGTAGG |
| 1003 | GTTGATGACTCGCCACACGT |
| 1004 | CTGAGATCACAGCAGACTAG |
| 1005 | TTAGACTCCTCGCCGGTAGA |
| 1006 | TATAGCTCCTAGCAGGCGTA |
| 1007 | TATGCTCCACGTCTAGTGAG |
| 1008 | CTCTATCACCAGCGATGAGA |
| 1009 | CGCTCCAGACAGCATATAGA |
| 1010 | ACATACCGAAAGCTCTAGCG |
| 1011 | ACATCGCTAAAGCACATCGG |
| 1012 | ATATCGCGCAATCAACGCTA |
| 1013 | CGATGCGCCACTCAAGGTAT |
| 1014 | TATGCCGACGGTCAGGCTAA |
| 1015 | TATCGCCACGTCCGGTGATT |
| 1016 | TCTCGCTCACTGCGTATGAT |
| 1017 | TATCCGTCACTCCGTAGAGG |
| 1018 | TATCGACTATCCCTGAGACG |
| 1019 | GTATAGACCTCTCAGACGCG |
| 1020 | CTATCGTAATATCAGTCCGC |
| 1021 | CGATGACAATTAGGTACACG |
| 1022 | GAGCATAATGACGTAGACCG |
| 1023 | CGACAATACTTGACAGCACG |
| 1024 | CGATGATAATAGAGTAGCCG |
| 1025 | CTATGATTAAGTCGTAGCCC |
| 1026 | AGGTGAATAACGCATACGCC |
| 1027 | GAGTGAGTAATGCTACGTCA |
| 1028 | GATCGACGAATGTTAGAGAC |
| 1029 | GACTCACGAATGCGGAGACT |
| 1030 | GACCGTCAATCGCGTCAGAT |
| 1031 | TACCCGCATCGACGGAGTTT |
| 1032 | GTCAGCGCACTCCTGGTTTA |
| 1033 | TCAGGCCCACGTAGCGTTAT |
| 1034 | TTCGCGCTATCCATGCGTGA |
| 1035 | TGCTGATACTCGGCTGCATC |
| 1036 | TGAGTAGCATCGGTGACTTC |
| 1037 | TTGTATCACTGTGCTGCCCA |
| 1038 | TTTAGTCAGTATGCTCGCGG |
| 1039 | TTACGTTTATATGGCCGAGG |
| 1040 | TGAGATCACGTTCGCCGAGT |
| 1041 | GTATCATTAGCTCCGCAGAG |
| 1042 | TATCATGTAGACTCGGAGGC |
| 1043 | GTATGCTTAGATATGCAGCG |
| 1044 | TTGTAGTTAGCTCTGCACGG |
| 1045 | ATATCGTTAAGCCATACGCC |
| 1046 | ATTCTGATAACGCTCTCGAC |
| 1047 | ATTCGTCCAACGCGGTCGAT |
| 1048 | ATATGCACAACGCGCAATCG |
| 1049 | TTAGCTCTATCGCAGTCCGA |
| 1050 | ATTAGCTGAACGCCTCGCAA |
| 1051 | ATTATCTCAACGGAGGAGCA |
| 1052 | ATGTTGCTAACGGACGGACA |
| 1053 | ATGTGTTCAACGGAGACAGA |
| 1054 | CTCTTTCTAAGTGAGTCGAG |
| 1055 | CTGCTTGAAGTCGTCTCACG |
| 1056 | CTGCGTTGAAGTGGCTTACT |
| 1057 | GTGCGTTCACATGGCCGTAT |
| 1058 | GTAGCCGCACCTGACTGTAT |
| 1059 | GTAGCGCCACCTGACGTTAT |
| 1060 | GGCGCGTCACATGATACATT |
| 1061 | GGTTGCTACGATGACTCAGT |
| 1062 | GAAGGCCCGTACACTCTATA |
| 1063 | GACAGGGCACACGACTCTAT |
| 1064 | TGCGCGGCACTCGTTCTATA |
| 1065 | GCGGTTGCACTCGTAGCATA |
| 1066 | GAGGCGTGACCAGTCCATAT |
| 1067 | GGACGCTCACCAGTGCTTAT |
| 1068 | AGTGTCCAACCAGACCAGAG |
| 1069 | AGTGCCATACAAGCGCATAG |
| 1070 | GTAGCCTTACATTGGCAGAG |
| 1071 | GTCGCCGCACATTCGGTTAT |
| 1072 | GTTGAGTCAGATTAGCAGTC |
| 1073 | TCGTAGGGACTGCGCTCATA |
| 1074 | CTCAGATGACAGCGACGCAT |
| 1075 | CTCTGAGGACAGCCGAATCT |
| 1076 | CTAGGATGACAGCCAGACAC |
| 1077 | CGTGAATTACATCAGACAGC |
| 1078 | CTGATTATAGCTCATACGCC |
| 1079 | CTAATATGATGACAGTCCGC |
| 1080 | TACTTATGATGACTGCGGAC |
| 1081 | GAACTATGCTGACAGTACCG |
| 1082 | CGATTCTGACCACATACGAG |
| 1083 | CTAATCTGACCACGAGACGA |
| 1084 | CTGTATTGACATCAGACGAG |
| 1085 | CTTCTCAGACATCGGACGAG |
| 1086 | GCACTGTGAATTAGCGAGCA |
| 1087 | GCCTACGGAATTGGCAGACT |
| 1088 | GACCTGGAATTAGCACACGC |
| 1089 | GCCTGCGAATTAGCGGACAT |
| 1090 | GCGATGCTAATGATGTGTAC |
| 1091 | GCCCGTCTAATGAGTGGACA |
| 1092 | GCCTAGCTCATCAGACGGAA |
| 1093 | GCATGGACATCCTACGAGAA |
| 1094 | CGCCTGCCAAGCTGTGATAT |
| 1095 | GCCTGCGCCATCAGTAGATA |
| 1096 | GCACGGCCAATTACTCGATA |
| 1097 | GCAGCGAGACCATGTGATAC |
| 1098 | GCAGCAGCACACTGATCGTT |
| 1099 | GACCCAGCACATTAGCGAGA |
| 1100 | GCTCCTGCAATGTGCGGATA |
| 1101 | GCGCCTGAATTGTAGCACGT |
| 1102 | GCCACAGCATTGGAGAGAAT |
| 1103 | GCCAGGCTAATGGATAGTAA |
| 1104 | GCCCTGCGAATGAAAGACAT |
| 1105 | GCAGCGGGAATTAGATATAC |
| 1106 | GCAGGTGCAATGATTCTACC |
| 1107 | GACCGGGCAATCACTTCAGA |
| 1108 | GCCGGGCAATGCGTTCATAT |
| 1109 | CCCAGGGCAAGCGATCATAA |
| 1110 | GCCACAGGCAGGGCATATTA |
| 1111 | GCCTAATCCTGGGACACTGA |
| 1112 | TCGTCTCGATCTAGGCCATG |
| 1113 | GTGTCTCGACTCAGCCTATA |
| 1114 | GACGTAGTAATCATGTCTCC |
| 1115 | GACTTATACGTCATGCGACC |
| 1116 | ACGATGTAACACAGCGACCG |
| 1117 | AGTCGTGTAACCATGTGACA |
| 1118 | GTCGTGACAGTGATGTACTC |
| 1119 | GTGGAGTGACGTATCTCTAA |
| 1120 | TAGAGGTGACGTAGTCCACT |
| 1121 | GTCGTGCGAGATAGCTCTTA |
| 1122 | GTGTAGAGATATAGCATCGC |
| 1123 | TAGTCGTGAGATAGCGATTC |
| 1124 | CAGTGTGTACGAATACGAAG |
| 1125 | CGAGTGTCACATACCACATA |
| 1126 | CGTATAGCAGACAGCGCAAT |
| 1127 | GACATCGACGACAGGCCATA |
| 1128 | CGAAGCTCACGTAAGTCAAG |
| 1129 | TAGTGCTCACGTAGCCCAGT |
| 1130 | TGCCCACGGTGAGCTAGTTT |
| 1131 | TAGCTGCCAGGAGCGTTCTA |
| 1132 | TCGGCCTACGCTGTGCATTA |
| 1133 | TAGGGTACTGATGAGCACTC |
| 1134 | CTACGGGAAGGTTAGCACCA |
| 1135 | TGGTGATACCTGTGCGCCTA |
| 1136 | GATTAGATACCACTGCCACA |
| 1137 | GGAGTGATACCTCGATCCAC |
| 1138 | AGCTGACGAAATCTTCACAC |
| 1139 | GAGGAGATAATGGTCACTAC |
| 1140 | CACGGAATAATACATCCTCG |
| 1141 | ACAGCAACAAGTCGAGCCGT |
| 1142 | ACGGAGAGAAATCAGCCCTC |
| 1143 | CAAGAGATAATACGGCTGCC |
| 1144 | CAAGTCCTAAGACAGCTACG |
| 1145 | ATAAGCGCAAGACAGGCGTC |
| 1146 | ATCTGAGCACAACTAGGACG |
| 1147 | CACAGGCTAAGACAGGAGCT |
| 1148 | CATAGCGTAAGCCAAGCAGC |
| 1149 | CATAGTCTAAGCCACATCAG |
| 1150 | GACAGTACATGCCAATCAGC |
| 1151 | GCGGTAATCGGTGCATCAAA |
| 1152 | GGGAGTATAGCTGACCATCA |
| 1153 | GTAGGCAGACCTGATCCCTT |
| 1154 | GAGCCAGACCACGCTTGATT |
| 1155 | GGCGCATCACTAGCCAGATT |
| 1156 | GGAGCTACATCCGCCAGTTT |
| 1157 | GGAGTCTACCCAGGGGATTT |
| 1158 | CGCGCTCTACACGATGGATA |
| 1159 | CGTGCCACACCTTGGAGTAT |
| 1160 | CGCGGCACACAGTTCAGTAT |
| 1161 | GCTCGTCCACAGTGCGTTAT |
| 1162 | GCTGACGCAGAGTCCAGTTA |
| 1163 | CCGTAGCGACAATCAGCTTA |
| 1164 | ACGCACCGAAAGTGAGCGAT |
| 1165 | ACGTCCTCAAAGTGCAGACA |
| 1166 | ACGCAGTCAAAGTCATATCC |
| 1167 | CAGAGTCTAAGATCACCACG |
| 1168 | CACTGTCTAAGATACACACG |
| 1169 | CAGCGTACAAGCTATACAGC |
| 1170 | CCGACGACAATGTACGACAG |
| 1171 | GACTAGCGAATCTAATGAGC |
| 1172 | CGTCGAGCAATATGAATGAC |
| 1173 | CTGTCGCGCACTTCATAGGA |
| 1174 | CCGCGACCACGATAGAGAAT |
| 1175 | GGCACACACGTCTCGGATAA |
| 1176 | GGCAGACGACGTTGCATACA |
| 1177 | CGTGGGACACAGTCGATCAT |
| 1178 | AGTGCGAGAACATCGTGTAA |
| 1179 | GGCAGCACAGCTTGTACGAT |
| 1180 | GACCATTGAATATGTCGAGC |
| 1181 | GTACGCATATTTAGCCAGCA |
| 1182 | GGCAATCTGTTCACGACCAA |
| 1183 | GCTGACTAATTGCTAGACAG |
| 1184 | GGTGTCTAATTGTATGCAGG |
| 1185 | GTTGACACATTGTTAGCAGC |
| 1186 | TTAAGAGATTAGTCTGCCGC |
| 1187 | TCACGTAATTTGTTAGCCGC |
| 1188 | TGAGTGATAGCTCGGATCTC |
| 1189 | ATGATGATAACTACGTGCCC |
| 1190 | ATGCGAATAACTATGACGCC |
| 1191 | ATGGAGATAACTATGCACCC |
| 1192 | TCGTTGCGACCTATGCGTAG |
| 1193 | TAGTTCGCACCTACTGCTAG |
| 1194 | ATACGTGCAACCACTGCTAA |
| 1195 | ATGTCGATAACCTCTGCTAC |
| 1196 | ATCTAGTCAACCTGAGCTAC |
| 1197 | AGTATAGCAACCTCAACTCG |
| 1198 | AAGACACTAAACTCTGCTCG |
| 1199 | ACGATAATAACAGCTCCTCG |
| 1200 | ATAGATATAACTGACGCGCC |
| 1201 | ACTGTAATAACCAAGCCTCG |
| 1202 | ACTGATAGAACCACAGCGCG |
| 1203 | ATGGCGACACACATACAGCG |
| 1204 | ACGGCGAGAAATACGATGCC |
| 1205 | GACGCGAGATCAATGTAGTA |
| 1206 | CGAGAGTAATCAATCATCCG |
| 1207 | CGAGCAATACATACATCTGC |
| 1208 | CAACATAGTTACACACGCTG |
| 1209 | CAGCTTATAGAGACACACTC |
| 1210 | CCATAGAAGTAGACACCTCG |
| 1211 | CTCAGAGACATGACACTCGA |
| 1212 | ATCAGGTCAACTAATCACCG |
| 1213 | AGCGCAGTAAATAGCTTAGC |
| 1214 | ACTCCACGAAACATGATTGC |
| 1215 | CTCAATATAGACACGATGCC |
| 1216 | CGCATTAGAGACAGATCGAG |
| 1217 | CGCACATGACATAGAGCACG |
| 1218 | CGCACATTAGACAGAGAGGC |
| 1219 | CTAGACTAATGCAGAGAGCG |
| 1220 | GCGTATAGATGCAGAGATCC |
| 1221 | TCACTAGCGTGGAATAGAGC |
| 1222 | CAGACTGAACTCAATGTACC |
| 1223 | CACGATGAACTAGATGTACC |
| 1224 | CGAATGATAAGTATGACGGC |
| 1225 | CGAGATGCAAGTATAGTACC |
| 1226 | GGATAGCGAGATATAGACCC |
| 1227 | GCATAGCACGATGGACGATC |
| 1228 | CTCACAGGACATGCAATCGG |
| 1229 | TATACATGCTTCGATCACCG |
| 1230 | ATATCAATAACTGCGACGCC |
| 1231 | AATACGAAAGATGCGGCCCG |
| 1232 | ACAGATACAAATGTCGCCCG |
| 1233 | ACGAATAGAAATGTGGCCGC |
| 1234 | ACATTACTAAAGGTGCGACC |
| 1235 | AGATTAGTAAATGCTGCGCC |
| 1236 | ACTATGATAACAGCAGCCCG |
| 1237 | ATATGAATAACTCCAGCGCC |
| 1238 | AGACTGAAATCTACAGCCCG |
| 1239 | GTACTGATAATTGGATCGCC |
| 1240 | CCAGAACGGTTGCAGACACT |
| 1241 | GCAATAGTTGGACCCAGGCT |
| 1242 | GGAATAGGTGGACTCACTCA |
| 1243 | GCACAAGTTTCGCGCATCGA |
| 1244 | GCGGAATCTGTGCAGCATCT |
| 1245 | GCGAGAATATGGTGACATCT |
| 1246 | GCGGTCAATTAGTGGACTCC |
| 1247 | CTCCTACAATGGTGACACTG |
| 1248 | CTATTACAATGGTATGCCCG |
| 1249 | AATCATACAAAGTGTGCCGC |
| 1250 | CATGATCTAAGAGTGTAGCC |
| 1251 | CAAGAAGTAAGATGCGTGCC |
| 1252 | CATGTGATAAGATGTGGACC |
| 1253 | AACTTAGCAAACTTAGCGCC |
| 1254 | TCTTCGATATGATAGCGTCG |
| 1255 | GACGTTAATTGATGAGACGC |
| 1256 | GCGTGAAGTTGTTAGCACAT |
| 1257 | GCCGATACATGCTGCACGAT |
| 1258 | CGCCGATTAAGCTGCGACAT |
| 1259 | CGTCATTTAAGTTAGCGCAC |
| 1260 | CTCCATCTAAGGTGCGATAC |
| 1261 | CGCTTATCAAGGTGCAGACC |
| 1262 | GATGACTCAATGTGACTCAG |
| 1263 | CGCTAGTGACAATTATGTGC |
| 1264 | GCTAGGTGACAGTATGCTAT |
| 1265 | GCTGTGCTACGACGTTGACA |
| 1266 | GCTAGAGTAGACCGATGCCA |
| 1267 | GTATATCGAGATCATAGGCG |
| 1268 | GTCTTGGACTATACGAGCGC |
| 1269 | TACTTGTAGATAGCGAGCGA |
| 1270 | GTACTCTGACATGATTCGCA |
| 1271 | TATACTGACCTTATCGGCAC |
| 1272 | TCGTCTTGAGATATGTGGAC |
| 1273 | TCATGTTACGGTATGCGAGA |
| 1274 | TCATCTGCACGTATCGTCAA |
| 1275 | GCGACTGGACAGATTGCATA |
| 1276 | CGGGCGCGAAGTATTCACAT |
| 1277 | GTGTGGGCACGTATTCCATA |
| 1278 | TCCGGGCACGGTGTCATATA |
| 1279 | TGGGCGCTACTGGCTCTTAA |
| 1280 | TGCGCCGCCAGTCTGTTATA |
| 1281 | TGGCCGTTAGAGTCTGCACT |
| 1282 | ATGGGCGCAACCCTGTCATA |
| 1283 | CAGCCCTGAAGACTGCGATA |
| 1284 | CGCCGCTCAAGGCTATGATA |
| 1285 | CGCTCCTGAAGGGTAGTTAA |
| 1286 | GGCCCGACAGGTGCTATTAT |
| 1287 | GGATAGGCAGATGCACTTAT |
| 1288 | GGACAGACGTTGACCAGCTA |
| 1289 | GTAGCGACATTGAGTTAGCA |
| 1290 | GACTACGAATTGAGCATACG |
| 1291 | CTACACTAATTGCAGCAGCA |
| 1292 | CGTACCCGAATGCAGCAGAA |
| 1293 | GACGCCTAATGACGCTGAAA |
| 1294 | TAGCTTGTACTGCGACTGAC |
| 1295 | GATACTCTAATGCCATCGAC |
| 1296 | CGGCGTACAATGCCATAGAA |
| 1297 | CGGATACGAAGGCTATGCAA |
| 1298 | ACGGATCGAAAGGTATAGCC |
| 1299 | ACGGCGCGAAAGCGTCATAA |
| 1300 | CGTGAGGGAATACGTCATCA |
| 1301 | CACAGTGGAAGACGCATCAC |
| 1302 | GAGGTGACATGACGTACATC |
| 1303 | GAGTAGCGAATGCTCAGCGA |
| 1304 | TATAGCACAGTGTCCAGCAA |
| 1305 | CGTATGTCAAGGGCCTGATA |
| 1306 | CGAGACGCAAGGGATTTACA |
| 1307 | GAGACGCAATGTGAATTACG |
| 1308 | GATCGCACAGGAGCGTATCA |
| 1309 | TGCCCAGAGCGTATGAGCAA |
| 1310 | TGAGGGCGAGCTATCTATCA |
| 1311 | TTGTGGCTAGGTATCGCTAC |
| 1312 | TGGTTAGCAGGTATGATCCT |
| 1313 | CTCACTGCAAGGATGGGACT |
| 1314 | TCCTGTAGATCCCTATGCGG |
| 1315 | TCGTTGTCAGCATATTGAGC |
| 1316 | ATCATGTGAACCTATTGGCC |
| 1317 | TACACTGGGACCTATGGGCA |
| 1318 | TACCTGGGAGCATAGCTGAC |
| 1319 | TAGCCCGCAGCATAGGGTAT |
| 1320 | GAGCCTCAATGCTACGGAAG |
| 1321 | GATGTTCAATGCTGGCCGAA |
| 1322 | GACTTGTGAATATCTGTGCC |
| 1323 | GCCGCCGAATTATTGAGCAA |
| 1324 | TGGACTGATTGATAGGCAAC |
| 1325 | TGGCAGATCGGTGTATTCAA |
| 1326 | TATGCGTAATGGGTGTTCCA |
| 1327 | TTAGGTCGATTGATAGTCGC |
| 1328 | TCTGCTTTACTGCGTAGCCA |
| 1329 | TTGACGAGTTTGCAGTGCTC |
| 1330 | CTTGATTAAGTGCTGTACGC |
| 1331 | CTCGGATCAAGGCTTACCGT |
| 1332 | CCGGGCTCAACGCTTTGTAA |
| 1333 | TGTCGCCCAGCTCATGTGTT |
| 1334 | CTGGACCCACAGCTATGGAT |
| 1335 | CACGGGCCAAGAGATATACC |
| 1336 | CGCCCGCCAAGTGATGTATA |
| 1337 | CGCCAGCCACATGGATAGAT |
| 1338 | GCCCGGATACATGCGATTAG |
| 1339 | GCTGGCCTACATCCGTATGA |
| 1340 | AGATGGCGAAATCCGTATAG |
| 1341 | GCAGGGACATTACGATCAGT |
| 1342 | AGCAGGTGAAATCGTACTAC |
| 1343 | GCAGGTCAATCTCTGTACGA |
| 1344 | GCATTGTAAGTTCGGTCAAG |
| 1345 | GCACTGGTAATTCAGCTACG |
| 1346 | AGCATCATAACCCAAGCTGG |
| 1347 | ACCAGTCCAAAGCATAGTCG |
| 1348 | ATCATTTCAACGCAGTGACC |
| 1349 | TCAGCCCTATCGCAGGATGT |
| 1350 | GTCAGCACCAGCCGTGATTA |
| 1351 | GAATTACGCACCCAGCTTGA |
| 1352 | GAATGCGCCTACCAGCTATA |
| 1353 | GAATGGCGACAGCGTACATA |
| 1354 | GGATTGCCACGACTCACAAA |
| 1355 | GCTCATTGACACTGCGCTAT |
| 1356 | GAGCATGGACCACGGCTATA |
| 1357 | CAAATGGACAGACAGCCTGC |
| 1358 | CACTTTGAAGCACAATCACG |
| 1359 | GCTGTTGCAGGACGCATCTA |
| 1360 | TACCTGGCATGACGCGATAT |
| 1361 | TTCGTGGACTTGCGGATCTA |
| 1362 | TTCCTGCGATAGCGGCGTTT |
| 1363 | TTGATCTGATAGCGGGTCTC |
| 1364 | TTGATCGCATAGCGTCTGAC |
| 1365 | TTCGAGGCATGTGGATCTCC |
| 1366 | TTCAGCGGCTAGGCGATTTC |
| 1367 | TCCAGCAGATCGGCGAGTTT |
| 1368 | TTCAGCCGATCTGCCGATAT |
| 1369 | TTCTATCGCATGTCAGCCGT |
| 1370 | TGTAATGCCTGCCAGCCGTA |
| 1371 | TAATTGCCTGCACAACTGGA |
| 1372 | TAATTCCATTGACGGCAGCG |
| 1373 | TTATTGCCATAGCGCGACGC |
| 1374 | ACAATTTCAAAGCCTGACCG |
| 1375 | ACAGGCCCAAAGCACTAGGT |
| 1376 | CGAATGCCAAGGCCAGCTAA |
| 1377 | GATGGTTCAATGCCTGGACA |
| 1378 | CTGGGCCAAGTTCTGAGACA |
| 1379 | CGTGGGCAATACAGTTGAAT |
| 1380 | GAGCTGCGAATCGGTATTAA |
| 1381 | GACCGGCGAATCGAGCATAA |
| 1382 | GACTTCGCAATCGGCACGTA |
| 1383 | GACGCGCCAATCGTGCTATA |
| 1384 | GATCGCTGAATCGTGCGTAA |
| 1385 | GATCACTGAATGCGACGTAA |
| 1386 | GATCGTGCAATGAGGTTACA |
| 1387 | GAGGACTAATTGAGATGCAC |
| 1388 | GACCGATAATTCGATATGCC |
| 1389 | TAGCATTGATCCCATGTCAC |
| 1390 | TTCAGCTTATGCCAGTCGCG |
| 1391 | TGACGGCCTTGCATATCCGA |
| 1392 | GAACGCGCCTTACATCAAGA |
| 1393 | GAATACCAGTTACACTCCAG |
| 1394 | CAAGAACTGTTACACATCGC |
| 1395 | GACGAGAATGGACTACACGT |
| 1396 | TACAGACGCTTGCATAGATC |
| 1397 | TAACGACCTTAGCGACGGGT |
| 1398 | TAACGACGCTTTCCCAAGGA |
| 1399 | TTACCGCTGTTGAGCCCGTA |
| 1400 | TTCCATGTATCGAGCGTCAG |
| 1401 | TATACGCCCTTCAGATCGGG |
| 1402 | CTAAGCCTATGCAATATCGC |
| 1403 | CCAGCTATAAGCATATTGCC |
| 1404 | TACAGCATTGTCATGGACTC |
| 1405 | TAAGCTATTGGACATTGGGC |
| 1406 | TTAGCATCCTGTCATAGGGC |
| 1407 | TCTAGCAGCTTTCATAGCCA |
| 1408 | TCATCACGCTTTCCGAGGAT |
| 1409 | GCATACATTGGACGAGAGCT |
| 1410 | TCTAGCATTTAGCATGGTGC |
| 1411 | TTATGACTTGATCTGAGGCG |
| 1412 | TGTTCGCACTGGCTTAGCTC |
| 1413 | GAGTTGAATGCAGATAGCTC |
| 1414 | TGCAGGCTCGCAGATGCTAT |
| 1415 | TGCGAGGACTGTAGCTTAAT |
| 1416 | TGGGCACTCTCGCCTAGTTT |
| 1417 | TGAAGCGCCTCGACTAGGTT |
| 1418 | TCATCGGCACTGATAGCTCA |
| 1419 | TCATCAGGCATGGAGCCAGT |
| 1420 | TAATCAGCGTTACGTCCGCA |
| 1421 | GAATGTGACGCAAGTCTGAC |
| 1422 | AGATTTGCACAGATAACGCG |
| 1423 | GATTACTGACCAGCATCGAG |
| 1424 | AACTATCGAAACCGCCAGGG |
| 1425 | ATAATACAAGAGTCGCGCCG |
| 1426 | ATAATCATAACCTCGACGCG |
| 1427 | ATTATCATACAAGGCAGGCG |
| 1428 | TATATCGGATCAGCAGGTCA |
| 1429 | TAATTTCGCTACGCAGGGAG |
| 1430 | TAATCCTGTTACGCGGAGGC |
| 1431 | CTTTAGCTCCACGCAGTGTG |
| 1432 | TTCTAGCCGTCCGCAGTTTG |
| 1433 | GTCATGCGAGCAGCAGTCTT |
| 1434 | GGCGTTCGAGCAGTCATCTT |
| 1435 | TACCGCCAGTCAGCGAGTTA |
| 1436 | TACCGCCTAGCAGCATTGGT |
| 1437 | TACCGCACTGCATGTCAGGT |
| 1438 | TGTCTCGATGCAGGTCTAGT |
| 1439 | GCCGCATGACGAGGATATAC |
| 1440 | TACCGCGAGGCAGGATTCTT |
| 1441 | TACAGCAGTGCAGGGCCTTA |
| 1442 | GCAGCTAGAGCAGAGTATCA |
| 1443 | GACAGCAGATCAGAGACTCC |
| 1444 | TAAGCACGTTTAGAGCTGAC |
| 1445 | TAACCGTGTGCAGATCGGAT |
| 1446 | TACTGCGGACCTGGATCTAC |
| 1447 | TCAGGGCTACTCGATTGGAA |
| 1448 | TCCGCAGACTTAGCGTTACG |
| 1449 | TGAGCAGCCTACGTTACTAG |
| 1450 | TGCGTCAGATGCGTATATGC |
| 1451 | TCGTCCAGATGCGGAGTTCA |
| 1452 | TCGGCTATATGCCAGATCCT |
| 1453 | AAGGACAAAGAGCGCGTCTC |
| 1454 | TAGCACCGATGGCGAGCTTA |
| 1455 | TGTCCACGGTGCCGCAATAT |
| 1456 | TGGTCCGACTGCTGCTACTA |
| 1457 | TGTGCCGACTGCCGTCTTAT |
| 1458 | TTCGCAGTATGGATCGGTAT |
| 1459 | TTACGCAGTTGCATGGAGCT |
| 1460 | TTCTGATTAGCTGCGGACGC |
| 1461 | TGGTTATACTTTGCGAGAGC |
| 1462 | TTTGTTAGCTTCGGGCAGCC |
| 1463 | TTGGTCTGATCCGGGCATAC |
| 1464 | TGCTTGGACTCCGGCGATTA |
| 1465 | CTGCTTGGACCAGCCAGTTA |
| 1466 | AAGCTGGGAAACGCACACCT |
| 1467 | AAGCGGGCAAACGATATGCT |
| 1468 | AAATGCCGAAACCATCTCGT |
| 1469 | CCATTCGGAAGCGACTCGAT |
| 1470 | TACATGGGCTGAGAACGCAA |
| 1471 | TATTGGGCACGAGCGCCTAT |
| 1472 | CATCCGGGAAGAGTAGCACA |
| 1473 | ATTTCATGCACATAGCACGC |
| 1474 | ATTGCAGCACAAGCCAGACT |
| 1475 | TTGCTAGGCTCAGTCCCGAT |
| 1476 | TTGGCGAGCTGCGTTCTCAT |
| 1477 | TCCCAGAGATGCGACTGCTA |
| 1478 | TCGCTGGATCGGCATGTCT |
| 1479 | TTGCTCCTAGCTCGCGTGAT |
| 1480 | TTGCTGCTAGTCCAGTAGGC |
| 1481 | CATTAAGCAGTCGAGAGACC |
| 1482 | CGTTAATGCAGCGAGAATCA |
| 1483 | CGCAAGCTCAGCAGAATTAC |
| 1484 | CCATGTCGAAGCATTCATAC |
| 1485 | CTGAATGTAATCATCGTGCC |
| 1486 | CTTAGATGAATCACTGCCAC |
| 1487 | CTTCACGGAATCTAGGCACA |
| 1488 | CACTCTTGAAGCTAAGCACA |
| 1489 | CCTCTAAGCATGTTGACACA |
| 1490 | CATGCCGGAAGATGCGTACA |
| 1491 | CAGGCAGCAAGATGTACGAC |
| 1492 | CAGTGGGCAAGATAAGATTC |
| 1493 | CCGTGCCCAAGCTAGTGATA |
| 1494 | GATCGGGCAATCTGCGTACT |
| 1495 | TTCAGTGCATTATAGTGCGG |
| 1496 | TTATCTGCATGAGTAGGTCG |
| 1497 | TCGATAATCTTTGTAGCGCG |
| 1498 | TCTTACAGCTTTGCAGGGAG |
| 1499 | TCCTACATTTGCCACGGGAG |
| 1500 | TCTTCATCAGTGAGGCGCGA |
| 1501 | TTTCTAGGATGTATGCGAGC |
| 1502 | TATCCAGCATTACTGCGAGA |
| 1503 | TTATTCTCAGCACGCACGGA |
| 1504 | TGATTCGCACTCGCGGCTAA |
| 1505 | TTTGTATGAGTCGCTCCGAA |
| 1506 | TTCCGATCAGTCGATGCAAA |
| 1507 | GATCGTCAATCTGATGCACC |
| 1508 | AGATCGCTAAATGAGGACCC |
| 1509 | GATGCTATAATCGTATGGCC |
| 1510 | AGGAGCGTAAATTATCAGCC |
| 1511 | GGGCGATGACTATATCTGAA |
| 1512 | CTGGATTGACACTAGCATAC |
| 1513 | CTGCGGATACCATAGACAAC |
| 1514 | ACTGCAATAACATATCCGCG |
| 1515 | AATGACATAAAGTGCTGCCC |
| 1516 | ACATGCAGAAAGTAGTCCGC |
| 1517 | ACAGGCGAACAATGTACCCG |
| 1518 | ACCAGCACAAAGTCTACTGT |
| 1519 | AGAGAGCCAAATGACTGTCC |
| 1520 | TAGTGCATAATTGCTTGCCC |
| 1521 | TGAGCATATAGTATTCGGGC |
| 1522 | TGAGCGTTAGAGCTTGATCC |
| 1523 | TAGGCGCTAGGACTCGTTAT |
| 1524 | TATGGCCGACGATGTGTCAC |
| 1525 | TATGGCTGACGTAGCGCACT |
| 1526 | TCTCGGTTACTGAGTGGACT |
| 1527 | ATAACGGGACAGAAGCTGCT |
| 1528 | ATAGAACTCAATAGCCGCTC |
| 1529 | CATAATACACATACGCTGCG |
| 1530 | CAGTACGCAAGCAGATAGCC |
| 1531 | CAGACGCGAAGATAAGTTCC |
| 1532 | CAGCCAAGATAGCATACTCG |
| 1533 | TCCCATAGATAGCTCGCTGG |
| 1534 | TTCGCATGAGTGCTGAGTAC |
| 1535 | TTCCATATACTGGTCGGCAG |
| 1536 | TTTATGATATGCGTCGCGGA |
| 1537 | TTTCTTATATGCGCGAGCGG |
| 1538 | TGTTGCATATTAGCGGCTCG |
| 1539 | TATATGACATCTCTTGCCCG |
| 1540 | TTGTCACATTTGCGCTCCGA |
| 1541 | GCATCCGAATTGCGACGACT |
| 1542 | GGATCTGAATTGCGCGACCA |
| 1543 | GGCTATGAATTTCGCATCAC |
| 1544 | GGATATGCAATTTGTAGCCC |
| 1545 | CAGCGTATAGCAAGATGGAT |
| 1546 | CGAGCGATAATCAAGTCGAG |
| 1547 | CGCGGATGACACATACTCAG |
| 1548 | CGACGAGCACCAATTCGAGA |
| 1549 | CCGTAGTGACCAATGCAGAC |
| 1550 | GCGATATACATCATTCGGAC |
| 1551 | GACAGTCTAATCACTCGTAC |
| 1552 | GCAGTTATACTAAGGTGTGC |
| 1553 | GCAGTAGTAATGAGTGTCAC |
| 1554 | GCAATGTAGTCGAAGTGTCT |
| 1555 | GCATATAGATACCATTCGCG |
| 1556 | CGAATACTAGACACATTGCG |
| 1557 | CAACTACAGTACACAGCGTG |
| 1558 | AGACACAGAACTACCGCGTG |
| 1559 | ATAGCACAACGTAGACGCCG |
| 1560 | ATACAGTCAACTACATCGCG |
| 1561 | AGTACAACCTAGAATCCGGC |
| 1562 | GAAGACTACTAGATACGCGC |
| 1563 | CGATAATACTACAGACTCCG |
| 1564 | CCGTGCGTACACATAGATCA |
| 1565 | CGTGAGCGACACATGATCCT |
| 1566 | CTGTAGTGACATATAGAGCG |
| 1567 | ATGTCGTCACACAGAATACG |
| 1568 | ATGCTACGAACTACCAATCG |
| 1569 | ATGATAACGTACACACCTGC |
| 1570 | TCGGTCTACGTCTGCTCAGT |
| 1571 | GGCTCACGATCCACTGGTTA |
| 1572 | TGCCTGATACCTTGGATGAC |
| 1573 | GGCCGTGAATTATCATAGAC |
| 1574 | GGCTTGGACGCATTGATAAC |
| 1575 | CCCATCGAAGCATGTGTAAA |
| 1576 | CGGCATCGAAGGCGTTCATA |
| 1577 | GCCAGTTGACCACTTCTGAG |
| 1578 | TCGCATTAGCCATGTGGAGC |
| 1579 | GCAATCTAGTCTAATGGCGC |
| 1580 | CTAAGATGTTCTAATCGCCC |
| 1581 | CCAATAGTAAGTAATGGGCC |
| 1582 | TCATTATACTCTGATGGCCC |
| 1583 | ATGCTAATAACTGATCGCCC |
| 1584 | AGTGTCAACCATGATGAACC |
| 1585 | AGAGCATAACATCATGGCCC |
| 1586 | AGAATCTAACAGCGATGCCG |
| 1587 | ATTTAGACAAGTCGATGGCC |
| 1588 | ATATTAAGAAGTAGGCGGCC |
| 1589 | CATATCAGAATACGATGGCC |
| 1590 | GATATACAGGATTATGGCGC |
| 1591 | CATAAATTGGTTCAGACCGC |
| 1592 | GAAACTCCAATTCAGCGGAC |
| 1593 | GAACAATGAATTTAGCGGCC |
| 1594 | TTCCATTAGATGTGATGCCC |
| 1595 | TATCATATCATCTGAGGCCC |
| 1596 | ATCAGAAGAACTGCACGTCC |
| 1597 | AGCACAAGAACTACGCGCTG |
| 1598 | AGCAAAGAACCATGCCGCGT |
| 1599 | TAAAGAGCAATGTGGCGTAC |
| 1600 | TTCAGGGCATTGAGCGTAAA |
| 1601 | TTAATGGGCTTGAGCGTATC |
| 1602 | TTAATGCGGTTGAGATCGAC |
| 1603 | GCAGGGATAGCAGATACATC |
| 1604 | TCAGGAGAGGCATCGCATCA |
| 1605 | TTATCTTAGGGATGCGGATG |
| 1606 | TGTGCTCTAGGTCATCCGAG |
| 1607 | TTGTATCTAGTGCGAGGCAA |
| 1608 | TATTATCTAGTATGCGCGGC |
| 1609 | TAGTTATCAGAGTGACTGCG |
| 1610 | GTTAGATCATAGTCACCGCG |
| 1611 | GTTAGTATAGATTGGCCGAC |
| 1612 | GTGTTTATACGTTGAGCACG |
| 1613 | TTATCTGTAGTCATCGAGGC |
| 1614 | TGATACTGAGTTAGCGAGCT |
| 1615 | GTGATCTCAGAGCGCAGCTT |
| 1616 | CAGATGTCAAGACGCGGAGT |
| 1617 | CTGGTCAGACAGCGGAATCT |
| 1618 | CGTGGCAGACAGCTAGATAT |
| 1619 | GTGCCGAGACTCCACTGTTA |
| 1620 | GCGGACAGCTCTCCTAGTAT |
| 1621 | ATGCACAACTATCAAGCCTG |
| 1622 | GTGCTTTACTAGCGGAGCCA |
| 1623 | TAAATATCGTATAGGCGGCG |
| 1624 | TAATTCTACTATACGCGGGC |
| 1625 | TAAATCGTATGTAGCAGCGC |
| 1626 | TCCTTCACTGTAGGCTAGGC |
| 1627 | TCAGTTATATGAGCCGACTC |
| 1628 | TCACGTATATTGACTCCGAC |
| 1629 | TCACCGTATTCGAGGCGACA |
| 1630 | TCGTACTGATTGACGGTGAT |
| 1631 | TCACAGCGGTCGAGGTTACT |
| 1632 | TTCACGCGGTCGCAGTATCT |
| 1633 | TACTTGACGTGACTGCATCG |
| 1634 | CGTCACAGAGGACAGCATAC |
| 1635 | TCACTAGAGCGTCGAGCTGT |
| 1636 | TCTACAGTGTGTCAGAGTGA |
| 1637 | CTACCTAATCGACAGCAGAG |
| 1638 | CACCGATAACTACAGCAGGG |
| 1639 | CAACGTCTAGGACAAGGCAG |
| 1640 | CACTAGCTCAGACAGACGAG |
| 1641 | GACTTTACAGTACGATCAGC |
| 1642 | GACACTGACTGACATCGAGA |
| 1643 | GAGACAGTCGAGCGATCAAT |
| 1644 | GCACTTGTACGTCCAGTCAG |
| 1645 | GTACACGGACTGCCAGCATA |
| 1646 | GTAATACGCTATCAGCAGAC |
| 1647 | CTAGATAGACATCACTCACG |
| 1648 | TAGACTCTCGATCAGCCGTA |
| 1649 | GACTTGCACGTACAGCCGAA |
| 1650 | CTTATGCGACACTAGCTCGA |
| 1651 | CTGATGCTACACTAGGCACA |
| 1652 | GCAGACGCACTATCATATAC |
| 1653 | GCAGTAGACACTTCTCACGA |
| 1654 | GCAGGTACACTGACCGACTA |
| 1655 | GCACATCACTGCACGATAGA |
| 1656 | GCAATGACTTCGACTCCAGA |
| 1657 | GACAAGTCATTTACAGGCGA |
| 1658 | GTAACTTGTTTGACAGTGCG |
| 1659 | GACACTGCATGGACAGCGTA |
| 1660 | GCAAGGACTGAGACATGCTT |
| 1661 | TGCGAGGTAGGTTATATCTC |
| 1662 | TGCGGAGAGTGATATACTTC |
| 1663 | GGCGTGAGAGCATTATATCT |
| 1664 | GTGCTGCGAGAGTATTATCT |
| 1665 | CCGCGTGTACCATATAATAC |
| 1666 | GAGCGTGGACGATATACACT |
| 1667 | GGCCGTGTACGATTATGACT |
| 1668 | GTAGCTTGACGATGCTGACT |
| 1669 | GTGCTGGTACTAGCTGCTCT |
| 1670 | TAATGTGACGTAGCCGACTC |
| 1671 | TACCGAGTGCGAGATGCTCA |
| 1672 | TACCGATGTCGATAGATCCA |
| 1673 | TCTCGTATAGGATGAGCAAC |
| 1674 | TCGTGAGTAGGATGCTTTCA |
| 1675 | TACGTGAGATGATGATCGCT |
| 1676 | TAGTCGGTAGCATGAGTCTA |
| 1677 | TAGTTCGAGGAGTAGTCATC |
| 1678 | TAGGTACAGTGCTGGATACT |
| 1679 | CTGCGTCAAGTGTGTAGAAT |
| 1680 | TGTGCGCTAGAGTCTGTCCT |
| 1681 | GGTGCGTCACGATCTCCTAT |
| 1682 | GTGTGGGTACTATGCCATCA |
| 1683 | GCTGATGTACTATCCATACC |
| 1684 | GCTAGATGACGATCAGGTAC |
| 1685 | GCATCTGTACGATCTCAGCA |
| 1686 | GCATCACGACGATTATCAGA |
| 1687 | GCTACGTTACCATGTGCAGA |
| 1688 | GCGTAGTTACCATGCTCACA |
| 1689 | GCGTGAGCACACTCTATCAG |
| 1690 | GCGTGCGAATTATGTATCAG |
| 1691 | TGTGGACACTTCTTATAGGC |
| 1692 | GCGTGAGTAATTTGACTACG |
| 1693 | AGGTGCGTACAAATGCTATG |
| 1694 | CGCAGCCGAAGTACGCTATA |
| 1695 | CGACTGCTAAGGAGCGTACA |
| 1696 | CGATGTTGACAGACCGCACT |
| 1697 | CATGTAGAACTGACTCACAC |
| 1698 | CGAGCGGTAAGGATCTCACA |
| 1699 | ACACGCTGAAAGAGTACGCC |
| 1700 | GATCTGACAGGTAGCGATAC |
| 1701 | TCTCGTGCAGGTAGCTGTCA |
| 1702 | GCTCGGACAGATCGGTATCA |
| 1703 | GCCGGTATAGCTCGATATGC |
| 1704 | GCTGATACAGTTCGATAGAC |
| 1705 | CCTGACTAAGCTCGATAGAG |
| 1706 | GCTGATTACGATCTAGTAGC |
| 1707 | GAATGCTCACGACGAGTAGC |
| 1708 | GAACTGTCCTGACGAATGAG |
| 1709 | TTACTGTCTATGCGATCCGA |
| 1710 | GTTATGTCATCGCAGATTCC |
| 1711 | AGCTATATCAAGCAAGCGTC |
| 1712 | GCTTATACAGTGCAGTAGAG |
| 1713 | TTAAGTAGGTAGCTGGCCTC |
| 1714 | CAAGAGTAACTGCAAGGCCC |
| 1715 | CACTAAGACATGCACAGCGG |
| 1716 | CCTAGTGCAGACCACATGAT |
| 1717 | TCATGCACGTCGCCATAGGT |
| 1718 | TCTATACGCTCGTGCAAGGA |
| 1719 | TCAAGCCCGAGCCGAGTTTA |
| 1720 | TCAGCGCCAGCATTCATGGT |
| 1721 | CCATGCGGACCAAGTCGATA |
| 1722 | GAATGCCGAGCAATGATCCT |
| 1723 | GAATCGGCAGCAATACTGTC |
| 1724 | GAAGCCCAGCTAAGTGGTAT |
| 1725 | AACAGCCCAAACCGGATGGT |
| 1726 | TAAGCACCTTGCAGGATAGA |
| 1727 | TCAGCCCGATCCAGGGTATT |
| 1728 | TATGCGCCCAGGAGGCTTTA |
| 1729 | TGCCCAGCAGGTCGGATTAT |
| 1730 | TAGCTCGCATCACTGACGGA |
| 1731 | GGTCCCATACGAGTGGCATA |
| 1732 | ACTAACCCAACAGCGGAGGT |
| 1733 | CAGCTCTAAGCAGCAGAGGA |
| 1734 | CAGGTCAAGCACATACCAGT |
| 1735 | CTGTGCAATCACGCCAGAGA |
| 1736 | CGGCGCAATAATGTCACAGA |
| 1737 | CGGGACATAATTGACACAGT |
| 1738 | AGGGCCAGACAATACACCGT |
| 1739 | GAGGTCACAATTTGCTACAC |
| 1740 | CAGGCACAAGATTGAGCACG |
| 1741 | ACAAGCGCAAATACTGCCGG |
| 1742 | ACAATCTGAAATAGCGCGGC |
| 1743 | ATCGACCCAAGAATAGCTCG |
| 1744 | ATAAGCACAAGCAGCGCGGT |
| 1745 | AACACTCCAAACCGAGGGTG |
| 1746 | AATCTATCAAAGCGACGGCC |
| 1747 | ATTCCCATAACGCGGAGGAC |
| 1748 | ATGCCAGCAACGCGCTAGAA |
| 1749 | ATGCTCACAAGCCACGAGAG |
| 1750 | ATGCTCCAACGATACATACG |
| 1751 | CAGCTTCAAGAGTACATACG |
| 1752 | CATGTCACAAGGGCATAGAC |
| 1753 | CATGGTCTAAGCCCTACAGA |
| 1754 | ACATGGCGAAAGCACCACGT |
| 1755 | CTTAGTTCAATGCACGCACG |
| 1756 | CGCCAGTTAATGCACGACAG |
| 1757 | CAGCAGCAACTCGACTAGAG |
| 1758 | CCGAAGTCAACTGCGCTAGA |
| 1759 | CCAGTGTCAATAAGAGACGT |
| 1760 | CCAGGCGAACTGATCGTAAA |
| 1761 | CCTGGTACAATCAGTAGCAA |
| 1762 | CTAGTGGCAATCATCAGACA |
| 1763 | CAATGCGAACTCACTAGACG |
| 1764 | CATGGCGTACCAATACCTAG |
| 1765 | AAGTGGCCCAAATAACTGCC |
| 1766 | CAAGGCCCAATACACAGGGT |
| 1767 | GATCTGCCAATGCCGCGATA |
| 1768 | GATTCGCCAATGTGCGCTAA |
| 1769 | GAGCCGCCAATGTCACTAGA |
| 1770 | GCGCCCGGAATGTCGTATAT |
| 1771 | GCCGCGCCAATGTTACGTTA |
| 1772 | CTTCGCCCAATGCGTAGGAA |
| 1773 | TTCCCATGATCGCTGACGAG |
| 1774 | TTGCGGGAGCTGCCTCTTAA |
| 1775 | TTTCCCGGATAGCCGCTGTA |
| 1776 | TTTGCTGGAGTATGCGCTCA |
| 1777 | TTGTTCTCAGCTTGCGGCAG |
| 1778 | TGTGTGGCAGCTTAGTTCAC |
| 1779 | TCTTGGGTAGCATCTGTCAC |
| 1780 | TGGGTGTCAGCATCTACGCA |
| 1781 | TTGTGGCAGGTATGCTCCAA |
| 1782 | GTTGGGCACGGATCTCTATA |
| 1783 | GCCGAGGCACCATGCTTATA |
| 1784 | CGCTTGGGACAATCGCGTAT |
| 1785 | CCGCAGGGAACTTCAGCATA |
| 1786 | TGGAGGGCAGTCTCTCATAA |
| 1787 | CTGGGTGCAAGTTGTATCAA |
| 1788 | TGGCGCACATGGTGTCATAA |
| 1789 | TGGCATCACTGCTGCGGAAT |
| 1790 | TGCCAGTCATCCTAGCGTGT |
| 1791 | TCAGGCCAGGACTGCTTATC |
| 1792 | TTGGCATAGGAGTGCTTCTA |
| 1793 | TTTGCAGACGGTGTGCTATA |
| 1794 | TTGAGTCAGGGTGCCCAACT |
| 1795 | TTTAATATCGTTGCCCGAGC |
| 1796 | TCAGGATGATGAGCATGTAC |
| 1797 | CTCAAGCTGGGAGAACAGTA |
| 1798 | TCAGAAGTGGCTGGATCATA |
| 1799 | TCTCACATGGCTGGAGCATT |
| 1800 | CTACTGACACTGACCAGGGA |
| 1801 | TCGTAGCGACTCTCCAGGTT |
| 1802 | TACGTGTCACTATCGTCGAG |
| 1803 | TATAGTTACGTCTCGCACGC |
| 1804 | TACCGTTACGTCGCTCAGAG |
| 1805 | CACTACAACGTGCTACAGAG |
| 1806 | ATAGGTATAACGCAGTACGC |
| 1807 | ATAGCAGTAACGCATAGTCC |
| 1808 | ATAATCGTAACGCACCGACG |
| 1809 | ATGAGTGTAACGCCTCGACA |
| 1810 | ATGTAGCGAACGTACTCACA |
| 1811 | ATCTAGCGAACGGAACTATC |
| 1812 | GTAGAGTCACGATGCAGTAC |
| 1813 | GTAGTATGACGTAGCAGTAC |
| 1814 | GTACGTCGAGCTAGATCGCT |
| 1815 | GAGTCTGTACGAGGTATCAT |
| 1816 | CGTGTCTTACAGCACTACAT |
| 1817 | CGTGCGCTACAGCAGTCATT |
| 1818 | GTAGCCTAGACGCAGTCGTA |
| 1819 | CGTCTCGCAAGTCGCGTATA |
| 1820 | AGTCGCGCACAGCAACGTAT |
| 1821 | ATCGAGGTAACGCCATATAC |
| 1822 | CTCGTGACATAGCCATAGAT |
| 1823 | ATGCGACGAACGCGGATATA |
| 1824 | CTAGACAGACTGCGACATAC |
| 1825 | TAGTCGTAGAGGCGCTATCA |
| 1826 | CTATCGAAGTCGCGTGAAAC |
| 1827 | CTGCGTATAGAGATCAATCC |
| 1828 | CCGCGTATAGACAGATATGA |
| 1829 | CTCGGTTACGACAGACTGGA |
| 1830 | CGCGCAGGAGACATAGCTTA |
| 1831 | AGCGTCACACACAAGACTGG |
| 1832 | CCTACGAGACACATGACAGG |
| 1833 | CGCCGAGTACACATGCAGAT |
| 1834 | CCGTCGATACAGACTCAGAT |
| 1835 | CTCGTCAGACAGAGCGGATT |
| 1836 | GTCTCGCCACGTATCGGATT |
| 1837 | TCTCGCGTACTTAGGCATCA |
| 1838 | GTCTCGGTACGATGTAGCAA |
| 1839 | CGTGTGAGACAGTAGCATAT |
| 1840 | CGTGTAGCACAGCGACGATT |
| 1841 | GTGTAGCTCAGTCAGCATCA |
| 1842 | AGGTAGATAACGCTAGATCC |
| 1843 | CTGTAGAGACATCTGAATCC |
| 1844 | CTGATACGAAGTCTTATGCC |
| 1845 | CACGCTCGAAGACTAATGAC |
| 1846 | CACGCGATAAGACGTATAGC |
| 1847 | CTAGCAGTAAGTCTATGCAC |
| 1848 | CGTAGTTGAAGTCATCGACA |
| 1849 | CGCGATAGAAGTCAGGACAT |
| 1850 | GACGGACGACATCTGAGCAT |
| 1851 | CATAGACGAATACAGCGGGC |
| 1852 | GATCACGACCTACTAGCAGG |
| 1853 | AGATATAACGAACTCTCGCG |
| 1854 | GATTATAGACTACTGAGGCC |
| 1855 | GAGTTTATACTACAGTGCCG |
| 1856 | GTCACTTACGCTCAGGCAGA |
| 1857 | TCGCTAGACGCTCTGGCATA |
| 1858 | GTACGCTCAGCACTGGCATT |
| 1859 | GACGCGCTAATACTGTCACA |
| 1860 | GCGTGCATACGACTGCCATA |
| 1861 | TGTAGTCTAGTGCATGGTCA |
| 1862 | GTATAGTCAGAGCTGGCACC |
| 1863 | CGTCAGTCAAGTATGGCACA |
| 1864 | ACGAGAGTAAATATGCTGCC |
| 1865 | ATAGAGCGAACGATAGTTGC |
| 1866 | ATCTGACTAACGATGATGCC |
| 1867 | GTTGTAGGACGTATGATCTC |
| 1868 | TTAGTCGAGTCTATGAGCCC |
| 1869 | CGACGATACAGTAATCTAGC |
| 1870 | CTGATACAGGCATAGACATC |
| 1871 | GGTATCAGAGCTAGGACTAT |
| 1872 | TCTATCTCAGCTACGGTCGA |
| 1873 | TCAGTTCGATCTACGGCTAG |
| 1874 | TCAGTGCGACTCAGGTACGA |
| 1875 | GTCACTGCACTCACGGTAGA |
| 1876 | TAACGAGTCTTCAGCACGTA |
| 1877 | GAAGTCGCCTACATAGCCTA |
| 1878 | GAAGTCCGTTACATGACCAT |
| 1879 | GTCAGAGGATCGAGCCACTT |
| 1880 | GCGAGACAGGTCAGTACAAT |
| 1881 | CGTCAGAAGGCTCGCACATA |
| 1882 | GCATACAGGTTACGACGCCT |
| 1883 | GCGATACAGGTTCAGAGATA |
| 1884 | GGACGCATAGCTCGCAGTAT |
| 1885 | GGACGCAGATCGCAGCATAT |
| 1886 | CGGCGTTAATCGCAGAGAAC |
| 1887 | CGCGTTCTAAGGCACGGATA |
| 1888 | CGCGTCGCAAGGCTGTTATA |
| 1889 | CGATACGCAAGGCTACGACA |
| 1890 | CATCTAAGGACACTACACTG |
| 1891 | TATCATCGAGGACTCAGTGC |
| 1892 | CACCGAGCAAGACTGACATG |
| 1893 | CGCACCCGAAGTCAGAGATA |
| 1894 | CGGCTAGGAAGTCAGCATAA |
| 1895 | ATGCTGCGAACGCGCCATAA |
| 1896 | CCGCGTGCAACGTGTTCATA |
| 1897 | GTCGCTGCATAGCATCTCAG |
| 1898 | GTCTGTGCATAGAGCGTCAT |
| 1899 | GTGGTGTCACTGATACGTCA |
| 1900 | GGTTAGCACTAGATCGCACT |
| 1901 | CGGGATCTACAGCATCATAG |
| 1902 | CTGGATATACAGCACTCACA |
| 1903 | ATGCGGCTAACGCCTCATAA |
| 1904 | TCGCGGCGCACTCTGTTATA |
| 1905 | TCGTGCTACTGCCACTGTAT |
| 1906 | TAGGACACTTCGCCACTATG |
| 1907 | TATGACAGTTCGCGCTACCG |
| 1908 | TCGCGCAGTTAGCCCTATGT |
| 1909 | TAGCCACCGTAGCTGATCGT |
| 1910 | GTAACCCGCTATCAGATCGA |
| 1911 | AGAGCGCAACACCACATTGT |
| 1912 | AGGCTAAGAACGCACACTCG |
| 1913 | GAGCCTAGACAGCTTCATAC |
| 1914 | GGCAGTTCACGACTCGACAT |
| 1915 | GGCCTTAGACGACTCGCATA |
| 1916 | GGTCGATCAGCACTGCATAC |
| 1917 | GGAGAGTCAGCACAGTCCTA |
| 1918 | GTATAGGCAGCACGGCTCAT |
| 1919 | GCACGGCGAGCACTATCTTA |
| 1920 | TAACGTCCTGCACGATCTGT |
| 1921 | GGACGCCTAGCACATCTGAT |
| 1922 | CGCTGCACATCACATGGATT |
| 1923 | GCACATCGAGCACATGCAGT |
| 1924 | GCACGACCAGCTCTTAGGAT |
| 1925 | CCCACCAGACAGATAGAGGT |
| 1926 | CCCGACGCACGAATAGATAG |
| 1927 | CCCACGACAGATACATGAGT |
| 1928 | CTTCGCGCAGCTACATAGAT |
| 1929 | CGCTCCGAAGCTGCGATAAT |
| 1930 | CGCCGCGTAAGCAACAAATT |
| 1931 | CGACGCTCAAGGACTCATAA |
| 1932 | CGCACACTAAGGATCATTAC |
| 1933 | AGACACGCAAGAAGCTGGCT |
| 1934 | GCACGCATAGCAGAGGATCT |
| 1935 | GCTACGTCACTGAGCAGGAT |
| 1936 | GTACATCTCGTGAGCAGAGC |
| 1937 | CTACACGACTTGAGACGAAG |
| 1938 | CTAAGTACGTGCAAGCAAGG |
| 1939 | GACACGTAGGACAGCTATGC |
| 1940 | GACATAGTAGACATCTCACG |
| 1941 | GACAGCGTAGACATCGTCAG |
| 1942 | GACTATCACGACATTCAGCG |
| 1943 | GATCTACACGCTACCAGTGG |
| 1944 | GCTTACTACGGATAGATCAG |
| 1945 | GCGTATCTAATGGAGTAGCA |
| 1946 | GCGTATTTACAGTGAGCGAC |
| 1947 | GCGTATATCGAATTGAGTGC |
| 1948 | GCGTTCACAGAGTCCACGAT |
| 1949 | CGCGTATCAAGGTCACGACA |
| 1950 | GCTATTACAGTGTCAGAGAC |
| 1951 | CGTCAGATAAGGTGAGTTAC |
| 1952 | CGTCTGTGAAGGTCAGCTAA |
| 1953 | TATTAGCACTCGTCAGCAGC |
| 1954 | ATGTTATCAACGTCAGCGAC |
| 1955 | GGCATACTAGAGTCAGCGAT |
| 1956 | AGTGCGATACAATACGAGCG |
| 1957 | CAGCACACAGAGTACAGCGT |
| 1958 | CGTAGCATAAGGTCAGCACC |
| 1959 | GTCCATAGACGTTGATACCA |
| 1960 | GCTACGATAGATGAGCCACG |
| 1961 | CGGAGTACACCAGATCCAGA |
| 1962 | GAGCGTATAGGAGATCCAAC |
| 1963 | GACTGTAGAGAGACGATCCA |
| 1964 | CTAGTAGGAAGTGCGATCAA |
| 1965 | CGTAGAGGAAGTGATACTCA |
| 1966 | CGTATCGGAAGTGAGTATCA |
| 1967 | CTATGACGAAGTGAGAGTAC |
| 1968 | GTTCGTAGAGATGATCGTCA |
| 1969 | GTTCTCAGATAGTATGCAGC |
| 1970 | AGTCTGTTAAGATATGCGCC |
| 1971 | AGCACGGAACAGTAAGCCCT |
| 1972 | ATCCAGAGAACGTGAGATCC |
| 1973 | GACAGTGTAATATGAGGACC |
| 1974 | CATAGTAGAAGATTCGAGCC |
| 1975 | TGAGATATAGTATGCGGCCA |
| 1976 | ATGAACATACTATACCGCGC |
| 1977 | TTCTCTATATCGTGCGCGGA |
| 1978 | TGAGTTTACGTGTATGGCAC |
| 1979 | ACGGCATCAAAGTTGCATAC |
| 1980 | ACGGGCTCAAAGTATGATAG |
| 1981 | AGGCGCTTAAATGTGGATAC |
| 1982 | CTGCCGTTAATGGCGGACAT |
| 1983 | CTGAGCCAATAGGCGCACTT |
| 1984 | TAGGCATGATGAGAGCTATC |
| 1985 | TGCCTATGAGGAGTATGAAC |
| 1986 | GGGCTATAATGAGCTTGACT |
| 1987 | TAGGCTTCATCAGCTATCAG |
| 1988 | ATTGCTTCAACGGGCATTAC |
| 1989 | TATGATCCATGCGACTCGGA |
| 1990 | TTGTATCCATCGGCCCAGTG |
| 1991 | ATCAAGGCAACCGCCAGTAG |
| 1992 | TCTCAGCCATCCGTGATAGG |
| 1993 | TATCAGGCATCCGAGCATAG |
| 1994 | TTAAGCTCCTCAGTCCATGT |
| 1995 | TAAGGGCGATGAGCCTATCT |
| 1996 | TAAGGCCGAGGAGCTTTCAT |
| 1997 | TAAGGCAGTGGAGCCCTCTA |
| 1998 | TGGACAGGCTGCGCTCTATA |
| 1999 | CTGGAAGCCTGCGACCAAAT |
| 2000 | TCAATGCACTGAGCCCGAGA |
| 2001 | GATTCACACTGACCCATGTA |
| 2002 | TAAATAGATTGGAGACGCGC |
| 2003 | GCATTAGAAGGTCTGGACTA |
| 2004 | ATTGGCATAACGTATTGCGC |
| 2005 | CAGGACTGAAGATCGAGTAC |
| 2006 | TAGAGTCAGTCATAGCTCGA |
| 2007 | TTTATCGTAGCTGGCTGCCC |
| 2008 | AGGATTAGAACCTACGCACC |
| 2009 | GCCGTGAGACCACTGTACTA |
| 2010 | GACGCTGAATCCTATTGACA |
| 2011 | CGCCTAAGGATCGTGAAGTA |
| 2012 | CGACGACGAAGCTGCATGAA |
| 2013 | ACTCGAATAACAGCATCTCG |
| 2014 | CCCGTAAGCATGGCACAGAT |
| 2015 | CAATACAAGATTACGGCCTC |
| 2016 | GATCAGAATCTATGGTACGC |
| 2017 | TCTGTGTACTGCTCGCCAAT |
| 2018 | ATATTTGGAACGCAGCTCAC |
| 2019 | TGCAGTATCGCAGCGGTTCTA |
| 2020 | GGGCAATGTTTATCCACAGA |
| 2021 | CTGACCGAATCCAGCAGAGA |
| 2022 | GATCGTGAATCCGCGCACTA |
| 2023 | GAGCCGTAATCCGAGCGATA |
| 2024 | TACTCCTGACGACTTAGGCA |
| 2025 | TGCTGTCACTCGGCGTCTAT |
| 2026 | GTACTAGCATATCATCGACG |
| 2027 | TATCGCATAGATCAGTGAGC |
| 2028 | TACGGGCAGCCAGGTACTTT |
| 2029 | GTTCATCACGAGTGCGTAGA |
| 2030 | CATGTATCAAGATGGCTGAC |
| 2031 | GGTCGCGCATTCCAGCATA |
| 2032 | GCACATATCTAGCGACATCT |
| 2033 | ACGCGGCTAAAGGTAGATAC |
| 2034 | CACTGCCCACAAGATGTAGA |
| 2035 | GGATTTACATGGCCTAGCAA |
| 2036 | CATGACACAGAATCGACCGT |
| 2037 | AGAGGCATAAATGAGTCTCC |
| 2038 | TGAGTAGTACGTTACGCCTG |
| 2039 | CGATAGCGAAGGAGTCCACA |
| 2040 | ACACTCTGAAAGACGCGACG |
| 2041 | GTCTTAATGTTGGGCAACG |
| 2042 | GTTATCGACTACGCTGTACT |
| 2043 | TCGTGAGACCGTCGTCAGTA |
| 2044 | GACAGCGCAGTACAGGTAAT |
| 2045 | CGTACAGTAAGTATGATGCC |
| 2046 | TAGAGCATCTGACGCTATGA |
| 2047 | GTCACGATTAGTAGGCACG |
| 2048 | TCGTACCTGTATTCAGCGCG |
| 2049 | TTAATCCGCTGTAGCCCAAA |
| 2050 | TTAATTGACTTCGCTCCAGC |

In accordance with one aspect of the present invention, Tag genes were made by annealing and extending overlapping 23 to 192 oligonucleotides randomly chosen from the 20mer Tags or their complements from Seq. Id. Nos. 1-2050 asembled head to tail.

In accordance with the present invention, Tag genes preferably comprise 5 to 1000 randomly chosen 20mer Tags sequences from Seq. Id. Nos. 1-2050 or their complements. More preferably, Tag genes comprise 10 to 500 randomly chosen 20mer Tag sequences or their complements. Still more preferably, Tag genes comprise 20 to 200 randomly chosen 20mer Tags sequences or their complements.

In accordance with one aspect of the present invention, a Tag gene is incorporated into a vector having a first promoter sequences 5′ to the Tag gene and a poly(A) tract 3′ to the Tag gene such that a sense polyA⁺ RNA is generated from transcription initiated from the first promoter; a second promoter sequence is located 3′ to the Tag gene and on the opposite strand as the first promoter such that antisense RNA can be synthesized from the second promoter of the Tag gene. The choice of synthesizing sense or anti-sense Tag gene sequence will depend on the ability of the transcript to bind to Tag probes place on the nucleic acid array. In accordance with one aspect of the present invention, one or more endonuclease restriction sites may also be incorporated into the Tag gene contructs.

Preferably, in accordance with one aspect of the present invention, the first promoter is a T3 promoter. In a preferred embodiment the second promoter is a T7 promoter. Transcription can be performed either in vivo or in vitro, in accordance with the present invention. It is also preferred that the nucleic acid array is an Affymetrix GeneChip® Array.

In accordance with one aspect of the present invention, sense RNA containing the Tag gene sequences and the poly A tail synthesized from the first promoter can be spiked into samples, containing for example mRNA, and subsequently hybridized (after labeling) to a nucleic acid array having appropriate Tag probes (i.e. probe sequences complementary to the Tag gene in question). With a nucleic acid array having the appropriate Tag probes, spiking can serve as a control for various aspects of the assay process such as variations in sample preparation, hybridization conditions, and array quality. In accordance with one aspect of the present invention, anti-sense transcripts of the Tag genes can also be used as control spikes for a nucleic acid array having appropriate probes.

In accordance with another aspect of the present invention, the synthetic Tag gene DNA itself can also serve as spikes in applications involving genomics. For example, Tag gene DNA could serve as a control for PCR, including long range PCR, fragment labeling, sample preparation and as quality control for the nucleic acid array.

The invention will be further illustrated, without limitation, by the following examples.

### EXAMPLES

### Example 1

Construction of Cloned Synthetic Tag Genes

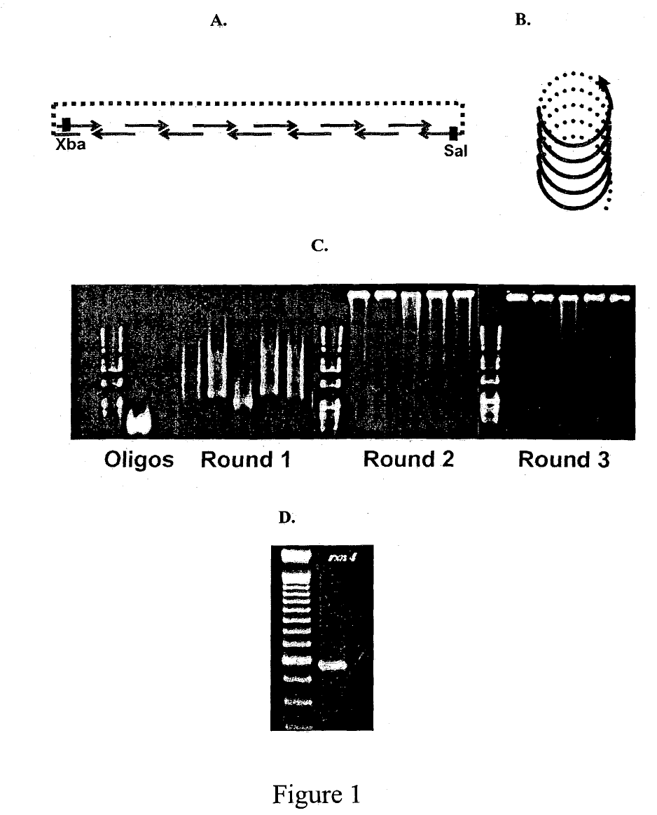

In one embodiment, thirteen different Tag sequences of varying sizes were designed by randomly assigning 20mer GenFlex™ Tag sequences chosen from Seq. Id. Nos. 1-2050, set forth above, to groups, and orienting the sequences head to tail. 60mer oligonucleotides were designed to encode the desired genes as well as flanking sequence used for assembling and cloning the genes. The gene assembly with unpurified 60mers can be accomplished by polymerase extension of the annealed oligonucleotides as depicted in FIG. 1 and described in U.S. Pat. Nos. 5,834,252, 5,928,905, and 6,368,861 and in Stemmer et al. (1995) Gene 164:49, each of which is incorporated here by reference.

Oligonucleotides, nucleotides, PCR buffer, and thermostable DNA polymerase are combined and subjected to temperature cycling. After about every 30 temperature cycles fresh buffer, nucleotides, and polymerase are added to replenish the reaction. Each oligonucleotide serves as both template and primer, and because of the oligonucleotide design, the extended products continuously grow in a spiral of concatamers that can reach over 50 kb.

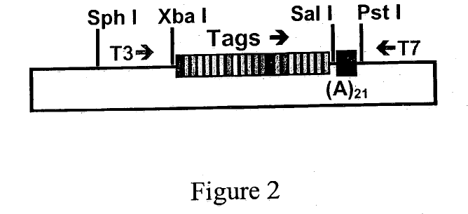

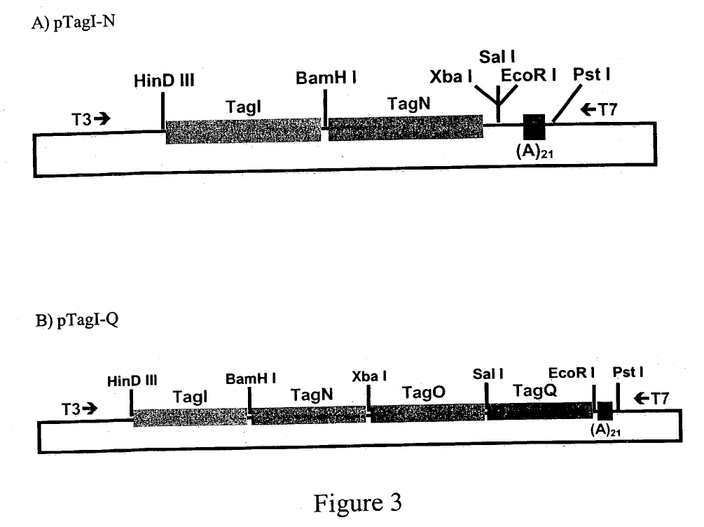

Following assembly of the oligonucleotides into concatamerized products, monomers for cloning are prepared by digestion with restriction enzymes either directly or following amplification by conventional PCR with flanking primers. The digested monomers are ligated to the plasmid vector pSPORT1 (Invitrogen Life Technologies, Carlsbad, Calif.) (see FIG. 2) and the constructions propagated in the E. coli strain DH5α. Subsequently two features useful in generating poly(A) sense RNA are added to each construct: a T3 RNA polymerase promoter upstream of the gene, and a poly(A) tract downstream of the gene. The 13 genes constructed are named TagA, TagB, TagC, TagD, TagE, TagF, TagG, TagH, TagI, TagJ, TagN, TagO, and TagQ. Two additional constructs, called Big Tags, were made: TagI and TagN are combined to make TagIN, and TagI, TagN, TagO, and TagQ are combined to make TagIQ (see FIG. 3). TagIQ is then altered by site-directed mutagenesis to add two restriction sites, EcoRI and XbaI, and the resulting construct is named TagIQ.EX. These additional restriction sites make construct TagIQ.EX useful for as a genotyping assay control (see below). Fluorescent dideoxy DNA sequencing was used to determine the sequences of all the constructs, which are shown below. Organization of a synthetic Tag gene and flanking sequence in the Tag gene clone is shown in Table 1 below. The actual sequences of synthetic Tag genes and flanking sequence in the Tag gene clones are shown in Table 2. The T3 and T7 RNA polymerase promoters and the poly(A) sites are underlined, and the Tag sequence is in CAPS. The DNA sequence shown is the sense (Tag) strand. The length of each Tag sequence is given.

The sizes of the Tag sequences in constructs TagA through TagQ ranged from 467 to 1000 bp, with a total of 9808 bp; the TagIN construct has 1944 bp, and TagIQ has 3849 hp of Tag sequence. There are a total of 78 base pairs different from the designed sequence, a rate of 8 bp per thousand; these changes are fairly evenly distributed and probably arose from polymerase errors made during the assembly and reamplification reactions. There are in addition 3 deletions of 12, 36, and 90 bp, the latter two of which are caused by the introduction of an unexpected restriction site that led to truncation of a gene during cloning. The synthetic Tag sequence in the plasmids does not appear to affect bacterial growth, and the plasmids are stable.

TABLE 1
Organization of a synthetic Tag gene and flanking sequence
SphI recognition site - T3 promoter - spacer - TAG GENE - spacer - (A)21 - PstI
recognition site - spacer - T7 promoter

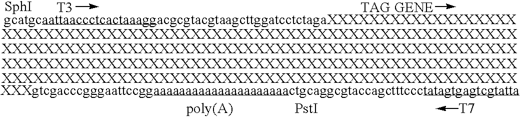

TABLE 2
Determined sequences of the synthetic Tag genes

TagA 501bp
gcatgcaattaaccctcactaaagggacgcgtacgtaagcttggatcctctagaATTTGATCGTAACTCGGGT
GACCAATGACCATATACGGCGTATTAAGGTTGTACCCTCGGTCTCAACTTGTC
GTATGGGACTTTCAAGTACCTTAGCTCGTCGGACGCTTTAGATGACTTATCCA
TAGTCCTAAGTCCGGCGCCGGTTAAGCCGCTATTAGCGTGTGTGGACTCTCTC
TAGGAGCGGCTTCGCACAAATTACTGCTCAATCCTAGATACGTTGCGCTCTTT
GGTAAACGGCTCAGATCTTAGCACTCGTGCAGTTCTACGATGGCAAGTCGTG
CCTCGTTCTCGTGTAGAATATCAGCTAATAGGGTCGGCTCAACAGTGTATCCG
GTGGACAAGCACTGACACGCGATGACGTTCGTCAAGAGTCGCATAATCTCAG
AATCCGTACAGCCGCATCGGGTTCACGGCTATAAAACAGCGTCATCAGCGTA
GGGTATCGCTTCGCGTGTCATGACTTGGGCCACGTCTCTCTCTCGCACATTAG
GCTAGATTgtcgacccgggaattccggaaaaaaaaaaaaaaaaaaaaactgcagcgtaccagctttccctatagtgagt
cgtatta
TagB 467bp
gcatgcaattaaccctcactaaagggacgcgtacgtaagcttggatcctctagaTTTAGTCGTTAGCCCGAGC
TTAACTATTAGCGTCGGTGCTATATCCTTACCGCGTATGGAGTAGCCTTCCCG
AGCATTTGTCTACCTTACCGTCAAGAAAACCATCGACTCACGGGATATTGACC
AAACTGCGGTGCGATTAACTCGACTGCCGCGTGAACAACGATGAGACCGGGC
TAAGGCACGTATCATATCCCTAATTCGCTGAATAGTGCCCTACATATCCTAAT
ACAGGCGCGACGAACCTTATACTCGATGGAAGACAGTTATACCCATGCATAA
AGCTCTATACTCCGAGAACTAGCATCTAAGCACTCGGCTCTAATGTTAAGTGC
TCGACCACAGATCGAAGGTCGGAACTCCAGTGCCAAGTACGATGGCTCACGT
CTTATTTGGGCCGCCAGAGTTATGTTTGAGTCTTCGATGTATGCGCTCGTTGC
CCTATTGTTGTGTCGGATCTTCTAGTTgtcgacccgggaattccggaaaaaaaaaaaaaaaaaaaaac
tgcaggcgtaccagctttccctatagtgagtcgtatta
TagC 579bp
gcatgcaattaaccctcactaaagggacgcgtacgtaagcttggatcctctagaTGTGATAATTTCGACGAGG
CGTTACATATTCTGAGAGGGGTGATTAAGTCTGCTTCGGCCTGGGATGGTCTG
TCTACGTGTGCGTAGTTCTGTCATAGCGTCGAGGATTCTGAACCTGTCCATAG
TATCCTGTAAGCGTCCAATGTACCTATATCGTGGACCCAAAGTCGATACGTCC
GATTAAGCGACGTTGGTCTAGGTAACGAATTATACCCTCGGGTTACGAATTAT
GGCTGTGCCTAACGAATCTGGGACGTGCCTAAGTAATCTGGTCCGCGACTAA
GATGTACGGTGATCGTGGACGCTTGACCGGACTTATGCGTCGCCTTCCGAGTT
ATTGGATGGCGTTCCGTCCTATTGGATACTATTCCGTGCGTGTGCGACACGTT
CCGAGCATATGCTAACAGTTCCGTCACTATGTAACGCTTGACGTAGATTGCTA
TCAGGTTACGATGACTGCTAAGCCATTACGCGACATTCTGCAAAGTTACGTCG
CATTCTCTCACGTTACGGCTGATTCTCTAGGCTTACGCGCATGAGCTCTAGGT
TCCGGGTACTATCGAACGTGTCATTGGTACTgtcgacccgggaattccggaaaaaaaaaaaaaaa
aaaaaactgcaggcgtaccagctttccctatagtgagtcgtatta
TagD 519bp
gcatgcaattaaccctcactaaagggacgcgtacgtaagcttggatcctctagaATAGACTAGCCTGCCGGTC
AATAACTGATGACGCGGAGTCAACCTGATAACCCATAGCGGAACAGTCTAAC
CTACGCGAGATACGTCTTACCGCACATAGGTAACCTATTCGTGACTAGCAGG
CCTTATTCCGGTGCTATGAGTATCTTACCTGGTCTAGGTATCTAATTCGTGAG
TCGGGTACTACATTCGTGCGATGGGTCCTCGCTTCGTCTATGAGGTCTCGTCT
TCGTGAGTGCAATGTATCCGAAGTCGTAGTGATAATATGGAACTAGGCGCGA
TTTGACGAACGTATGCCGCATATTCGGAACGTCGCCTGGAAATTCGCCACCTA
GATCGAAATTATCGGAACTCGTCGCTTATTTACGAACCTTGGGAGCCGTTCCT
AAAGCTGAGTCTGGTTTCTTATTAGCGAGGAGCATTTCGTGAATACTGAGCCG
AATATCGTAAGACATCCGCGAGCGACTGTAAACTAATCGGGGAACTTATTAT
AGAGCCGGTCCAGGTCTTGAACGACGTgtcgacccgggaattccggaaaaaaaaaaaaaaaaaaaa
actgcaggcgtaccagctttccctatagtgagtcgtatta
TagE 578bp
gcatgcaattaaccctcactaaagggacgcgtacgtaagcttggatcctctagaCCATCCGATTAAATACCGT
GGATTACGTTAAGTTACGGCGGTTGACTTAGTTATGCGAGGTTCGCTTACGTT
GCATAGCGGATCGCTTAACCTCTATGCGTACAGCTTACCTACTATGCGTGCAA
GTTACCGAGCTGACGTCGCGTTAGACAGCTCATTCGTCACGTTTAGGACTATG
TCGAAGCGTTTCGACCATGTCGTCTAGCTTAATACCTCTGCGTCTCAGTTAAT
AGTACGGGCAATCCGTTATGTAAAGGGTGACCACGTTTCAGAAGCTGCCATA
TACTTACACAGCAGGCGATCACGTTAGATCCACTGCGTCACGTTACCTACATG
ATCGATCCGATTACAGGCCGATCCATCGGATTACACACGAGTCCTGCACGTT
AGAACACTGGCTCGCGGCTTAGATCAGCTTCCCTCGCTGGAGATCGAATACG
CCCAGCTWAGAGCGAATTGCGGCGCGTTCGACATAATTGCCGACGCTTCGAC
AGAATTGTAGGCGATTCTAGCCAATTGCACGTCGTATTAGGTAGTCACTCTCG
ACCTAGCGTAAGGATCCACGATCCTAGAGTCGGgtcgacccgggaattccggaaaaaaaaaaa
aaaaaaaaaactgcaggcgtaccagctttccctatagtgagtcgtatta
TagF 660bp
gcatgcaattaaccctcactaaagggacgcgtacgtaagcttggatcctctagaACGCGGTCACTCAGCATAT
AGTCGTTGCACCTAGTTGATAGTCGCCGATTCTAGTTATGGCGTCGGATTAGA
CCGGATCACCCGGACATGGACGTTAAGTATCCGGCCTGGACGACAATAATTC
GGCGGTGCCTCACAATATTCCGAGAACTCTGCATCAATTCGGGCTAGTCGTAC
CTGAACGGGCATCAGTCGAATCTCTTCGTGGCTAGTCTGTGACGTCCGTGGTT
CATCGTGTCACCACGCGGTACATGAGTCAAAGTCCGAATAGCTCGCGCAACG
TCCGTCTAGCTGGATCAACCTATCCCTGAGTCTATATGCGTACCAATGGATGC
GGTCTCCTCCGACTGAGTATGCGTTCCTCGGACTGGATCAGCTATCCACGAGC
TGTAATCCGGTACTAGGGTGTATCGCCTGTTACTAGGTTAGACAGTCGTGTAC
TCGGTTAGACTGATGGTCAACGACCTATACTGACAGCATACGAGACGTGACG
ACTGCATAGTGGTCGGTCTGACACATCTCCTCGTTGGTAGTACGTGCCCCGTA
TGGATAGGGCTCTAGCCCGCTATGGTGAGTCTAATCGCCGTTGGTCTGTATGC
AGTGCGGTATGGTTCCTCTCAGTCACGTATGGTTCGCTGCTGTCCGTCATGTG
TTAGATGCgtcgacccgggaattccggaaaaaaaaaaaaaaaaaaaaactgcaggcgtaccagctttccctatagtgag
tcgtatta
TagG 760bp
gcatgcaattaaccctcactaaagggacgcgtacgtaagcttggatcctctagaATGCAGCGTAGGTATCGAC
TCTCACTGTGGAGTCGTCTATGATGTCGTGGAGTCCTCTCAGAGTGCTGTAGG
TCCTCATAGGTCGTGCTGTCTCTCTACACGCGTGCGTGAGTCTACATTTCTGC
GAGTTGGTGCTCTCACTGCGGTGTCAGTGATCTCTCCGCGTGTGACATGAGTC
TAGCTTCGCGGTCATGGTCTATCCCAGCGATGGATGAGACTACTCTGTACTAG
ATGGTCATGCCTGCGAATGAGTCGTCAGTGCCCACAATGTCTCGATAGTGCG
CCGAATGTGTCTGTAATGCCTCGAATGTGTAATCGTCAACTCGTATGTGAAGT
GCTAGGCTAGTATTGACATCTACGGGCGGCTATTGACGAACTCTCCGGTATAT
GCTCTACATCTGCAGGGAATTGCCGACCATATATGGGTCTTGCTGATACGCTA
GGGTGCTTGCTACTTAGATAGGCGTCTTGGCCGCTATTCGCGGCGTGTCTCAG
AATATGCGCGACGTGTCTGGTATATGGCGACTGTGTCCGTCTATACGCATACT
GGTCCACATATAGACATACTTCCACGACATGACAAAGCGTGCTCCTACATAG
CACGAGCGTCTCCTAAATAGATCCGGTCTTATCGCTGAATGTCTAGGATTCTC
GTCAATGATCTACGATCCTCGCTAAGTATTCAGCCACCTCGTATAGTATTCGC
GCACCTGAGGATTTATTCACCTGACTCGCGTATAATATGCCGTCACCTAGTCT
Agtcgacccgggaattccggaaaaaaaaaaaaaaaaaaaaactgcaggcgtaccagctttccctatagtgagtcgtatta
TagH 848bp
gcatgcaattaaccctcactaaagggacgcgtacgtaagcttggatcctctagaGATATGCGTTACGTGAGTC
TGATAGCAGTTCACTACCTGGATATCTGATCCACTAGCTCGATCATGCTCACC
CATAGTTTATCTGCATCACTCGTACTGAAATGCTCACATCGCAGGTAGAGCAG
CATCGTAGAGCGTCAAGCTGCATCCTAGCGTCATGAGTCATAGTACCTCATGC
TCACGTGATCTACCCTAGCTGACCGCTAATGACGGCAGTGCAACCTGAGATA
CCGACGGCATACTGTCGTCAACGTCAGGCAATGTGTCCGAACGGCGAGCTAC
GTCGCCTCACGGAGTAATCGCGTCCCTCTAGGTATAGTGCCGTCGGTTCAGGT
CATATGTCGCGGGTTCTGCACATATCACGGACGTATCGCTATCAGACGGACG
CTCTCGGACCTAAACCGTAGCTCTCGGCAAGATCGTCCTCGTCTCGAATATAG
CGCCCTAGTGCTGCAAATGTCACCGCTATCTCGTAAGGGGTCCGTCTGTTGAG
TTAGGCCTCCTCTCGTTGGATGTGAGCTCGGTTGCTTGGATGGTGCAGCTTAC
TTCGCGTACCTGCTGTTTGCATCAGTCCTCTGCATCTATAATCGCGTATCTCTC
TCTAGTAGACCATATAGCCATCTAAGCGCTCGATATTCCACCTAAGTGGCGCC
TATTGAACTAAGTGGCAGCCGAATGGACTATCGCTCCTCGATATGTACGGAT
AGGCCACGGCATGTACGAGCATAAGCCGAACTGCACGAGCATACCCGACACT
GATCTGAGAGTCGCTTAAATCATCTGCGTGTCTTAGAGCTTATCGCCATGTCT
GTCAACTGTACTGTCATCCTGTAACTGTAGCGTATGTGgtcgacccgggaattccggaaaa
aaaaaaaaaaaaaaaaactgcaggcgtaccagctttccctatagtgagtcgtatta
TagI 940bp
gcatgcaattaaccctcactaaagggacgcgtacgtaagcttggatcctctagaGATAAGCGTTCACAGCTCG
GCAATACCTGTGACGAGCTGCTCGCAAGATTTACGCAGTGTGGCTATACTTG
ACAGTGATGGCGCTTACTTCAGATGTATGGGTGATACTTCGCTATATGGGTGG
TCACTTCTCTATGGCGCGTGACAATGTACTATGGAGCGGTCAATGTCAGTACG
GATCGCGTCGATCTAGGTGACTACGCACGCCTCTGGAGTAAATCGARWGCTC
CGTGCGAAATACGCGGTCATCGTGCGAATAACCGAGTCATCGTGAGTAGTAT
GAACGTGTCGTGTTATGCAGCGGTATGTCGTGCTATAATGGCGTCTGTCGTGC
TCATAAGGTTCCTCTGATGTGCTAGACGTGTCCATCGAGCTGCATAGCTATAC
TTCGAGTCACTTGGGATACTTCGATAGCGTTGTGAATAGTGTCGTAGGCTCTC
GGGCACGTTGYTAAACTGTTGCCGCCAATTCAAGATTAGTCCAGCTCGTACTA
TCGAATACACCATCGTCGTATCGAATAATCGCACCTCGTAGGAGTCAGTTGCC
ACTCGTTGATAGTCAACCAAGCTCGTTAGATAGTAGCCCAGATCCTACGAGA
TGAGCTACGTAACTACAGTGATAGCATATAGGGTACGCTAGAATGCCAGGTC
GTAGTCGAATTAGTCAGGTTGGATGTCTACTAGTTGACTTGGAGTATGCCATG
AAGACTCGTCCCTCGATATCAATACTCGTCCGCAGGTGAACACTGTAGTCGGT
GCTAGTGCCCACTTCTCGGTATGTGTCCTCAATTATCGAGTAGGATTCTAATC
AATCGTCGCGGCTCACTAATYGTCTGCGGTGGCTACTAATGGTTACGGTGCCT
GACTAATCGTGTAGGTGTCTAATACATCGTGATACGGGCGATATAATGCTCG
ATACGGCAAATATAGCTCCGTCCGGTgtcgacccgggaattccggaaaaaaaaaaaaaaaaaaaaac
tgcaggcgtaccagctttccctatagtgagtcgtatta
TagJ 960bp
gcatgcaattaaccctcactaaagggacgcgtacgtaagcttggatcctctagaCAATGATAGGCTAGTCTCG
CGCAGTACATGGTAGTTCAGCCAATAGATGCCTAGTACGCTGACGGCATTCA
GAGTACGCTGATCGGCTTATGACGTATGTGACGCAGCTCTTAGCGCAATGTAT
GTGCTGTTATCGAAGCCTATGGCTGAGTATGTAACGCTATGGCGTGCTAGTCG
TCTCATATACGTCTGATGACCTCGTATCATGTTATAGGGCTGCGAACTGTCGA
TGATGGTCACGACTCTGTCGATAGCTGTGTGACTCATTCAGAAGGTGTGCAGC
CTATATGATACGCAGTCGCATCCTATCTTACGTGTCAGTACTATGTGTGAGTG
CTCCGCCCTAGTGCTGATGTATGCCCCATAGTGCTCAGTGGAGTCTCTCTTAG
CATAGTGTCCGCTCATACATTAGATGGACGGCTCATTAGTATCATCGTCGGCT
GATATAGGTCGTGGCTCCCTGTATATCGAGGTGAGTCTATCTGGATCAACGTC
GCACTATGATGTGCAAAGTGTCGTCCATGTATAGACAGTGCGCGTATCATAT
AGGATGCGGCGATCTCATACAGCGTTACGGTCGCTGCGTACTGTATAAGGAT
GCTCTGTGAACTGTCATCGGTCCGATCAATTAGTCTAGTGTGCGTTATTCAGA
TCGAGTGAGTACATGATTCGTCAGTGTGGATCAATTACAGTTAGGCCGCTGA
CACATTAGTAACGTCGGCAAGCACTTAGTCGTGTCGTAAGCCAGTGTGTCGT
GTCTTAGACGACTGTGTGTGATTCTCGAGCGATTTATACATCCGTGACAGCGT
TTATAGTGTGCTGACAGACTGGTTGGTTATCCAATGATCGACCTGGAGTCTAA
TATCTGACCACGCCTTGTAATCGTATGACACGCGCTTGACACGACTGAATCCA
GCTTAAGAGCCCTGCAACGCGATATACAGGCGCTGCTACCGATATgtcgacccggg
aattccggaaaaaaaaaaaaaaaaaaaaactgcaggcgtaccagctttccctatagtgagtcgtatta
TagN 998bp
gcatgcaattaaccctcactaaagggacgcgtacgtaagcttggatcctctagaAGATCGCAGGGTATCGCAT
CGACAGACCTGGTATCGTCGTGACGAACGTGCTACTCGCTTATCGGGCCTGCT
ACATCAGTGGCGATGTTCGTAACCCTTAGCCGATCTTCTTACTTACGAGGCTA
CTATTCGATCAAACTCGCCTATCTGGTAATAACTGCGGTGATCTGGTAGCCAC
TACGTGCGCCTGGTAGCAAATACGGCGAGCTGGTATCACTATCGGCTCAGTG
GTCCGACATAGTGCCCAGTGGTTCGCATAACTGCCGCTGGGTCCAATATAAC
ACGCAGTCGTCAATCATACGAGCCGATGGTCAGCAATAGCGCCTGTGGTGAC
ACTATGCCACCTCTGGTCTAATATAGCGCCCTGTGGTCGTATAATCGAGCGCG
TAATCGTATATYCGACTGTAGGTGCGTAACTCGCGACTAGGTGGCTCTAATCT
GCGTTGGTTGTCGCTCACAGTGTCTGGTGTTCGATACCCGGATCGGGTTCCGT
AATCTTGGCATCGAGGTTTCGTACATGTCACGCGGTCTCGTTCATTCTCGGTG
GTGCTCAGTACATCCAGTGGTGAGTCGCTACATCACACGGTGATCCGGCTAA
ACCTCTGGGCATCCGTATTAAGCGACATTCCTACGACTTATCAGCACGTCCTA
CGGTATAACAAGGCGTGCTACGGTCTAACGACGCTGGTAGCAGTCTATCAGA
TCGCTAGTACGAGTTAGAGATGCTTAGTACGCCTTCGAATCTATGATGCTCGT
GCTCACGCGATGCACTCGGATTATGGCACATGCACTCGCGTAATGACGCTGC
ATCGCTCAGTATGATCCATGAGCGCCGTGAATGACGCATGAGCCTCGTATCG
AGTGCATGAGCTGTCTTTCACATGATACATCGCTCTAAATCATCATGCGACAG
TCTCGACAGCAGCTCAGCATCTATGCATCATGTGCCTCACTAGGACATCATGC
TCGACTCTGAGACACTGATCGAGCATTAAGACgtcgacccgggaattccggaaaaaaaaaaaaa
aaaaaaaactgcaggcgtaccagctttccctatagtgagtcgtatta
TagO 998bp
gcatgcaattaaccctcactaaagagacgcgtacgtaagcttggatcctctagaCTCTGTGTCATGATCGTGA
GTTGTCGCAGTGTCTGTACCAATACTCTGGTGGAGCTATATAAGCCGCTGTTG
CGTAAATCAACGGCATGATCCCTATGACCGCGTCATGCTAACTGATACACGC
TGCTCGAACAGTGATACGCACACTGATAACTATGCGCAGACGCTTGAAACGA
TGTGACATCGCTTCTAGAGTATGAGCCGCAATGCACGACTGATACTCGATAT
GAGCAGCAGTCGGCTATGATTTGCAATGCTTGCAGTATGTATCCTGATCGTGC
GTGCGATGTCTGATAATACGCTCGCATGATATGTATTGCGCTCAGATGCTGGA
GATATGCCATGCGTGCTGTCAGTATGCCATGTATGCTGATATGTCGCGATCTA
TGTGGTGACTATGAGATCCATGTGATGACGTTGCAGTCTCTGTGACCTTATCG
ACGCGCATGTGAGCCTATAGACAGCGATGTGAGCACTCTCATCTGCGGATCA
GTCTATCCTCGCTGATGCTCAGTGATACACGCTGATGCACGTAGTGAGCATCC
TGTGCTCGCATATACCGCTGCTGCACTGATATGAGCCAGTGCTGCTGCTCTCT
ACGGAGTGTGCTCGGCTATAACAGCGAGTGCTACGCCTAAACTGGCTGTCTA
GCACTGTAGCTGGTGCATGTACTCGACTGCCGCTGCATCTACTATAAGACTCT
GACATTAGCGTATAGGCTGATACATTAGCTCGGATGCTATCAGCTTGCGCCTA
TTATATGCCTGACGCGGGATCTATCAGAACGACTCGGTAGCTCATATACTGG
ATCACGGTGCCACAACATGCTACACGAGGTCTCAGACTCTATCCCGTGGACT
CAACGTGCATCTGCTATGCTGAGCGCGTATCTGTGTACCTGTCCGATGCTCTG
ATCTACACTGCCGTGATCGTTATATGACGAGACTGTGCGCTCATAGCCGACAC
TGTGCTCGATAAGACCACGCTGTGCGGATATAgtcgacccgggaattccggaaaaaaaaaaaaa
aaaaaaaactgcaggcgtaccagctttccctatagtgagtcgtatta
TagQ 1000bp
gcatgcaattaaccctcactaaagggacgcgtacgtaagcttggatcctctagaCTAGTGCATCCTCGTGGCA
TCATGCGTCTCCTCAGTAGGTCTGCGACTGATCCTAGTGCAATGCGTCTGAGC
CTGAGCTACAGCGATATAGCCTGGATTGTGAGCGTATTTGCTGTCAGAACCTC
AGCTCATCATGTATGATGCTGTACCATCCTGCGATACTGAAGATGCACCGCTA
TAATGCGAGGCTCTCCGCTAAAGTGGAAGCTGCTCGTTCTCAATGCGAGCGA
GTCGAATCCAATGCCGTAGCTGCGATAACGATGCCGCTGACTCTACGGTAAT
GCACGATCCTCTACATTGATAGCAGATAGTCTAACGGGATAGCATAGGTGCA
AGGCTCCTAGCATGTAGTCACAGGTGCTCAGATATAGTCATCGCTGCAATCA
GCTAGTCATCTTGTCAGGATGCTACTCACTGCGTGCAGAAGATTCGCACGACT
TCAGAGGATGGCACTCGTCATTAGAGTGATGTTCTCGGATCGACACTGCTGGT
CTGCGAATGACTCGCATTCACTAACATGGAGCATCGTTATCTAAAGGGGATG
CACGTTATCGTCGAGTGGCCGTCATGTCTATGCAGTGCGGCCTATGTCTCATT
AGCGAGTCGTATGTATCATGTCGGGCTCGAATGTTGCACACGTCTGCGTAATG
GTGACCGCTAGTCCCASATGGTGCTTCGTAGCCACAAATGTCGTTAGGTAGAC
CGACGTTATCGCGCTATACCCGATGTCAACGCGAGTTAGACCGTATCGTCCCC
AGTGCCCTAAGATGGTCAAGCGTGCTCCTACGTTAGTATCAGTTTCCCTATTG
GTACGTCTGGCGTACTTCTGAAACGTGATGGGCGGCTGGTTACCCGTATATGG
GCTCGGTTGACCTCTATTGGGCGTTGTTGACCCGAATTCGGTATCCTCGTCGT
TAAATGGCGAACGTCGTCTGCTATAGGCAAACGTCTGTCGGTCATGGCAAAT
GTTACTCGTGTGTGCAAGAAATTACTCGCTGTCgtcgacccgggaattccggaaaaaaaaaaaa
aaaaaaaaactgcaggcgtaccagctttccctatagtgagtcgtatta
TagIN 1944bp
gcatgcaattaaccctcactaaagggacgcgtacgtaagcttGATAAGCGTTCACAGCTCGGCAATAC
CTGTGACGAGCTGCTCGCAAGATTTACGCAGTGTGGCTATACTTGACAGTGAT
GGCGCTTACTTCAGATGTATGGGTGATACTTCGCTATATGGGTGGTCACTTCT
CTATGGCGCGTGACAATGTACTATGGAGCGGTCAATGTCAGTACGGATCGCG
TCGATCTAGGTGACTACGCACGCCTCTGGAGTAAATCGAGTGCTCCGTGCGA
AATACGCGGTCATCGTGCGAATAACCGAGTCATCGTGAGTAGTATGAACGTG
TCGTGTTATGCAGCGGTATGTCGTGCTATAATGGCGTCTGTCGTGCTCATAAG
GTTCCTCTGATGTGCTAGACGTGTCCATCGAGCTGCATAGCTATACTTCGAGT
CACTTGGGATACTTCGATAGCGTTGTGAATAGTGTCGTAGGCTCTCGGGCACG
TTGTTAAACTGTTGCCGCCAATTCAAGATTAGTCCAGCTCGTACTATCGAATA
CACCATCGTCGTATCGAATAATCGCACCTCGTAGGAGTCAGTTGCCACTCGTT
GATAGTCAACCAAGCTCGTTAGATAGTAGCCCAGATCCTACGAGATGAGCTA
CGTAACTACAGTGATAGCATATAGGGTACGCTAGAATGCCAGGTCGTAGTCG
AATTAGTCAGGTTGGATGTCTACTAGTTGACTTGGAGTATGCCATGAAGACTC
GTCCCTCGATATCAATACTCGTCCGCAGGTGAACACTGTAGTCGGTGCTAGTG
CCCACTTCTCGGTATGTGTCCTCAATTATCGAGTAGGATTCTAATCAATCGTC
GCGGCTCACTAATTGTCTGCGGTGGCTACTAATGGTTACGGTGCCTGACTAAT
CGTGTAGGTGTCTAATACATCGTGATACGGGCGATATAATGCTCGATACGGC
AAATATAGCTCCGTCCGGTGGATCCAGATCGCAGGGTATCGCATCGACAGAC
CTGGTATCGTCGTGACGAACGTGCTACTCGCTTATCGGGCCTGCTACATCAGT
GGCGATGTTCGTAACCCTTAGCCGATCTTCTTACTTACGAGGCTACTATTCGA
TCAAACTCGCCTATCTGGTAATAACTGCGGTGATCTGGTAGCCACTACGTGCG
CCTGGTAGCAAATACGGCGAGCTGGTATCACTATCGGCTCAGTGGTCCGACA
TAGTGCCCAGTGGTTCGCATAACTGCCGCTGGGTCCAATATAACACGCAGTC
GTCAATCATACGAGCCGATGGTCAGCAATAGCGCCTGTGGTGACACTATGCC
ACCTCTGGTCTAATATAGCGCCCTGTGGTCGTATAATCGAGCGCGTAATCGTA
TATCCGACTGTAGGTGCGTAACTCGCGACTAGGTGGCTCTAATCTGCGTTGGT
TGTCGCTCACAGTGTCTGGTGTTCGATACCCGGATCGGGTTCCGTAATCTTGG
CATCGAGGTTTCGTACATGTCACGCGGTCTCGTTCATTCTCGGTGGTGCTCAG
TACATCCAGTGGTGAGTCGCTACATCACACGGTGATCCGGCTAAACCTCTGG
GCATCCGTATTAAGCGACATTCCTACGACTTATCAGCACGTCCTACGGTATAA
CAAGGCGTGCTACGGTCTAACGACGCTGGTAGCAGTCTATCAGATCGCTAGT
ACGAGTTAGAGATGCTTAGTACGCCTTCGAATCTATGATGCTCGTGCTCACGC
GATGCACTCGGATTATGGCACATGCACTCGCGTAATGACGCTGCATCGCTCA
GTATGATCCATGAGCGCCGTGAATGACGCATGAGCCTCGTATCGAGTGCATG
AGCTGTCTTTCACATGATACATCGCTCTAAATCATCATGCGACAGTCTCGACA
GCAGCTCAGCATCTATGCATCATGTGCCTCACTAGGACATCATGCTCGACTCT
GAGACACTGATCGAGCATTAAGACtctagagcggccgccgactagtgagctcgtcgaccccgggaatt
ccggaaaaaaaaaaaaaaaaaaaaactgcaggcgtaccagctttccctatagtgagtcgtatta
TagIQ (INOQ) 3849bp
gcatgcaattaaccctcactaaagggacgcgtacgtaagcttGATAAGCGTTCACAGCTCGGCAATAC
CTGTGACGAGCTGCTCGCAAGATTTACGCAGTGTGGCTATACTTGACAGTGAT
GGCGCTTACTTCAGATGTATGGGTGATACTTCGCTATATGGGTGGTCACTTCT
CTATGGCGCGTGACAATGTACTATGGAGCGGTCAATGTCAGTACGGATCGCG
TCGATCTAGGTGACTACGCACGCCTCTGGAGTAAATCGAGTGCTCCGTGCGA
AATACGCGGTCATCGTGCGAATAACCGAGTCATCGTGAGTAGTATGAACGTG
TCGTGTTATGCAGCGGTATGTCGTGCTATAATGGCGTCTGTCGTGCTCATAAG
GTTCCTCTGATGTGCTAGACGTGTCCATCGAGCTGCATAGCTATACTTCGAGT
CACTTGGGATACTTCGATAGCGTTGTGAATAGTGTCGTAGGCTCTCGGGCACG
TTGTTAAACTGTTGCCGCCAATTCAAGATTAGTCCAGCTCGTACTATCGAATA
CACCATCGTCGTATCGAATAATCGCACCTCGTAGGAGTCAGTTGCCACTCGTT
GATAGTCAACCAAGCTCGTTAGATAGTAGCCCAGATCCTACGAGATGAGCTA
CGTAACTACAGTGATAGCATATAGGGTACGCTAGAATGCCAGGTCGTAGTCG
AATTAGTCAGGTTGGATGTCTACTAGTTGACTTGGAGTATGCCATGAAGACTC
GTCCCTCGATATCAATACTCGTCCGCAGGTGAACACTGTAGTCGGTGCTAGTG
CCCACTTCTCGGTATGTGTCCTCAATTATCGAGTAGGATTCTAATCAATCGTC
GCGGCTCACTAATTGTCTGCGGTGGCTACTAATGGTTACGGTGCCTGACTAAT
CGTGTAGGTGTCTAATACATCGTGATACGGGCGATATAATGCTCGATACGGC
AAATATAGCTCCGTCCGGTGGATCCAGATCGCAGGGTATCGCATCGACAGAC
CTGGTATCGTCGTGACGAACGTGCTACTCGCTTATCGGGCCTGCTACATCAGT
GGCGATGTTCGTAACCCTTAGCCGATCTTCTTACTTACGAGGCTACTATTCGA
TCAAACTCGCCTATCTGGTAATAACTGCGGTGATCTGGTAGCCACTACGTGCG
CCTGGTAGCAAATACGGCGAGCTGGTATCACTATCGGCTCAGTGGTCCGACA
TAGTGCCCAGTGGTTCGCATAACTGCCGCTGGGTCCAATATAACACGCAGTC
GTCAATCATACGAGCCGATGGTCAGCAATAGCGCCTGTGGTGACACTATGCC
ACCTCTGGTCTAATATAGCGCCCTGTGGTCGTATAATCGAGCGCGTAATCGTA
TATCCGACTGTAGGTGCGTAACTCGCGACTAGGTGGCTCTAATCTGCGTTGGT
TGTCGCTCACAGTGTCTGGTGTTCGATACCCGGATCGGGTTCCGTAATCTTGG
CATCGAGGTTTCGTACATGTCACGCGGTCTCGTTCATTCTCGGTGGTGCTCAG
TACATCCAGTGGTGAGTCGCTACATCACACGGTGATCCGGCTAAACCTCTGG
GCATCCGTATTAAGCGACATTCCTACGACTTATCAGCACGTCCTACGGTATAA
CAAGGCGTGCTACGGTCTAACGACGCTGGTAGCAGTCTATCAGATCGCTAGT
ACGAGTTAGAGATGCTTAGTACGCCTTCGAATCTATGATGCTCGTGCTCACGC
GATGCACTCGGATTATGGCACATGCACTCGCGTAATGACGCTGCATCGCTCA
GTATGATCCATGAGCGCCGTGAATGACGCATGAGCCTCGTATCGAGTGCATG
AGCTGTCTTTCACATGATACATCGCTCTAAATCATCATGCGACAGTCTCGACA
GCAGCTCAGCATCTATGCATCATGTGCCTCACTAGGACATCATGCTCGACTCT
GAGACACTGATCGAGCATTAAGACTCTAGACTCTGTGCCATGATCGTGAGTT
GTCGCAGTGTCTGTACCAATACTCTGGTGGAGCTATATAAGCCGCTGTTGCGT
AAATCAACGGCATGATCCCTATGACCGCGTCATGCTAACTGATACACGCTGC
TCGAACAGTGATACGCACACTGATAACTATGCGCAGACGCTTGAAACGATGT
GACATCGCTTCTAGAGTATGAGCCGCAATGCACGACTGATACTCGATATGAG
CAGCAGTCGGCTATGATTTGCAATGCTTGCAGTATGTATCCTGATCGTGCGTG
CGATGTCTGATAATACGCTCGCATGATATGTATTGCGCTCAGATGCTGGAGAT
ATGCCATGCGTGCTGTCAGTATGCCATGTATGCTGATATGTCGCGATCTATGT
GGTGACTATGAGATCCATGTGATGACGTTGCAGTCTCTGTGACCTTATCGACG
CGCATGTGAGCCTATAGACAGCGATGTGAGCACTCTCATCTGCGGATCAGTC
TATCCTCGCTGATGCTCAGTGATACACGCTGATGCACGTAGTGAGCATCCTGT
GCTCGCATATACCGCTGCTGCACTGATATGAGCCAGTGCTGCTGCTCTCTACG
GAGTGTGCTCGGCTATAACAGCGAGTGCTACGCCTAAACTGGCTGTCTAGCA
CTGTAGCTGGTGCATGTACTCGACTGCCGCTGCATCTACTATAAGACTCTGAC
ATTAGCGTATAGGCTGATACATTAGCTCGGATGCTATCAGCTTGCGCCTATTA
TATGCCTGACGCGGGATCTATCAGAACGACTCGGTAGCTCATATACTGGATC
ACGGTGCCACAACATGCTACACGAGGTCTCAGACTCTATCCCGTGGACTCAA
CGTGCATCTGCTATGCTGAGCGCGTATCTGTGTACCTGTCCGATGCTCTGATC
TACACTGCCGTGATCGTTATATGACGAGACTGTGCGCTCATAGCCGACACTGT
GCTCGATAAGACCACGCTGTGCGGATATAGTCGACCTAGTGCATCCTCGTGG
CATCATGCGTCTCCTCAGTAGGTCTGCGACTGATCCTAGTGCAATGCGTCTGA
GCCTGAGCTACAGCGATATAGCCTGGATTGTGAGCGTATTTGCTGTCAGAAC
CTCAGCTCATCATGTATGATGCTGTACCATCCTGCGATACTGAAGATGCACCG
CTATAATGCGAGGCTCTCCGCTAAAGTGGAAGCTGCTCGTTCTCAATGCGAG
CGAGTCGAATCCAATGCCGTAGCTGCGATAACGATGCCGCTGACTCTACGGT
AATGCACGATCCTCTACATTGATAGCAGATAGTCTAACGGGATAGCATAGGT
GCAAGGCTCCTAGCATGTAGTCACAGGTGCTCAGATATAGTCATCGCTGCAA
TCAGCTAGTCATCTTGTCAGGATGCTACTCACTGCGTGCAGAAGATTCGCACG
ACTTCAGAGGATGGCACTCGTCATTAGAGTGATGTTCTCGGATCGACACTGCT
GGTCTGCGAATGACTCGCATTCACTAACATGGAGCATCGTTATCTAAAGGGG
ATGCACGTTATCGTCGAGTGGCCGTCATGTCTATGCAGTGCGGCCTATGTCTC
ATTAGCGAGTCGTATGTATCATGTCGGGCTCGAATGTTGCACACGTCTGCGTA
ATGGTGACCGCTAGTCCCACATGGTGCTTCGTAGCCACAAATGTCGTTAGGTA
GACCGACGTTATCGCGCTATACCCGATGTCAACGCGAGTTAGACCGTATCGT
CCCCAGTGCCCTAAGATGGTCAAGCGTGCTCCTACGTTAGTATCAGTTTCCCT
ATTGGTACGTCTGGCGTACTTCTGAAACGTGATGGGCGGCTGGTTACCCGTAT
ATGGGCTCGGTTGACCTCTATTGGGCGTTGTTGACCCgaattccggaaaaaaaaaaaaaaaa
aaaaactgcaggcgtaccagctttccctatagtgagtcgtatta
TagIQ.EX (3849 bp; the 2 bp differences from TagIQ are underlined and in bold)
gcatgcaattaaccctcactaaagggacgcgtacgtaagcttGATAAGCGTTCACAGCTCGGCAATAC
CTGTGACGAGCTGCTCGCAAGATTTACGCAGTGTGGCTATACTTGACAGTGAT
GGCGCTTACTTCAGATGTATGGGTGATACTTCGCTATATGGGTGGTCACTTCT
CTATGGCGCGTGACAATGTACTATGGAGCGGTCAATGTCAGTACGGATCGCG
TCGATCTAGGTGACTACGCACGCCTCTGGAGTAAATCGAGTGCTCCGTGCGA
AATACGCGGTCATCGTGCGAATAACCGAGTCATCGTGAGTAGTATGAACGTG
TCGTGTTATGCAGCGGTATGTCGTGCTATAATGGCGTCTGTCGTGCTCATAAG
GTTCCTCTGATGTGCTAGACGTGTCCATCGAGCTGCATAGCTATACTTCGAGT
CACTTGGGATACTTCGATAGCGTTGTGAATAGTGTCGTAGGCTCTCGGGCACG
TTGTTAAACTGTTGCCGCCAATTCAAGATTAGTCCAGCTCGTACTATCGAATA
CACCATCGTCGTATCGAATAATCGCACCTCGTAGGAGTCAGTTGCCACTCGTT
GATAGTCAACCAAGCTCGTTAGATAGTAGCCCAGATCCTACGAGATGAGCTA
CGTAACTACAGTGATAGCATATAGGGTACGCTAGAATGCCAGGTCGTAGTCG
AATTAGTCAGGTTGGATGTCTACTAGTTGACTTGGAGTATGCCATGAAGACTC
GTCCCTCGATATCAATACTCGTCCGCAGGTGAACACTGTAGTCGGTGCTAGTG
CCCACTTCTCGGTATGTGTCCTCAATTATCGAGTAGGATTCTAATCAATCGTC
GCGGCTCACTAATTGTCTGCGGTGGCTACTAATGGTTACGGTGCCTGACTAAT
CGTGTAGGTGTCTAATACATCGTGATACGGGCGATATAATGCTCGATACGGC
AAATATAGCTCCGTCCGGTGGATCCAGATCGCAGGGTATCGCATCGACAGAC
CTGGTATCGTCGTGACGAACGTGCTACTCGCTTATCGGGCCTGCTACATCAGT
GGCGATGTTCGTAACCCTTAGCCGATCTTCTTACTTACGAGGCTACTATTCGA
TCAAACTCGCCTATCTGGTAATAACTGCGGTGATCTGGTAGCCACTACGTGCG
CCTGGTAGCAAATACGGCGAGCTGGTATCACTATCGGCTCAGTGGTCCGACA
TAGTGCCCAGTGGTTCGCATAACTGCCGCTGGGTCCAATATAACACGCAGTC
GTCAATCATACGAGCCGATGGTCAGCAATAGCGCCTGTGGTGACACTATGCC
ACCTCTGGTCTAATATAGCGCCCTGTGGTCGTATAATCGAGCGCGTAATCGTA
TATCCGACTGTAGGTGCGTAACTCGCGACTAGGTGGCTCTAATCTGCGTTGGT
TGTCGCTCACAGTGTCTGGTGTTCGATACCCGGATCGGGTTCCGTAATCTTGG
CATCGAGGTTTCGTACATGTCACGCGGTCTCGTTCATTCTCGGTGGTGCTCAG
TACATCCAGTGGTGAGTCGCTACATCACACGGTGATCCGGCTAAACCTCTGG
GCATCCGTATTAAGCGACATTCCTACGACTTATCAGCACGTCCTACGGTATAA
CAAGGCGTGCTACGGTCTAACGACGCTGGTAGCAGTCTATCAGATCGCTAGT
ACGAGTTAGAGATGCTTAGTACGCCTTCGAATCTATGATGCTCGTGCTCACGC
GATGCACTCGGATTATGGCACATGCACTCGCGTAATGACGCTGCATCGCTCA
GTATGATCCATGAGCGCCGTGAATGACGCATGAGCCTCGTATCGAGTGCATG
AGCTGTCTTTCACATGATACATCGCTCTAAATCATCATGCGACAGTCTCGACA
GCAGCTCAGCATCTATGCATCATGTGCCTCACTAGGACATCATGCTCGACTCT
GAGACACTGATCGAGCATTAAGACTCTAGACTCTGTGCCATGATCGTGAGTT
GTCGCAGTGTCTGTACCAATACTCTGGTGGAGCTATATAAGCCGCTGTTGCGT
AAATCAACGGCATGATCCCTATGACCGCGTCATGCTAACTGATACACGCTGC
TCGAACAGTGATACGCACACTGATAACTATGCGCAGACGCTTGAAACGATGT
GACATCGCTTCTAGAGTATGAGCCGCAATGCACGACTGATACTCGATATGAG
CAGCAGTCGGCTATGATTTGCAATGCTTGCAGTATGTATCCTGATCGTGCGTG
CGATGTCTGATAATACGCTCGCATGATATGTATTGCGCTCAGATGCTGGAGAT
ATGCCATGCGTGCTGTCAGTATGCCATGTATGCTGATATGTCGCGATCTATGT
GGTGACTATGAGATCCATGTGATGACGTTGCAGTCTCTGTGACCTTATCGACG
CGCATGTGAGCCTATAGACAGCGATGTGAGCACTCTCATCTGCGGATCAGTC
TATCCTCGCTGATGCTCAGTGATACACGCTGATGCACGTAGTGAGCATCCTGT
GCTCGCATATACCGCTGCTGCACTGATATGAGCCAGTGCTGCTGCTCTCTACG
GAGTGTGCTCGGCTATAACAGCGAGTGCTACGCCTAAACTGGCTGTCTAGAA
CTGTAGCTGGTGCATGTACTCGACTGCCGCTGCATCTACTATAAGACTCTGAC
ATTAGCGTATAGGCTGATACATTAGCTCGGATGCTATCAGCTTGCGCCTATTA
TATGCCTGACGCGGGATCTATCAGAACGACTCGGTAGCTCATATACTGGATC
ACGGTGCCACAACATGCTACACGAGGTCTCAGACTCTATCCCGTGGACTCAA
CGTGCATCTGCTATGCTGAGCGCGTATCTGTGTACCTGTCCGATGCTCTGATC
TACACTGCCGTGATCGTTATATGACGAGACTGTGCGCTCATAGCCGACACTGT
GCTCGATAAGACCACGCTGTGCGGATATAGTCGACCTAGTGCATCCTCGTGG
CATCATGCGTCTCCTCAGTAGGTCTGCGACTGATCCTAGTGCAATGCGTCTGA
GCCTGAGCTACAGCGATATAGCCTGGATTGTGAGCGTATTTGCTGTCAGAAC
CTCAGCTCATCATGTATGATGCTGTACCATCCTGCGATACTGAAGATGCACCG
CTATAATGCGAGGCTCTCCGCTAAAGTGGAAGCTGCTCGTTCTCAATGCGAG
CGAGTCGAATTCAATGCCGTAGCTGCGATAACGATGCCGCTGACTCTACGGT
AATGCACGATCCTCTACATTGATAGCAGATAGTCTAACGGGATAGCATAGGT
GCAAGGCTCCTAGCATGTAGTCACAGGTGCTCAGATATAGTCATCGCTGCAA
TCAGCTAGTCATCTTGTCAGGATGCTACTCACTGCGTGCAGAAGATTCGCACG
ACTTCAGAGGATGGCACTCGTCATTAGAGTGATGTTCTCGGATCGACACTGCT
GGTCTGCGAATGACTCGCATTCACTAACATGGAGCATCGTTATCTAAAGGGG
ATGCACGTTATCGTCGAGTGGCCGTCATGTCTATGCAGTGCGGCCTATGTCTC
ATTAGCGAGTCGTATGTATCATGTCGGGCTCGAATGTTGCACACGTCTGCGTA
ATGGTGACCGCTAGTCCCACATGGTGCTTCGTAGCCACAAATGTCGTTAGGTA
GACCGACGTTATCGCGCTATACCCGATGTCAACGCGAGTTAGACCGTATCGT
CCCCAGTGCCCTAAGATGGTCAAGCGTGCTCCTACGTTAGTATCAGTTTCCCT
ATTGGTACGTCTGGCGTACTTCTGAAACGTGATGGGCGGCTGGTTACCCGTAT
ATGGGCTCGGTTGACCTCTATTGGGCGTTGTTGACCCgaattccggaaaaaaaaaaaaaaaa
aaaaactgcaggcgtaccagctttccctatagtgagtcgtatta

### Example 2

Testing the Tag Genes

The synthetic genes were tested in a number of ways. 1) An oligonucleotide array was designed and made to probe many positions along the length of each Tag gene. Hybridizing RNA made from the Tag genes clearly shows the expected uniform hybridization both across each gene and between the 13 genes, a uniformity that is lacking from naturally occurring genes. This uniformity is expected because the Tags are originally designed for such characteristic.

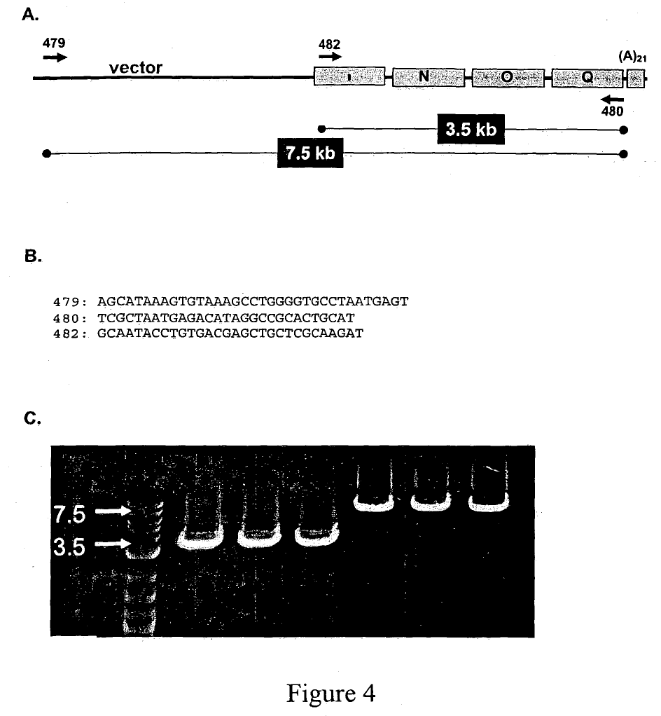

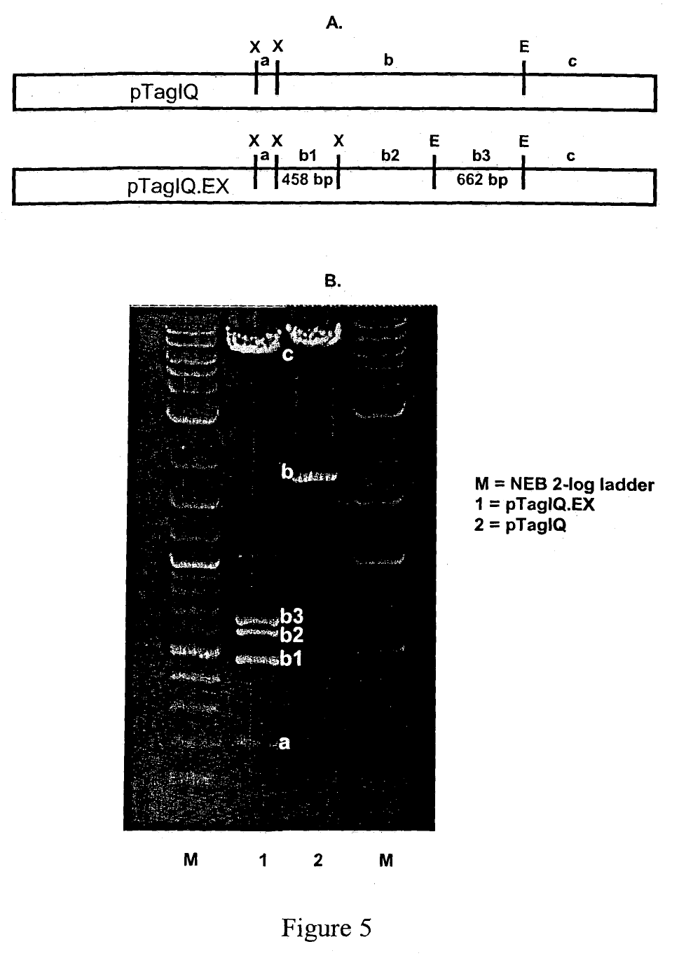

In addition, the average signal from the Tag genes is higher than the signal from transcripts from human genes spiked in at equivalent concentrations. Data from these experiments are used to help develop new probe selection rules and new gene expression algorithms. 2) Probe sets for the Tag genes are included on the Affymetrix HG^{—}U133 human gene expression arrays (Affymetrix, Inc., Santa Clara, Calif.). Tag gene RNA spikes are used to help validate the array design. Again the Tag gene transcripts demonstrate consistent hybridization and high signal intensity. 3) The plasmid containing the longest Tag gene construct, pTagIQ, contains 3849 bp of Tag sequence (Tags I, N, O, and most of Q). This plasmid may be used for genotyping applications. For variant detection (resequencing) assays, the plasmid may be used as a template to test long-range PCR (FIG. 4) and the PCR product from this plasmid can be labeled and hybridized to test other steps of the assay. For microarray SNP analysis, TagIQ.EX (FIG. 5) can serve as an assay control. One sample preparation method calls for digesting genomic DNA with a restriction endonuclease and then preferentially amplifying fragments of a particular size range, 400-800 bp, for example. TagIQ.EX can be added to the test DNA, and then digested with XbaI or EcoRI, amplified, labeled, and hybridized along with the test DNA. The results of the Tag sequence can be used to assess system performance. 4) RNA spikes from Tag genes have been used as exogenous controls in quantitative RT-PCR experiments. These spikes can be used to normalize quantitative RT-PCR to aid in determining absolute transcript levels. In addition, the Tag gene spikes can also allow direct comparisons between microarray and RT-PCR results, or between different types of microarrays (spotted arrays vs. GeneChip® arrays (Affymetrix, Inc., Santa Clara, Calif.), for example). The universal absence of the synthetic genes will also allow comparisons between different sample types; for example, data from microarray and RT-PCR experiments can be normalized for samples from mouse, human, and bacteria.

An example of an application of the cloned Tag genes is provided by the Affymetrix CustomSeq™ resequencing arrays, which contain probes complementary to portions of both DNA strands of the TagIQ.EX sequence, as well as probes complementary to DNA derived from customer-specified genes or genomes. A GeneChip® Resequencing Assay Kit containing the TagIQ.EX plasmid and PCR primers is available from Affymetrix to amplify the relevant Tag DNA, and thus serves as a control for the PCR process. Amplified Tag DNA can then serve as a control for fragmentation and labeling. Furthermore, because the Tag sequence was chosen to be absent from any genomic sample, cross-hybridization should be minimal between Tag-derived DNA and DNA derived from any genomic sample, so Tag DNA can be mixed with DNA complementary to other probes on the resequencing arrays. Hybridization of the mixture to resequencing arrays provides a control of the hybridization and base-calling process.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by references for all purposes.

What is claimed is:

1. A DNA molecule comprising the following elements in a 5′ to 3′ direction:
a first restriction endonuclease site,
a T3 promoter site;
at least one Tag gene, said Tag gene comprising at least 5 20 mer Tag sequences;
a Poly A site having at least 21 consecutive A residues, wherein said A residues are on the same strand as said T3 promoter such that when transcription is initiated at the T3 promoter, a Tag RNA transcript is produced having a poly A tail.
a second restriction endonuclease site which may be the same or different than said first restriction endonuclease site;
a T7 Promoter on the opposite strand as said T3 promoter.

2. A DNA molecule according to claim 1 wherein said Tag sequences are selected from Seq. Id. Nos. 1-2050 or their complement.

3. A DNA molecule according to claim 1 wherein said Tag gene is selected from the group consisting of Tags A, B, C, D, E, F, G, H, I, J, N, O, Q, Tag IN, Tag IQ and Tag IQ.EX.

4. A DNA molecule according to claim 1 wherein, said first restriction endonuclease site is SphI (gcatgc), said T3 promoter comprises the following sequence aattaaccctcactaaagg; said Tag gene is selected from the group consisting of Tags A, B, C, D, E, F, G, H, I, J, N, O, Q, Tag IN, Tag IQ and Tag IQ.EX; said second endonuclease site comprises a PstI site (ctgcag); and said T7 promoter comprises tatagtgagtcgtatta.

5. A DNA molecule according to claim 1 comprising the sequence, wherein capitalized bases refer to Tag gene sequence:
gcatgcaattaaccctcactaaagggacgcgtacgtaagcttggatcctctagaATTTGATCGTAACTCGGGT
GACCAATGACCATATACGGCGTATTAAGGTTGTACCCTCGGTCTCAACTTGTC
GTATGGGACTTTCAAGTACCTTAGCTCGTCGGACGCTTTAGATGACTTATCCA
TAGTCCTAAGTCCGGCGCCGGTTAAGCCGCTATTAGCGTGTGTGGACTCTCTC
TAGGAGCGGCTTCGCACAAATTACTGCTCAATCCTAGATACGTTGCGCTCTTT
GGTAAACGGCTCAGATCTTAGCACTCGTGCAGTTCTACGATGGCAAGTCGTG
CCTCGTTCTCGTGTAGAATATCAGCTAATAGGGTCGGCTCAACAGTGTATCCG
GTGGACAAGCACTGACACGCGATGACGTTCGTCAAGAGTCGCATAATCTCAG
AATCCGTACAGCCGCATCGGGTTCACGGCTATAAAACAGCGTCATCAGCGTA
GGGTATCGCTTCGCGTGTCATGACTTGGGCCACGTCTCTCTCTCGCACATTAG
GCTAGATTgtcgacccgggaattccggaaaaaaaaaaaaaaaaaaaaactgcagcgtaccagctttccctatagtgagt
cgtatta.

6. A DNA molecule according to claim 1 comprising the sequence, wherein capitalized bases refer to Tag gene sequence:
gcatgcaattaaccctcactaaagggacgcgtacgtaagcttggatcctctagaTTTAGTCGTTAGCCCGAGC
TTAACTATTAGCGTCGGTGCTATATCCTTACCGCGTATGGAGTAGCCTTCCCG
AGCATTTGTCTACCTTACCGTCAAGAAAACCATCGACTCACGGGATATTGACC
AAACTGCGGTGCGATTAACTCGACTGCCGCGTGAACAACGATGAGACCGGGC
TAAGGCACGTATCATATCCCTAATTCGCTGAATAGTGCCCTACATATCCTAAT
ACAGGCGCGACGAACCTTATACTCGATGGAAGACAGTTATACCCATGCATAA
AGCTCTATACTCCGAGAACTAGCATCTAAGCACTCGGCTCTAATGTTAAGTGC
TCGACCACAGATCGAAGGTCGGAACTCCAGTGCCAAGTACGATGGCTCACGT
CTTATTTGGGCCGCCAGAGTTATGTTTGAGTCTTCGATGTATGCGCTCGTTGC
CCTATTGTTGTGTCGGATCTTCTAGTTgtcgacccgggaattccggaaaaaaaaaaaaaaaaaaaaac
tgcaggcgtaccagctttccctatagtgagtcgtatta.

7. A DNA molecule according to claim 1 comprising the sequence, wherein capitalized bases refer to Tag gene sequence:
gcatgcaattaaccctcactaaagggacgcgtacgtaagcttggatcctctagaTGTGATAATTTCGACGAGG
CGTTACATATTCTGAGAGGGGTGATTAAGTCTGCTTCGGCCTGGGATGGTCTG
TCTACGTGTGCGTAGTTCTGTCATAGCGTCGAGGATTCTGAACCTGTCCATAG
TATCCTGTAAGCGTCCAATGTACCTATATCGTGGACCCAAAGTCGATACGTCC
GATTAAGCGACGTTGGTCTAGGTAACGAATTATACCCTCGGCTTACGAATTAT
GGCTGTGCCTAACGAATCTGGGACGTGCCTAAGTAATCTGGTCCGCGACTAA
GATGTACGGTGATCGTGGACGCTTGACCGGACTTATGCGTCGCCTTCCGAGTT
ATTGGATGGCGTTCCGTCCTATTGGATACTATTCCGTGCGTGTGCGACACGTT
CCGAGCATATGCTAACAGTTCCGTCACTATGTAACGCTTGACGTAGATTGCTA
TCAGGTTACGATGACTGCTAAGCCATTACGCGACATTCTGCAAAGTTACGTCG
CATTCTCTCACGTTACGGCTGATTCTCTAGGCTTACGCGCATGAGCTCTAGGT
TCCGGGTACTATCGAACGTGTCATTGGTACTgtcgacccgggaattccggaaaaaaaaaaaaaaa
aaaaaactgcaggcgtaccagctttccctatagtgagtcgtatta.

8. A DNA molecule according to claim 1 comprising the sequence, wherein capitalized bases refer to Tag gene sequence:
gcatgcaattaaccctcactaaagggacgcgtacgtaagcttggatcctctagaATAGACTAGCCTGCCGGTC
AATAACTGATGACGCGGAGTCAACCTGATAACCCATAGCGGAACAGTCTAAC
CTACGCGAGATACGTCTTACCGCACATAGGTAACCTATTCGTGACTAGCAGG
CCTTATTCCGGTGCTATGAGTATCTTACCTGGTCTAGGTATCTAATTCGTGAG
TCGGGTACTACATTCGTGCGATGGGTCCTCGCTTCGTCTATGAGGTCTCGTCT
TCGTGAGTGCAATGTATCCGAAGTCGTAGTGATAATATGGAACTAGGCGCGA
TTTGACGAACGTATGCCGCATATTCGGAACGTCGCCTGGAAATTCGCCACCTA
GATCGAAATTATCGGAACTCGTCGCTTATTTACGAACCTTGGGAGCCGTTCCT
AAAGCTGAGTCTGGTTTCTTATTAGCGAGGAGCATTTCGTGAATACTGAGCCG
AATATCGTAAGACATCCGCGAGCGACTGTAAACTAATCGGGGAACTTATTAT
AGAGCCGGTCCAGGTCTTGAACGACGTgtcgacccgggaattccggaaaaaaaaaaaaaaaaaaaa
actgcaggcgtaccagctttccctatagtgagtcgtatta.

9. A DNA molecule according to claim 1 comprising the sequence, wherein capitalized bases refer to Tag gene sequence:
gcatgcaattaaccctcactaaagggacgcgtacgtaagcttggatcctctagaCCATCCGATTAAATACCGT
GGATTACGTTAAGTTACGGCGGTTGACTTAGTTATGCGAGGTTCGCTTACGTT
GCATAGCGGATCGCTTAACCTCTATGCGTACAGCTTACCTACTATGCGTGCAA
GTTACCGAGCTGACGTCGCGTTAGACAGCTCATTCGTCACGTTTAGGACTATG
TCGAAGCGTTTCGACCATGTCGTCTAGCTTAATACCTCTGCGTCTCAGTTAAT
AGTACGGGCAATCCGTTATGTAAAGGGTGACCACGTTTCAGAAGCTGCCATA
TACTTACACAGCAGGCGATCACGTTAGATCCACTGCGTCACGTTACCTACATG
ATCGATCCGATTACAGGCCGATCCATCGGATTACACACGAGTCCTGCACGTT
AGAACACTGGCTCGCGGCTTAGATCAGCTTCCCTCGCTGGAGATCGAATACG
CCCAGCTWAGAGCGAATTGCGGCGCGTTCGACATAATTGCCGACGCTTCGAC
AGAATTGTAGGCGATTCTAGCCAATTGCACGTCGTATTAGGTAGTCACTCTCG
ACCTAGCGTAAGGATCCACGATCCTAGAGTCGGgtcgacccgggaattccggaaaaaaaaaaa
aaaaaaaaaactgcaggcgtaccagctttccctatagtgagtcgtatta.

10. A DNA molecule according to claim 1 comprising the sequence, wherein capitalized bases refer to Tag gene sequence:
gcatgcaattaaccctcactaaagggacgcgtacgtaagcttggatcctctagaACGCGGTCACTCAGCATAT
AGTCGTTGCACCTAGTTGATAGTCGCCGATTCTAGTTATGGCGTCGGATTAGA
CCGGATCACCCGGACATGGACGTTAAGTATCCGGCCTGGACGACAATAATTC
GGCGGTGCCTCACAATATTCCGAGAACTCTGCATCAATTCGGGCTAGTCGTAC
CTGAACGGGCATCAGTCGAATCTCTTCGTGGCTAGTCTGTGACGTCCGTGGTT
CATCGTGTCACCACGCGGTACATGAGTCAAAGTCCGAATAGCTCGCGCAACG
TCCGTCTAGCTGGATCAACCTATCCCTGAGTCTATATGCGTACCAATGGATGC
GGTCTCCTCCGACTGAGTATGCGTTCCTCGGACTGGATCAGCTATCCACGAGC
TGTAATCCGGTACTAGGGTGTATCGCCTGTTACTAGGTTAGACAGTCGTGTAC
TCGGTTAGACTGATGGTCAACGACCTATACTGACAGCATACGAGACGTGACG
ACTGCATAGTGGTCGGTCTGACACATCTCCTCGTTGGTAGTACGTGCCCCGTA
TGGATAGGGCTCTAGCCCGCTATGGTCAGTCTAATCGCCGTTGGTCTGTATGC
AGTGCGGTATGGTTCCTCTCAGTCACGTATGGTTCGCTGCTGTCCGTCATGTG
TTAGATGCgtcgacccgggaattccggaaaaaaaaaaaaaaaaaaaaactgcaggcgtaccagctttccctatagtgag
tcgtatta.

11. A DNA molecule according to claim 1 comprising the sequence, wherein capitalized bases refer to Tag gene sequence:
gcatgcaattaaccctcactaaagggacgcgtacgtaagcttggatcctctagaATGCAGCGTAGGTATCGAC
TCTCACTGTGGAGTCGTCTATGATGTCGTGGAGTCCTCTCAGAGTGCTGTAGG
TCCTCATAGGTCGTGCTGTCTCTCTACACGCGTGCGTGAGTCTACATTTCTGC
GAGTTGGTGCTCTCACTGCGGTGTCAGTGATCTCTCCGCGTGTGACATGAGTC
TAGCTTCGCGGTCATGGTCTATCCCAGCGATGGATGAGACTACTCTGTACTAG
ATGGTCATGCCTGCGAATGAGTCGTCAGTGCCCACAATGTCTCGATAGTGCG
CCGAATGTGTCTGTAATGCCTCGAATGTGTAATCGTCAACTCGTATGTGAAGT
GCTAGGCTAGTATTGACATCTACGGGCGGCTATTGACGAACTCTCCGGTATAT
GCTCTACATCTGCAGGGAATTGCCGACCATATATGGGTCTTGCTGATACGCTA
GGGTGCTTGCTACTTAGATAGGCGTCTTGGCCGCTATTCGCGGCGTGTCTCAG
AATATGCGCGACGTGTCTGGTATATGGCGACTGTGTCCGTCTATACGCATACT
GGTCCACATATAGACATACTTCCACGACATGACAAAGCGTGCTCCTACATAG
CACGAGCGTCTCCTAAATAGATCCGGTCTTATCGCTGAATGTCTAGGATTCTC
GTCAATGATCTACGATCCTCGCTAAGTATTCAGCCACCTCGTATAGTATTCGC
GCACCTGAGGATTTATTCACCTGACTCGCGTATAATATGCCGTCACCTAGTCT
Agtcgacccgggaattccggaaaaaaaaaaaaaaaaaaaaactgcaggcgtaccagctttccctatagtgagtcgtatta.

12. A DNA molecule according to claim 1 comprising the sequence, wherein capitalized bases refer to Tag gene sequence:
gcatgcaattaaccctcactaaagggacgcgtacgtaagcttggatcctctagaGATATGCGTTACGTGAGTC
TGATAGCAGTTCACTACCTGGATATCTGATCCACTAGCTCGATCATGCTCACC
CATAGTTTATCTGCATCACTCGTACTGAAATGCTCACATCGCAGGTAGAGCAG
CATCGTAGAGCGTCAAGCTGCATCCTAGCGTCATGAGTCATAGTACCTCATGC
TCACGTGATCTACCCTAGCTGACCGCTAATGACGGCAGTGCAACCTGAGATA
CCGACGGCATACTGTCGTCAACGTCAGGCAATGTGTCCGAACGGCGAGCTAC
GTCGCCTCACGGAGTAATCGCGTCCCTCTAGGTATAGTGCCGTCGGTTCAGGT
CATATGTCGCGGGTTCTGCACATATCACGGACGTATCGCTATCAGACGGACG
CTCTCGGACCTAAACCGTAGCTCTCGGCAAGATCGTCCTCGTCTCGAATATAG
CGCCCTAGTGCTGCAAATGTCACCGCTATCTCGTAAGGGGTCCGTCTGTTGAG
TAGGCCTCCTCTCGTTGGATGTGAGCTCGGTTGCTTGGATGGTGCAGCTTAC
TTCGCGTACCTGCTGTTTGCATCAGTCCTCTGCATCTATAATCGCGTATCTCTC
TCTAGTAGACCATATAGCCATCTAAGCGCTCGATATTCCACCTAAGTGGCGCC
TATTGAACTAAGTGGCAGCCGAATGGACTATCGCTCCTCGATATGTACGGAT
AGGCCACGGCATGTACGAGCATAAGCCGAACTGCACGAGCATACCCGACACT
GATCTGAGAGTCGCTTAAATCATCTGCGTGTCTTAGAGCTTATCGCCATGTCT
GTCAACTGTACTGTCATCCTGTAACTGTAGCGTATGTGgtcgacccgggaattccggaaaa
aaaaaaaaaaaaaaaaactgcaggcgtaccagctttccctatagtgagtcgtatta.

13. A DNA molecule according to claim 1 comprising the sequence, wherein capitalized bases refer to Tag gene sequence:
gcatgcaattaaccctcactaaagggacgcgtacgtaagcttggatcctctagaGATAAGCGTTCACAGCTCG
GCAATACCTGTGACGAGCTGCTCGCAAGATTTACGCAGTGTGGCTATACTTG
ACAGTGATGGCGCTTACTTCAGATGTATGGGTGATACTTCGCTATATGGGTGG
TCACTTCTCTATGGCGCGTGACAATGTACTATGGAGCGGTCAATGTCAGTACG
GATCGCGTCGATCTAGGTGACTACGCACGCCTCTGGAGTAAATCGARWGCTC
CGTGCGAAATACGCGGTCATCGTGCGAATAACCGAGTCATCGTGAGTAGTAT
GAACGTGTCGTGTTATGCAGCGGTATGTCGTGCTATAATGGCGTCTGTCGTGC
TCATAAGGTTCCTCTGATGTGCTAGACGTGTCCATCGAGCTGCATAGCTATAC
TTCGAGTCACTTGGGATACTTCGATAGCGTTGTGAATAGTGTCGTAGGCTCTC
GGGCACGTTGYTAAACTGTTGCCGCCAATTCAAGATTAGTCCAGCTCGTACTA
TCGAATACACCATCGTCGTATCGAATAATCGCACCTCGTAGGAGTCAGTTGCC
ACTCGTTGATAGTCAACCAAGCTCGTTAGATAGTAGCCCAGATCCTACGAGA
TGAGCTACGTAACTACAGTGATAGCATATAGGGTACGCTAGAATGCCAGGTC
GTAGTCGAATTAGTCAGGTTGGATGTCTACTAGTTGACTTGGAGTATGCCATG
AAGACTCGTCCCTCGATATCAATACTCGTCCGCAGGTGAACACTGTAGTCGGT
GCTAGTGCCCACTTCTCGGTATGTGTCCTCAATTATCGAGTAGGATTCTAATC
AATCGTCGCGGCTCACTAATYGTCTGCGGTGGCTACTAATGGTTACGGTGCCT
GACTAATCGTGTAGGTGTCTAATACATCGTGATACGGGCGATATAATGCTCG
ATACGGCAAATATAGCTCCGTCCGGTgtcgacccgggaattccggaaaaaaaaaaaaaaaaaaaaac
tgcaggcgtaccagctttccctatagtgagtcgtatta.

14. A DNA molecule according to claim 1 comprising the sequence, wherein capitalized bases refer to Tag gene
sequence: gcatgcaattaaccctcactaaagggacgcgtacgtaagcttggatcctctagaCAATGATAGGCTA
GTCTCGCGCAGTACATGGTAGTTCAGCCAATAGATGCCTAGTACGCTGACGG
CATTCAGAGTACGCTGATCGGCTTATGACGTATGTGACGCAGCTCTTAGCGCA
ATGTATGTGCTGTTATCGAAGCCTATGGCTGAGTATGTAACGCTATGGCGTGC
TAGTCGTCTCATATACGTCTGATGACCTCGTATCATGTTATAGGGCTGCGAAC
TGTCGATGATGGTCACGACTCTGTCGATAGCTGTGTGACTCATTCAGAAGGTG
TGCAGCCTATATGATACGCAGTCGCATCCTATCTTACGTGTCAGTACTATGTG
TGAGTGCTCCGCCCTAGTGCTGATGTATGCCCCATAGTGCTCAGTGGAGTCTC
TCTTAGCATAGTGTCCGCTCATACATTAGATGGACGGCTCATTAGTATCATCG
TCGGCTGATATAGGTCGTGGCTCCCTGTATATCGAGGTGAGTCTATCTGGATC
AACGTCGCACTATGATGTGCAAAGTGTCGTCCATGTATAGACAGTGCGCGTA
TCATATAGGATGCGGCGATCTCATACAGCGTTACGGTCGCTGCGTACTGTATA
AGGATGCTCTGTGAACTGTCATCGGTCCGATCAATTAGTCTAGTGTGCGTTAT
TCAGATCGAGTGAGTACATGATTCGTCAGTGTGGATCAATTACAGTTAGGCC
GCTGACACATTAGTAACGTCGGCAAGCACTTAGTCGTGTCGTAAGCCAGTGT
GTCGTGTCTTAGACGACTGTGTGTGATTCTCGAGCGATTTATACATCCGTGAC
AGCGTTTATAGTGTGCTGACAGACTGGTTGGTTATCCAATGATCGACCTGGAG
TCTAATATCTGACCACGCCTTGTAATCGTATGACACGCGCTTGACACGACTGA
ATCCAGCTTAAGAGCCCTGCAACGCGATATACAGGCGCTGCTACCGATATgtcg
acccgggaattccggaaaaaaaaaaaaaaaaaaaaactgcaggcgtaccagctttccctatagtgagtcgtatta.

15. A DNA molecule according to claim 1 comprising the sequence, wherein capitalized bases refer to Tag gene
sequence: gcatgcaattaaccctcactaaagggacgcgtacgtaagcttggatcctctagaAGATCGCAGGGTA
TCGCATCGACAGACCTGGTATCGTCGTGACGAACGTGCTACTCGCTTATCGGG
CCTGCTACATCAGTGGCGATGTTCGTAACCCTTAGCCGATCTTCTTACTTACG
AGGCTACTATTCGATCAAACTCGCCTATCTGGTAATAACTGCGGTGATCTGGT
AGCCACTACGTGCGCCTGGTAGCAAATACGGCGAGCTGGTATCACTATCGGC
TCAGTGGTCCGACATAGTGCCCAGTGGTTCGCATAACTGCCGCTGGGTCCAAT
ATAACACGCAGTCGTCAATCATACGAGCCGATGGTCAGCAATAGCGCCTGTG
GTGACACTATGCCACCTCTGGTCTAATATAGCGCCCTGTGGTCGTATAATCGA
GCGCGTAATCGTATATYCGACTGTAGGTGCGTAACTCGCGACTAGGTGGCTC
TAATCTGCGTTGGTTGTCGCTCACAGTGTCTGGTGTTCGATACCCGGATCGGG
TTCCGTAATCTTGGCATCGAGGTTTCGTACATGTCACGCGGTCTCGTTCATTCT
CGGTGGTGCTCAGTACATCCAGTGGTGAGTCGCTACATCACACGGTGATCCG
GCTAAACCTCTGGGCATCCGTATTAAGCGACATTCCTACGACTTATCAGCACG
TCCTACGGTATAACAAGGCGTGCTACGGTCTAACGACGCTGGTACCAGTCTA
TCAGATCGCTAGTACGAGTTAGAGATGCTTAGTACGCCTTCGAATCTATGATG
CTCGTGCTCACGCGATGCACTCGGATTATGGCACATGCACTCGCGTAATGAC
GCTGCATCGCTCAGTATGATCCATGAGCGCCGTGAATGACGCATGAGCCTCG
TATCGAGTGCATGAGCTGTCTTTCACATGATACATCGCTCTAAATCATCATGC
GACAGTCTCGACAGCAGCTCAGCATCTATGCATCATGTGCCTCACTAGGACA
TCATGCTCGACTCTGAGACACTGATCGAGCATTAAGACgtcgacccgggaattccggaaaa
aaaaaaaaaaaaaaaaactgcaggcgtaccagctttccctatagtgagtcgtatta.

16. A DNA molecule according to claim 1 comprising the sequence, wherein capitalized bases refer to Tag gene
sequence:gcatgcaattaaccctcactaaagagacgcgtacgtaagcttggatcctctagaCTCTGTGTCATGAT
CGTGAGTTGTCGCAGTGTCTGTACCAATACTCTGGTGGAGCTATATAAGCCGC
TGTTGCGTAAATCAACGGCATGATCCCTATGACCGCGTCATGCTAACTGATAC
ACGCTGCTCGAACAGTGATACGCACACTGATAACTATGCGCAGACGCTTGAA
ACGATGTGACATCGCTTCTAGAGTATGAGCCGCAATGCACGACTGATACTCG
ATATGAGCAGCAGTCGGCTATGATTTGCAATGCTTGCAGTATGTATCCTGATC
GTGCGTGCGATGTCTGATAATACGCTCGCATGATATGTATTGCGCTCAGATGC
TGGAGATATGCCATGCGTGCTGTCAGTATGCCATGTATGCTGATATGTCGCGA
TCTATGTGGTGACTATGAGATCCATGTGATGACGTTGCAGTCTCTGTGACCTT
ATCGACGCGCATGTGAGCCTATAGACACJCGATGTGAGCACTCTCATCTGCGG
ATCAGTCTATCCTCGCTGATGCTCAGTGATACACGCTGATGCACGTAGTGAGC
ATCCTGTGCTCGCATATACCGCTGCTGCACTGATATGAGCCAGTGCTGCTGCT
CTCTACGGAGTGTGCTCGGCTATAACAGCGAGTGCTACGCCTAAACTGGCTG
TCTAGCACTGTAGCTGGTGCATGTACTCGACTGCCGCTGCATCTACTATAAGA
CTCTGACATTAGCGTATAGGCTGATACATTAGCTCGGATGCTATCAGCTTGCG
CCTATTATATGCCTGACGCGGGATCTATCAGAACGACTCGGTAGCTCATATAC
TGGATCACGGTGCCACAACATGCTACACGAGGTCTCAGACTCTATCCCGTGG
ACTCAACGTGCATCTGCTATGCTGAGCGCGTATCTGTGTACCTGTCCGATGCT
CTGATCTACACTGCCGTGATCGTTATATGACGAGACTGTGCGCTCATAGCCGA
CACTGTGCTCGATAAGACCACGCTGTGCGGATATAgtcgacccgggaattccggaaaaaaaa
aaaaaaaaaaaaactgcaggcgtaccagctttccctatagtgagtcgtatta.

17. A DNA molecule according to claim 1 comprising the sequence, wherein capitalized bases refer to Tag gene
sequence:gcatgcaattaaccctcactaaagggacgcgtacgtaagcttggatcctctagaCTAGTGCATCCTCG
TGGCATCATGCGTCTCCTCAGTAGGTCTGCGACTGATCCTAGTGCAATGCGTC
TGAGCCTGAGCTACAGCGATATAGCCTGGATTGTGAGCGTATTTGCTGTCAG
AACCTCAGCTCATCATGTATGATGCTGTACCATCCTGCGATACTGAAGATGCA
CCGCTATAATGCGAGGCTCTCCGCTAAAGTGGAAGCTGCTCGTTCTCAATGCG
AGCGAGTCGAATCCAATGCCGTAGCTGCGATAACGATGCCGCTGACTCTACG
GTAATGCACGATCCTCTACATTGATAGCAGATAGTCTAACGGGATAGCATAG
GTGCAAGGCTCCTAGCATGTAGTCACAGGTGCTCAGATATAGTCATCGCTGC
AATCAGCTAGTCATCTTGTCAGGATGCTACTCACTGCGTGCAGAAGATTCGCA
CGACTTCAGAGGATGGCACTCGTCATTAGAGTGATGTTCTCGGATCGACACT
GCTGGTCTGCGAATGACTCGCATTCACTAACATGGAGCATCGTTATCTAAAG
GGGATGCACGTTATCGTCGAGTGGCCGTCATGTCTATGCAGTGCGGCCTATGT
CTCATTAGCGAGTCGTATGTATCATGTCGGGCTCGAATGTTGCACACGTCTGC
GTAATGGTGACCGCTAGTCCCASATGGTGCTTCGTAGCCACAAATGTCGTTAG
GTAGACCGACGTTATCGCGCTATACCCGATGTCAACGCGAGTTAGACCGTAT
CGTCCCCAGTGCCCTAAGATGGTCAAGCGTGCTCCTACGTTAGTATCAGTTTC
CCTATTGGTACGTCTGGCGTACTTCTGAAACGTGATGGGCGGCTGGTTACCCG
TATATGGGCTCGGTTGACCTCTATTGGGCGTTGTTGACCCGAATTCGGTATCC
TCGTCGTTAAATGGCGAACGTCGTCTGCTATAGGCAAACGTCTGTCGGTCATG
GCAAATGTTACTCGTGTGTGCAAGAAATTACTCGCTGTCgtcgacccgggaattccggaa
aaaaaaaaaaaaaaaaaaactgcaggcgtaccagctttccctatagtgagtcgtatta.

18. A DNA molecule according to claim 1 comprising the sequence, wherein capitalized bases refer to Tag gene sequence:
gcatgcaattaaccctcactaaagggacgcgtacgtaagcttGATAAGCGTTCACAGCTCGGCAATAC
CTGTGACGAGCTGCTCGCAAGATTTACGCAGTGTGGCTATACTTGACAGTGAT
GGCGCTTACTTCAGATGTATGGGTGATACTTCGCTATATGGGTGGTCACTTCT
CTATGGCGCGTGACAATGTACTATGGAGCGGTCAATGTCAGTACGGATCGCG
TCGATCTAGGTGACTACGCACGCCTCTGGAGTAAATCGAGTGCTCCGTGCGA
AATACGCGGTCATCGTGCGAATAACCGAGTCATCGTGAGTAGTATGAACGTG
TCGTGTTATGCAGCGGTATGTCGTGCTATAATGGCGTCTGTCGTGCTCATAAG
GTTCCTCTGATGTGCTAGACGTGTCCATCGAGCTGCATAGCTATACTTCGAGT
CACTTGGGATACTTCGATAGCGTTGTGAATAGTGTCGTAGGCTCTCGGGCACG
TTGTTAAACTGTTGCCGCCAATTCAAGATTAGTCCAGCTCGTACTATCGAATA
CACCATCGTCGTATCGAATAATCGCACCTCGTAGGAGTCAGTTGCCACTCGTT
GATAGTCAACCAAGCTCGTTAGATAGTAGCCCAGATCCTACGAGATGAGCTA
CGTAACTACAGTGATAGCATATAGGGTACGCTAGAATGCCAGGTCGTAGTCG
AATTAGTCAGGTTGGATGTCTACTAGTTGACTTGGAGTATGCCATGAAGACTC
GTCCCTCGATATCAATACTCGTCCGCAGGTGAACACTGTAGTCGGTGCTAGTG
CCCACTTCTCGGTATGTGTCCTCAATTATCGAGTAGGATTCTAATCAATCGTC
GCGGCTCACTAATTGTCTGCGGTGGCTACTAATGGTTACGGTGCCTGACTAAT
CGTGTAGGTGTCTAATACATCGTGATACGGGCGATATAATGCTCGATACGGC
AAATATAGCTCCGTCCGGTGGATCCAGATCGCAGGGTATCGCATCGACAGAC
CTGGTATCGTCGTGACGAACGTGCTACTCGCTTATCGGGCCTGCTACATCAGT
GGCGATGTTCGTAACCCTTAGCCGATCTTCTTACTTACGAGGCTACTATTCGA
TCAAACTCGCCTATCTGGTAATAACTGCGGTGATCTGGTAGCCACTACGTGCG
CCTGGTAGCAAATACGGCGAGCTGGTATCACTATCGGCTCAGTGGTCCGACA
TAGTGCCCAGTGGTTCGCATAACTGCCGCTGGGTCCAATATAACACGCAGTC
GTCAATCATACGAGCCGATGGTCAGCAATAGCGCCTGTGGTGACACTATGCC
ACCTCTGGTCTAATATAGCGCCCTGTGGTCGTATAATCGAGCGCGTAATCGTA
TATCCGACTGTAGGTGCGTAACTCGCGACTAGGTGGCTCTAATCTGCGTTGGT
TGTCGCTCACAGTGTCTGGTGTTCGATACCCGGATCGGGTTCCGTAATCTTGG
CATCGAGGTTTCGTACATGTCACGCGGTCTCGTTCATTCTCGGTGGTGCTCAG
TACATCCAGTGGTGAGTCGCTACATCACACGGTGATCCGGCTAAACCTCTGG
GCATCCGTATTAAGCGACATTCCTACGACTTATCAGCACGTCCTACGGTATAA
CAAGGCGTGCTACGGTCTAACGACGCTGGTAGCAGTCTATCAGATCGCTAGT
ACGAGTTAGAGATGCTTAGTACGCCTTCGAATCTATGATGCTCGTGCTCACGC
GATGCACTCGGATTATGGCACATGCACTCGCGTAATGACGCTGCATCGCTCA
GTATGATCCATGAGCGCCGTGAATGACGCATGAGCCTCGTATCGAGTGCATG
AGCTGTCTTTCACATGATACATCGCTCTAAATCATCATGCGACAGTCTCGACA
GCAGCTCAGCATCTATGCATCATGTGCCTCACTAGGACATCATGCTCGACTCT
GAGACACTGATCGAGCATTAAGACtctagagcggccgccgactagtgagctcgtcgaccccgggaatt
ccggaaaaaaaaaaaaaaaaaaaaactgcaggcgtaccagctttccctatagtgagtcgtatta.

19. A DNA molecule according to claim 1 comprising the sequence, wherein capitalized bases refer to Tag gene
sequence:gcatgcaattaaccctcactaaagggacgcgtacgtaagcttGATAAGCGTTCACAGCTCGGC
AATACCTGTGACGAGCTGCTCGCAAGATTTACGCAGTGTGGCTATACTTGAC
AGTGATGGCGCTTACTTCAGATGTATGGGTGATACTTCGCTATATGGGTGGTC
ACTTCTCTATGGCGCGTGACAATGTACTATGGAGCGGTCAATGTCAGTACGG
ATCGCGTCGATCTAGGTGACTACGCACGCCTCTGGAGTAAATCGAGTGCTCC
GTGCGAAATACGCGGTCATCGTGCGAATAACCGAGTCATCGTGAGTAGTATG
AACGTGTCGTGTTATGCAGCGGTATGTCGTGCTATAATGGCGTCTGTCGTGCT
CATAAGGTTCCTCTGATGTGCTAGACGTGTCCATCGAGCTGCATAGCTATACT
TCGAGTCACTTGGGATACTTCGATAGCGTTGTGAATAGTGTCGTAGGCTCTCG
GGCACGTTGTTAAACTGTTGCCGCCAATTCAAGATTAGTCCAGCTCGTACTAT
CGAATACACCATCGTCGTATCGAATAATCGCACCTCGTAGGAGTCAGTTGCC
ACTCGTTGATAGTCAACCAAGCTCGTTAGATAGTAGCCCAGATCCTACGAGA
TGAGCTACGTAACTACAGTGATAGCATATAGGGTACGCTAGAATGCCAGGTC
GTAGTCGAATTAGTCAGGTTGGATGTCTACTAGTTGACTTGGAGTATGCCATG
AAGACTCGTCCCTCGATATCAATACTCGTCCGCAGGTGAACACTGTAGTCGGT
GCTAGTGCCCACTTCTCGGTATGTGTCCTCAATTATCGAGTAGGATTCTAATC
AATCGTCGCGGCTCACTAATTGTCTGCGGTGGCTACTAATGGTTACGGTGCCT
GACTAATCGTGTAGGTGTCTAATACATCGTGATACGGGCGATATAATGCTCG
ATACGGCAAATATAGCTCCGTCCGGTGGATCCAGATCGCAGGGTATCGCATC
GACAGACCTGGTATCGTCGTGACGAACGTGCTACTCGCTTATCGGGCCTGCTA
CATCAGTGGCGATGTTCGTAACCCTTAGCCGATCTTCTTACTTACGAGGCTAC
TATTCGATCAAACTCGCCTATCTGGTAATAACTGCGGTGATCTGGTAGCCACT
ACGTGCGCCTGGTAGCAAATACGGCGAGCTGGTATCACTATCGGCTCAGTGG
TCCGACATAGTGCCCAGTGGTTCGCATAACTGCCGCTGGGTCCAATATAACA
CGCAGTCGTCAATCATACGAGCCGATGGTCAGCAATAGCGCCTGTGGTGACA
CTATGCCACCTCTGGTCTAATATAGCGCCCTGTGGTCGTATAATCGAGCGCGT
AATCGTATATCCGACTGTAGGTGCGTAACTCGCGACTAGGTGGCTCTAATCTG
CGTTGGTTGTCGCTCACAGTGTCTGGTGTTCGATACCCGGATCGGGTTCCGTA
ATCTTGGCATCGAGGTTTCGTACATGTCACGCGGTCTCGTTCATTCTCGGTGG
TGCTCAGTACATCCAGTGGTGAGTCGCTACATCACACGGTGATCCGGCTAAA
CCTCTGGGCATCCGTATTAAGCGACATTCCTACGACTTATCAGCACGTCCTAC
GGTATAACAAGGCGTGCTACGGTCTAACGACGCTGGTAGCAGTCTATCAGAT
CGCTAGTACGAGTTAGAGATGCTTAGTACGCCTTCGAATCTATGATGCTCGTG
CTCACGCGATGCACTCGGATTATGGCACATGCACTCGCGTAATGACGCTGCA
TCGCTCAGTATGATCCATGAGCGCCGTGAATGACGCATGAGCCTCGTATCGA
GTGCATGAGCTGTCTTTCACATGATACATCGCTCTAAATCATCATGCGACAGT
CTCGACAGCAGCTCAGCATCTATGCATCATGTGCCTCACTAGGACATCATGCT
CGACTCTGAGACACTGATCGAGCATTAAGACTCTAGACTCTGTGCCATGATC
GTGAGTTGTCGCAGTGTCTGTACCAATACTCTGGTGGAGCTATATAAGCCGCT
GTTGCGTAAATCAACGGCATGATCCCTATGACCGCGTCATGCTAACTGATAC
ACGCTGCTCGAACAGTGATACGCACACTGATAACTATGCGCAGACGCTTGAA
ACGATGTGACATCGCTTCTAGAGTATGAGCCGCAATGCACGACTGATACTCG
ATATGAGCAGCAGTCGGCTATGATTTGCAATGCTTGCAGTATGTATCCTGATC
GTGCGTGCGATGTCTGATAATACGCTCGCATGATATGTATTGCGCTCAGATGC
TGGAGATATGCCATGCGTGCTGTCAGTATGCCATGTATGCTGATATGTCGCGA
TCTATGTGGTGACTATGAGATCCATGTGATGACGTTGCAGTCTCTGTGACCTT
ATCGACGCGCATGTGAGCCTATAGACAGCGATGTGAGCACTCTCATCTGCGG
ATCAGTCTATCCTCGCTGATGCTCAGTGATACACGCTGATGCACGTAGTGAGC
ATCCTGTGCTCGCATATACCGCTGCTGCACTGATATGAGCCAGTGCTGCTGCT
CTCTACGGAGTGTGCTCGGCTATAACAGCGAGTGCTACGCCTAAACTGGCTG
TCTAGCACTGTAGCTGGTGCATGTACTCGACTGCCGCTGCATCTACTATAAGA
CTCTGACATTAGCGTATAGGCTGATACATTAGCTCGGATGCTATCAGCTTGCG
CCTATTATATGCCTGACGCGGGATCTATCAGAACGACTCGGTAGCTCATATAC
TGGATCACGGTGCCACAACATGCTACACGAGGTCTCAGACTCTATCCCGTGG
ACTCAACGTGCATCTGCTATGCTGAGCGCGTATCTGTGTACCTGTCCGATGCT
CTGATCTACACTGCCGTGATCGTTATATGACGAGACTGTGCGCTCATAGCCGA
CACTGTGCTCGATAAGACCACGCTGTGCGGATATAGTCGACCTAGTGCATCCT
CGTGGCATCATGCGTCTCCTCAGTAGGTCTGCGACTGATCCTAGTGCAATGCG
TCTGAGCCTGAGCTACAGCGATATAGCCTGGATTGTGAGCGTATTTGCTGTCA
GAACCTCAGCTCATCATGTATGATGCTGTACCATCCTGCGATACTGAAGATGC
ACCGCTATAATGCGAGCCTCTCCGCTAAAGTGGAAGCTGCTCGTTCTCAATGC
GAGCGAGTCGAATCCAATGCCGTAGCTGCGATAACGATGCCGCTGACTCTAC
GGTAATGCACGATCCTCTACATTGATAGCAGATAGTCTAACGGGATAGCATA
GGTGCAAGGCTCCTAGCATGTAGTCACAGGTGCTCAGATATAGTCATCGCTG
CAATCAGCTAGTCATCTTGTCAGGATGCTACTCACTGCGTGCAGAAGATTCGC
ACGACTTCAGAGGATGGCACTCGTCATTAGAGTGATGTTCTCGGATCGACAC
TGCTGGTCTGCGAATGACTCGCATTCACTAACATGGAGCATCGTTATCTAAAG
GGGATGCACGTTATCGTCGAGTGGCCGTCATGTCTATGCAGTGCGGCCTATGT
CTCATTAGCGAGTCGTATGTATCATGTCGGGCTCGAATGTTGCACACGTCTGC
GTAATGGTGACCGCTAGTCCCACATGGTGCTTCGTAGCCACAAATGTCGTTAG
GTAGACCGACGTTATCGCGCTATACCCGATGTCAACGCGAGTTAGACCGTAT
CGTCCCCAGTGCCCTAAGATGGTCAAGCGTGCTCCTACGTTAGTATCAGTTTC
CCTATTGGTACGTCTGGCGTACTTCTGAAACGTGATGGGCGGCTGGTTACCCG
TATATGGGCTCGGTTGACCTCTATTGGGCGTTGTTGACCCgaattccggaaaaaaaaaaaa
aaaaaaaaactgcaggcgtaccagctttccctatagtgagtcgtatta.

20. A DNA molecule according to claim 1 further comprising at least two additional restriction sites.

21. A DNA molecule according to claim 20 comprising the sequence wherein capitalized bases refer to Tag gene sequence
gcatgcaattaaccctcactaaagggacgcgtacgtaagcttGATAAGCGTTCACAGCTCGGCAATAC
CTGTGACGAGCTGCTCGCAAGATTTACGCAGTGTGGCTATACTTGACAGTGAT
GGCGCTTACTTCAGATGTATGGGTGATACTTCGCTATATGGGTGGTCACTTCT
CTATGGCGCGTGACAATGTACTATGGAGCGGTCAATGTCAGTACGGATCGCG
TCGATCTAGGTGACTACGCACGCCTCTGGAGTAAATCGAGTGCTCCGTGCGA
AATACGCGGTCATCGTGCGAATAACCGAGTCATCGTGAGTAGTATGAACGTG
TCGTGTTATGCAGCGGTATGTCGTGCTATAATGGCGTCTGTCGTGCTCATAAG
GTTCCTCTGATGTGCTAGACGTGTCCATCGAGCTGCATAGCTATACTTCGAGT
CACTTGGGATACTTCGATAGCGTTGTGAATAGTGTCGTAGGCTCTCGGGCACG
TTGTTAAACTGTTGCCGCCAATTCAAGATTAGTCCAGCTCGTACTATCGAATA
CACCATCGTCGTATCGAATAATCGCACCTCGTAGGAGTCAGTTGCCACTCGTT
GATAGTCAACCAAGCTCGTTAGATAGTAGCCCAGATCCTACGAGATGAGCTA
CGTAACTACAGTGATAGCATATAGGGTACGCTAGAATGCCAGGTCGTAGTCG
AATTAGTCAGGTTGGATGTCTACTAGTTGACTTGGAGTATGCCATGAAGACTC
GTCCCTCGATATCAATACTCGTCCGCAGGTGAACACTGTAGTCGGTGCTAGTG
CCCACTTCTCGGTATGTGTCCTCAATTATCGAGTAGGATTCTAATCAATCGTC
GCGGCTCACTAAITGTCTGCGGTGGCTACTAATGGTTACGGTGCCTGACTAAT
CGTGTAGGTGTCTAATACATCGTGATACGGGCGATATAATGCTCGATACGGC
AAATATAGCTCCGTCCGGTGGATCCAGATCGCAGGGTATCGCATCGACAGAC
CTGGTATCGTCGTGACGAACGTGCTACTCGCTTATCGGGCCTGCTACATCAGT
GGCGATGTTCGTAACCCTTAGCCGATCTTCTTACTTACGAGGCTACTATTCGA
TCAAACTCGCCTATCTGGTAATAACTGCGGTGATCTGGTAGCCACTACGTGCG
CCTGGTAGCAAATACGGCGAGCTGGTATCACTATCGGCTCAGTGGTCCGACA
TAGTGCCCAGTGGTTCGCATAACTGCCGCTGGGTCCAATATAACACGCAGTC
GTCAATCATACGAGCCGATGGTCAGCAATAGCGCCTGTGGTGACACTATGCC
ACCTCTGGTCTAATATAGCGCCCTGTGGTCGTATAATCGAGCGCGTAATCGTA
TATCCGACTGTAGGTGCGTAACTCGCGACTAGGTGGCTCTAATCTGCGTTGGT
TGTCGCTCACAGTGTCTGGTGTTCGATACCCGGATCGGGTTCCGTAATCTTGG
CATCGAGGTTTCGTACATGTCACGCGGTCTCGTTCATTCTCGGTGGTGCTCAG
TACATCCAGTGGTGAGTCGCTACATCACACGGTGATCCGGCTAAACCTCTGG
GCATCCGTATTAAGCGACATTCCTACGACTTATCAGCACGTCCTACGGTATAA
CAAGGCGTGCTACGGTCTAACGACGCTGGTAGCAGTCTATCAGATCGCTAGT
ACGAGTTAGAGATGCTTAGTACGCCTTCGAATCTATGATGCTCGTGCTCACGC
GATGCACTCGGATTATGGCACATGCACTCGCGTAATGACGCTGCATCGCTCA
GTATGATCCATGAGCGCCGTGAATGACGCATGAGCCTCGTATCGAGTGCATG
AGCTGTCTTTCACATGATACATCGCTCTAAATCATCATGCGACAGTCTCGACA
GCAGCTCAGCATCTATGCATCATGTGCCTCACTAGGACATCATGCTCGACTCT
GAGACACTGATCGAGCATTAAGACTCTAGACTCTGTGCCATGATCGTGAGTT
GTCGCAGTGTCTGTACCAATACTCTGGTGGAGCTATATAAGCCGCTGTTGCGT
AAATCAACGGCATGATCCCTATGACCGCGTCATGCTAACTGATACACGCTGC
TCGAACAGTGATACGCACACTGATAACTATGCGCAGACGCTTGAAACGATGT
GACATCGCTTCTAGAGTATGAGCCGCAATGCACGACTGATACTCGATATGAG
CAGCAGTCGGCTATGATTTGCAATGCTTGCAGTATGTATCCTGATCGTGCGTG
CGATGTCTGATAATACGCTCGCATGATATGTATTGCGCTCAGATGCTGGAGAT
ATGCCATGCGTGCTGTCAGTATGCCATGTATGCTGATATGTCGCGATCTATGT
GGTGACTATGAGATCCATGTGATGACGTTGCAGTCTCTGTGACCTTATCGACG
CGCATGTGAGCCTATAGACAGCGATGTGAGCACTCTCATCTGCGGATCAGTC
TATCCTCGCTGATGCTCAGTGATACACGCTGATGCACGTAGTGAGCATCCTGT
GCTCGCATATACCGCTGCTGCACTGATATGAGCCAGTGCTGCTGCTCTCTACG
GAGTGTGCTCGGCTATAACAGCGAGTGCTACGCCTAAACTGGCTGTCTAGAA
CTGTAGCTGGTGCATGTACTCGACTGCCGCTGCATCTACTATAAGACTCTGAC
ATTAGCGTATAGGCTGATACATTAGCTCGGATGCTATCAGCTTGCGCCTATTA
TATGCCTGACGCGGGATCTATCAGAACGACTCGGTAGCTCATATACTGGATC
ACGGTGCCACAACATGCTACACGAGGTCTCAGACTCTATCCCGTGGACTCAA
CGTGCATCTGCTATGCTGAGCGCGTATCTGTGTACCTGTCCGATGCTCTGATC
TACACTGCCGTGATCGTTATATGACGAGACTGTGCGCTCATAGCCGACACTGT
GCTCGATAAGACCACGCTGTGCGGATATAGTCGACCTAGTGCATCCTCGTGG
CATCATGCGTCTCCTCAGTAGGTCTGCGACTGATCCTAGTGCAATGCGTCTGA
GCCTGAGCTACAGCGATATAGCCTGGATTGTGAGCGTATTTGCTGTCAGAAC
CTCAGCTCATCATGTATGATGCTGTACCATCCTGCGATACTGAAGATGCACCG
CTATAATGCGAGGCTCTCCGCTAAAGTGGAAGCTGCTCGTTCTCAATGCGAG
CGAGTCGAATTCAATGCCGTAGCTGCGATAACGATGCCGCTGACTCTACGGT
AATGCACGATCCTCTACATTGATAGCAGATAGTCTAACGGGATAGCATAGGT
GCAAGGCTCCTAGCATGTAGTCACAGGTGCTCAGATATAGTCATCGCTGCAA
TCAGCTAGTCATCTTGTCAGGATGCTACTCACTGCGTGCAGAAGATTCGCACG
ACTTCAGAGGATGGCACTCGTCATTAGAGTGATGTTCTCGGATCGACACTGCT
GGTCTGCGAATGACTCGCATTCACTAACATGGAGCATCGTTATCTAAAGGGG
ATGCACGTTATCGTCGAGTGGCCGTCATGTCTATGCAGTGCGGCCTATGTCTC
ATTAGCGAGTCGTATGTATCATGTCGGGCTCGAATGTTGCACACGTCTGCGTA
ATGGTGACCGCTAGTCCCACATGGTGCTTCGTAGCCACAAATGTCGTTAGGTA
GACCGACGTTATCGCGCTATACCCGATGTCAACGCGAGTTAGACCGTATCGT
CCCCAGTGCCCTAAGATGGTCAAGCGTGCTCCTACGTTAGTATCAGTTTCCCT
ATTGGTACGTCTGGCGTACTTCTGAAACGTGATGGGCGGCTGGTTACCCGTAT
ATGGGCTCGGTTGACCTCTATTGGGCGTTGTTGACCCgaattccggaaaaaaaaaaaaaaaa
aaaaactgcaggcgtaccagctttccctatagtgagtcgtatta.

22. A method of providing a control for an assay, said assay comprising providing labeled nucleic acid and hybridizing said Labeled nucleic acid to a nucleic acid array, said method comprising spiking said labeled nucleic acid with labeled Tag gene nucleic acid, wherein said nucleic acid array has probes complementary to said Tag gene.

23. A method according to claim 22 wherein said nucleic acid is RNA.

24. A method according to claim 22 wherein said nucleic acid is DNA.

25. A method according to claim 22 wherein said Tag gene is selected from the group consisting of Tags A, B, C, D, E, F, G, H, I, J, N, O, Q, Tag IN, Tag IQ and Tag IQ.EX

26. A method of analyzing the expression of one or more genes, said method comprising:
(a) providing a pool of target nucleic acids comprising RNA transcripts of one or more of said genes, or nucleic acids derived therefrom using said RNA transcripts as templates;
(b) providing a spike sample comprising RNA transcribed from a Tag gene or Tag nucleic acids derived from said Tag gene RNA using said Tag gene RNA as template;
(c) hybridizing said pool of target nucleic acids and said spike sample to an array of oligonucleotide probes immobilized on a surface, said array comprising more than 100 different oligonucleotides, at least some of which comprise control probes and at least some of which comprise probes complementary to said Tag gene or said nucleic acid derived from said Tag gene RNA, wherein each different oligonucleotide is localized in a predetermined region of said surface, the density of said different oligonucleotides is greater than about 60 different oligonucleotides per 1 cm², and at least some of said oligonucleotide probes are complementary to said RNA transcripts or said nucleic acids derived therefrom using said RNA transcripts;
(d) quantifying the hybridization of said nucleic acids to said array, wherein said quantification is proportional to the expression level of said genes; and
(e) quantifying the hybrization of said spike sample to said array.

27. A method according to claim 26 wherein said Tag gene is selected from the group consisting of Tags A, B, C, D, E, F, G, H, I, J, N, O, Q, Tag IN, Tag IQ and Tag IQ.EX

28. A DNA molecule comprising a Tag gene, said Tag gene comprising at least 5 Tag sequences or their complement.

29. A DNA molecule according to claim 28 wherein said Tag sequences are selected from Seq. Id. Nos. 1-2050.

30. A DNA molecule according to claim 29 wherein said Tag gene sequences are selected from the group consisting of Tags A, B, C, D, E, F, G, H, I, J, N, O, Q, Tag IN, Tag IQ and Tag IQ.EX
